SEQUENCE LISTING (121 sequences)

SEQ ID NO: 1 - protein, 118 aa, Artificial Sequence
  Heavy chain variable region
EVQLVQSGAEVKKPGSSVKVSCKASGGTFRSFGISWVRQAPGQGLEWMGGFIPVLGTANYAQKFQGRVTIIADKSTNTAYMELSSLRSEDTAVYYCARRGNWNPFDPWGQGTLVTVSS

SEQ ID NO: 2 - protein, 5 aa, Artificial Sequence
  HCDR1 according to Kabat
SFGIS

SEQ ID NO: 3 - protein, 17 aa, Artificial Sequence
  HCDR2 according to Kabat
GFIPVLGTANYAQKFQG

SEQ ID NO: 4 - protein, 9 aa, Artificial Sequence
  HCDR3 according to Kabat
RGNWNPFDP

SEQ ID NO: 5 - protein, 120 aa, Artificial Sequence
  Heavy chain variable region
QVQLVQSGAEVKKPGSSVKVSCKASGDAFKSKTFTISWVRQAPGQGLEWLGGIIPLFGTITYAQKFQGRVTITADKSTNTAFMELSSLRSEDTAMYYCTRRGNWNPFDPWGQGTLVTVSS

SEQ ID NO: 6 - protein, 7 aa, Artificial Sequence
  HCDR1 according to Kabat
SKTFTIS

SEQ ID NO: 7 - protein, 17 aa, Artificial Sequence
  HCDR2 according to Kabat
GIIPLFGTITYAQKFQG

SEQ ID NO: 8 - protein, 120 aa, Artificial Sequence
  Heavy chain variable region
EVQLVQSGSELKKPGSSVKVSCKASGVTFNSRTFTISWVRQAPGQGLEWLGSIIPIFGTITYAQKFQGRVTITADKSTSTAFMELTSLRSEDTAIYYCTRRGNWNPFDPWGQGTLVTVSS

SEQ ID NO: 9 - protein, 7 aa, Artificial Sequence
  HCDR1 according to Kabat
SRTFTIS

SEQ ID NO: 10 - protein, 17 aa, Artificial Sequence
  HCDR2 according to Kabat
SIIPIFGTITYAQKFQG

SEQ ID NO: 11 - protein, 123 aa, Artificial Sequence
  Heavy chain variable region
QVQLVQSGGGLVQPGGSLRLSCATSGFKFSSYALSWVRQAPGKGLEWVSGISGSGRTTWYADSVKGRFTISRDNSKNTLYLQMNSLRAEDTAVYYCARDGGYSYGPYWYFDLWGRGTLVTVSS

SEQ ID NO: 12 - protein, 5 aa, Artificial Sequence
  HCDR1 according to Kabat
SYALS

SEQ ID NO: 13 - protein, 17 aa, Artificial Sequence
  HCDR2 according to Kabat
GISGSGRTTWYADSVKG

SEQ ID NO: 14 - protein, 14 aa, Artificial Sequence
  HCDR3 according to Kabat
DGGYSYGPYWYFDL

SEQ ID NO: 15 - protein, 126 aa, Artificial Sequence
  Heavy chain variable region
EVQLVQSGAEVKKPGESLKISCKGSGYSFTRFWIGWVRQMPGKGLEWMGIIYPGDSDTRYSPSFQGQVTISADKSTSTAYLQWSSLKASDTGMYYCVRHIRYFDWSEDYHYYLDVWGKGTTVTVSS

SEQ ID NO: 16 - protein, 5 aa, Artificial Sequence
  HCDR1 according to Kabat
RFWIG

SEQ ID NO: 17 - protein, 17 aa, Artificial Sequence
  HCDR2 according to Kabat
IIYPGDSDTRYSPSFQG

SEQ ID NO: 18 - protein, 17 aa, Artificial Sequence
  HCDR3 according to Kabat
HIRYFDWSEDYHYYLDV

SEQ ID NO: 19 - protein, 126 aa, Artificial Sequence
  Heavy chain variable region
EVQLVESGAEVKKPGESLKISCKGSGYSFTRYWIGWVRQMPGKGLEWMGIIYPGDSDTRYSPSFQGQVTISADKSISTAYLQWSSLKASDTAMYYCVRNIRYFVWSEDYHYYMDVWGKGTTVTVSS

SEQ ID NO: 20 - protein, 5 aa, Artificial Sequence
  HCDR1 according to Kabat
RYWIG

SEQ ID NO: 21 - protein, 17 aa, Artificial Sequence
  HCDR3 according to Kabat
NIRYFVWSEDYHYYMDV

SEQ ID NO: 22 - protein, 124 aa, Artificial Sequence
  Heavy chain variable region
EVQLVESGGGLVQPGRSLRLSCATSGFNFDDYTMHWVRQAPGKGLEWVSDISWSSGSIGYADSVKGRFTISRDNAKNSLWLQMNSLRTEDTALYFCAKDHRGYGDYEGGGFDYWGQGTLVTVSS

SEQ ID NO: 23 - protein, 5 aa, Artificial Sequence
  HCDR1 according to Kabat
DYTMH

SEQ ID NO: 24 - protein, 17 aa, Artificial Sequence
  HCDR2 according to Kabat
DISWSSGSIGYADSVKG

SEQ ID NO: 25 - protein, 15 aa, Artificial Sequence
  HCDR3 according to Kabat
DHRGYGDYEGGGFDY

SEQ ID NO: 26 - protein, 121 aa, Artificial Sequence
  Heavy chain variable region
EVQLVQSGAEVKKPGSSVKVSCKASGGIFSTYAISWVRQAPGQGLEWMGGIIPIFDTPNYAQKFQGRVTITADKSTSTAYMDLSSLRSEDTAVYYCAKNVRGYSAYDLDYWGQGTLVTVSS

SEQ ID NO: 27 - protein, 5 aa, Artificial Sequence
  HCDR1 according to Kabat
TYAIS

SEQ ID NO: 28 - protein, 17 aa, Artificial Sequence
  HCDR2 according to Kabat
GIIPIFDTPNYAQKFQG

SEQ ID NO: 29 - protein, 12 aa, Artificial Sequence
  HCDR3 according to Kabat
NVRGYSAYDLDY

SEQ ID NO: 30 - protein, 121 aa, Artificial Sequence
  Heavy chain variable region
QVQLVQSGSELKKPGASVKVSCKASGYTFTSYSMNWVRQAPGQGLEWMGWINTNTGNPTYAQGFTGRFVFSLDTSVSTAYLQISSLKAEDTAVYYCARDHDFRTGRAFDIWGQGTTVTVSS

SEQ ID NO: 31 - protein, 5 aa, Artificial Sequence
  HCDR1 according to Kabat
SYSMN

SEQ ID NO: 32 - protein, 17 aa, Artificial Sequence
  HCDR2 according to Kabat
WINTNTGNPTYAQGFTG

SEQ ID NO: 33 - protein, 12 aa, Artificial Sequence
  HCDR3 according to Kabat
DHDFRTGRAFDI

SEQ ID NO: 34 - protein, 124 aa, Artificial Sequence
  Heavy chain variable region
EVQLVESGGDVVQPGRSLRLSCAASGFTFSSYGMHWVRQAPGKGLEWVAVISYDGSNKYYADSVKGRFTISRDNSKSTLFLQMNSLRAEDTAVYFCVRGLPITMVRGAYSFDYWGQGTLVTVSS

SEQ ID NO: 35 - protein, 5 aa, Artificial Sequence
  HCDR1 according to Kabat
SYGMH

SEQ ID NO: 36 - protein, 17 aa, Artificial Sequence
  HCDR2 according to Kabat
VISYDGSNKYYADSVKG

SEQ ID NO: 37 - protein, 15 aa, Artificial Sequence
  HCDR3 according to Kabat
GLPITMVRGAYSFDY

SEQ ID NO: 38 - protein, 124 aa, Artificial Sequence
  Heavy chain variable region
EVQLVQSGAEVKKPGSSVKVSCKASGDTFNTYSITWVRQAPGQGLEWMGSIVPIFGTINNAQKFQGRVTITADKSANTAYMELSSLRSEDTAVYYCARDNTMVRGVDYYYMDVWGKGTMVTVSS

SEQ ID NO: 39 - protein, 5 aa, Artificial Sequence
  HCDR1 according to Kabat
TYSIT

SEQ ID NO: 40 - protein, 17 aa, Artificial Sequence
  HCDR2 according to Kabat
SIVPIFGTINNAQKFQG

SEQ ID NO: 41 - protein, 15 aa, Artificial Sequence
  HCDR3 according to Kabat
DNTMVRGVDYYYMDV

SEQ ID NO: 42 - protein, 121 aa, Artificial Sequence
  Heavy chain variable region
QVQLVQSGAEVKKPGSSVKVSCKASGDTFRSYGITWVRQAPGQGLEWMGGIIPIFGTTNYAQKFQGRVTITADKSTSTVYMELSSLRSEDTAVYYCARRRGYSNPHWLDPWGQGTLVTVSS

SEQ ID NO: 43 - protein, 5 aa, Artificial Sequence
  HCDR1 according to Kabat
SYGIT

SEQ ID NO: 44 - protein, 17 aa, Artificial Sequence
  HCDR2 according to Kabat
GIIPIFGTTNYAQKFQG

SEQ ID NO: 45 - protein, 12 aa, Artificial Sequence
  HCDR3 according to Kabat
RRGYSNPHWLDP

SEQ ID NO: 46 - protein, 422 aa, Artificial Sequence
  Heavy chain sequence
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGN

SEQ ID NO: 47 - protein, 425 aa, Artificial Sequence
  Heavy chain
QVQLVQSGAEVKKPGSSVRVSCKASGGTFNTYAINWVRQAPGQGLEWVGRIIPIFGTANYAQKFQGRVTISADKSTTTAYMELSSLRSEDTAVFYCAKDETGYSSSNFQHWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGN

SEQ ID NO: 48 - protein, 5 aa, Artificial Sequence
  HCDR1 according to Kabat
TYAIN

SEQ ID NO: 49 - protein, 17 aa, Artificial Sequence
  HCDR2 according to Kabat
RIIPIFGTANYAQKFQG

SEQ ID NO: 50 - protein, 12 aa, Artificial Sequence
  HCDR3 according to Kabat
DETGYSSSNFQH

SEQ ID NO: 51 - protein, 451 aa, Artificial Sequence
  Heavy chain sequence
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 122 aa, Artificial Sequence
  Heavy chain variable region
QVQLVQSGAEVKKPGASVKVSCKASGYTFTSYGISWVRQAPGQGLEWMGWISAYNANTNYAQKLQGRVTMTTDTSTSTAYMELRSLRSDDTAVYYCAKDRHWHWWLDAFDYWGQGTLVTVSS

SEQ ID NO: 53 - protein, 5 aa, Artificial Sequence
  HCDR1 according to Kabat
SYGIS

SEQ ID NO: 54 - protein, 17 aa, Artificial Sequence
  HCDR2 according to Kabat
WISAYNANTNYAQKLQG

SEQ ID NO: 55 - protein, 13 aa, Artificial Sequence
  HCDR3 according to Kabat
DRHWHWWLDAFDY

SEQ ID NO: 56 - protein, 122 aa, Artificial Sequence
  Heavy chain variable region
QVQLVQSGAEVKKPGASVKVSCKASGYTFTSYGISWVRQAPGQGLEWMGWISAYNANTNYAQKLQGRVTMTTDTSTSTAYMELRSLRSDDTAVYYCAKDLYGHWWLDAFDYWGQGTLVTVSS

SEQ ID NO: 57 - protein, 13 aa, Artificial Sequence
  HCDR3 according to Kabat
DLYGHWWLDAFDY

SEQ ID NO: 58 - protein, 122 aa, Artificial Sequence
  Heavy chain variable region
QVQLVQSGAEVKKPGASVKVSCKASGYTFTSYGISWVRQAPGQGLEWMGWISAYNANTNYAQKLQGRVTMTTDTSTSTAYMELRSLRSDDTAVYYCAKGPGSHWWLDAFDYWGQGTLVTVSS

SEQ ID NO: 59 - protein, 13 aa, Artificial Sequence
  HCDR3 according to Kabat
GPGSHWWLDAFDY

SEQ ID NO: 60 - protein, 122 aa, Artificial Sequence
  Heavy chain variable region
QVQLVQSGAEVKKPGASVKVSCKASGYTFTSYGISWVRQAPGQGLEWMGWISAYNANTNYAQKLQGRVTMTTDTSTSTAYMELRSLRSDDTAVYYCAKDRGWHWWLDAFDYWGQGTLVTVSS

SEQ ID NO: 61 - protein, 13 aa, Artificial Sequence
  HCDR3 according to Kabat
DRGWHWWLDAFDY

SEQ ID NO: 62 - protein, 122 aa, Artificial Sequence
  Heavy chain variable region
QVQLVQSGAEVKKPGASVKVSCKASGYTFTSYGISWVRQAPGQGLEWMGWISAYNANTNYAQKLQGRVTMTTDTSTSTAYMELRSLRSDDTAVYYCAKDRHWHWWLDGFDYWGQGTLVTVSS

SEQ ID NO: 63 - protein, 13 aa, Artificial Sequence
  HCDR3 according to Kabat
DRHWHWWLDGFDY

SEQ ID NO: 64 - protein, 118 aa, Artificial Sequence
  Heavy chain variable region
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAKGTTGDWFDYWGQGTLVTVSS

SEQ ID NO: 65 - protein, 5 aa, Artificial Sequence
  HCDR1 according to Kabat
SYYMH

SEQ ID NO: 66 - protein, 17 aa, Artificial Sequence
  HCDR2 according to Kabat
IINPSGGSTSYAQKFQG

SEQ ID NO: 67 - protein, 9 aa, Artificial Sequence
  HCDR3 according to Kabat
GTTGDWFDY

SEQ ID NO: 68 - protein, 127 aa, Artificial Sequence
  Heavy chain variable region
EVQLVETGAEVKKPGASVKVSCKASDYIFTKYDINWVRQAPGQGLEWMGWMSANTGNTGYAQKFQGRVTMTRDTSINTAYMELSSLTSGDTAVYFCARSSLFKTETAPYYHFALDVWGQGTTVTVSS

SEQ ID NO: 69 - protein, 7 aa, Artificial Sequence
  Linker 1
ESKYGPP

SEQ ID NO: 70 - protein, 10 aa, Artificial Sequence
  Linker 2
EPKSCDKTHT

SEQ ID NO: 71 - protein, 10 aa, Artificial Sequence
  Linker 3
GGGGSGGGGS

SEQ ID NO: 72 - protein, 12 aa, Artificial Sequence
  Linker 4
ERKSSVESPPSP

SEQ ID NO: 73 - protein, 12 aa, Artificial Sequence
  Linker 5
ERKCSVESPPSP

SEQ ID NO: 74 - protein, 12 aa, Artificial Sequence
  Linker 6
ELKTPLGDTTHT

SEQ ID NO: 75 - protein, 12 aa, Artificial Sequence
  Linker 7
ESKYGPPSPSSP

SEQ ID NO: 76 - protein, 13 aa, Artificial Sequence
  Linker 8
ERKSSVEAPPVAG

SEQ ID NO: 77 - protein, 13 aa, Artificial Sequence
  Linker 9
ERKCSVEAPPVAG

SEQ ID NO: 78 - protein, 14 aa, Artificial Sequence
  Linker 10
ESKYGPPAPEFLGG

SEQ ID NO: 79 - protein, 15 aa, Artificial Sequence
  Linker 11
EPKSCDKTHTSPPSP

SEQ ID NO: 80 - protein, 16 aa, Artificial Sequence
  Linker 12
EPKSCDGGGGSGGGGS

SEQ ID NO: 81 - protein, 16 aa, Artificial Sequence
  Linker 13
GGGGSGGGGSAPPVAG

SEQ ID NO: 82 - protein, 17 aa, Artificial Sequence
  Linker 14
EPKSCDKTHTAPELLGG

SEQ ID NO: 83 - protein, 18 aa, Artificial Sequence
  Linker 15
ERKSSVESPPSPAPPVAG

SEQ ID NO: 84 - protein, 18 aa, Artificial Sequence
  Linker 16
ERKCSVESPPSPAPPVAG

SEQ ID NO: 85 - protein, 19 aa, Artificial Sequence
  Linker 17
ELKTPLGDTTHTAPEFLGG

SEQ ID NO: 86 - protein, 19 aa, Artificial Sequence
  Linker 18
ESKYGPPSPSSPAPEFLGG

SEQ ID NO: 87 - protein, 22 aa, Artificial Sequence
  Linker 19
EPKSCDKTHTSPPSPAPELLGG

SEQ ID NO: 88 - protein, 23 aa, Artificial Sequence
  Linker 20
ERKSSVEEAAAKEAAAKAPPVAG

SEQ ID NO: 89 - protein, 23 aa, Artificial Sequence
  Linker 21
ERKCSVEEAAAKEAAAKAPPVAG

SEQ ID NO: 90 - protein, 24 aa, Artificial Sequence
  Linker 22
ESKYGPPEAAAKEAAAKAPEFLGG

SEQ ID NO: 91 - protein, 27 aa, Artificial Sequence
  Linker 23
EPKSCDKTHTEAAAKEAAAKAPELLGG

SEQ ID NO: 92 - protein, 29 aa, Artificial Sequence
  Linker 24
ELKTPLGDTTHTEAAAKEAAAKAPEFLGG

SEQ ID NO: 93 - protein, 107 aa, Artificial Sequence
  Light chain variable region
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 94 - protein, 6 aa, Artificial Sequence
  LCDR1 according to IMGT
QSISSY

SEQ ID NO: 95 - skipped

SEQ ID NO: 96 - protein, 9 aa, Artificial Sequence
  LCDR3 according to IMGT
QQSYSTPPT

SEQ ID NO: 97 - protein, 107 aa, Artificial Sequence [sequence omitted]

SEQ ID NO: 98 - protein, 214 aa, Artificial Sequence
  Light chain sequence
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 108 aa, Artificial Sequence
  IGKV1-39/jk5 Light chain variable region
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 100 - protein, 98 aa, Artificial Sequence
  CH1 sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 101 - protein, 15 aa, Artificial Sequence
  Hinge
EPKSCDKTHTCPPCP

SEQ ID NO: 102 - protein, 110 aa, Artificial Sequence
  CH2 sequence
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 103 - protein, 107 aa, Artificial Sequence
  Modified CH3 sequence
GQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 107 aa, Artificial Sequence
  Modified CH3 sequence
GQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 214 aa, Artificial Sequence
  Light chain sequence
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 215 aa, Artificial Sequence
  Light chain sequence
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 95 aa, Artificial Sequence
  IgVk1-39 V-region
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 108 - protein, 96 aa, Artificial Sequence
  IgVk 3-20 V-region
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 109 - protein, 95 aa, Artificial Sequence
  IgVk3-15 V-region
EIVMTQSPATLSVSPGERATLSCRASQSVSSNLAWYQQKPGQAPRLLIYGASTRATGIPARFSGSGSGTEFTLTISSLQSEDFAVYYCQQYNNWP

SEQ ID NO: 110 - protein, 96 aa, Artificial Sequence
  IgVL3-21 V-region
SYVLTQPPSVSVAPGETARITCGGDNIGRKSVYWYQQKSGQAPVLVIYYDSDRPSGIPERFSGSNSGNTATLTISRVEAGDEADYYCQVWDGSSDH

SEQ ID NO: 111 - DNA, 321 bp, Artificial Sequence
  common light chain variable region
gacatccagatgacccagtctccatcctccctgtctgcatctgtaggagacagagtcaccatcacttgccgggcaagtcagagcattagcagctacttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccaacgttcggccaagggaccaaggtggagatcaaa

SEQ ID NO: 112 - DNA, 324 bp, Artificial Sequence
  common light chain constant region
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 113 - protein, 11 aa, Artificial Sequence
  CDR1 according to Kabat
RASQSISSYLN

SEQ ID NO: 114 - protein, 7 aa, Artificial Sequence
  CDR2 according to Kabat
AASSLQS

SEQ ID NO: 115 - protein, 9 aa, Artificial Sequence
  CDR3 according to Kabat
QQSYSTPPT

SEQ ID NO: 116 - DNA, 294 bp, Artificial Sequence
  CH1 region
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagagagtt

SEQ ID NO: 117 - DNA, 45 bp, Artificial Sequence
  hinge region
gagcccaaatcttgtgacaaaactcacacatgcccaccgtgccca

SEQ ID NO: 118 - DNA, 330 bp, Artificial Sequence
  CH2 region
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaa

SEQ ID NO: 119 - DNA, 321 bp, Artificial Sequence
  CH3 domain comprising variations L351K and T366K (KK)
gggcagccccgagaaccacaggtgtacaccaagcccccatcccgggaggagatgaccaagaaccaggtcagcctgaagtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttga

SEQ ID NO: 120 - DNA, 321 bp, Artificial Sequence
  CH3 domain comprising variations L351D and L368E (DE)
  CDS (1)..(321)
gggcagccccgagaaccacaggtgtacaccgaccccccatcccgggaggagatgaccaagaaccaggtcagcctgacctgcgaggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttga

SEQ ID NO: 121 - protein, 106 aa, Artificial Sequence
  Synthetic Construct
GQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2023/0210988 A1
(43) Pub. Date: Jul. 6, 2023

(54) MEANS AND METHOD FOR MODULATING IMMUNE CELL ENGAGING EFFECTS

(71) Applicant: Merus N.V., Utrecht (NL)

(72) Inventors: Pieter Fokko VAN LOO, Utrecht (NL); Mark THROSBY, Utrecht (NL)

(21) Appl. No.: 17/759,728

(22) PCT Filed: Jan. 28, 2021
(86) PCT No.: PCT/NL2021/050051

§ 371 Date: Jul. 29, 2022

(30) Foreign Application Priority Data
Jan. 29, 2020 (NL) 2024786

(51) Int. Cl. A61K 39/395; A61P 35/00

(52) U.S. Cl. CPC A61K 39/39558; A61K 39/3955; A61P 35/00

(57) ABSTRACT

The invention relates to a composition comprising a multivalent antibody comprising a first variable domain that binds a first tumor antigen (TA1), a second variable domain that binds a second tumor antigen (TA2) and a third variable domain that binds an immune cell engaging antigen (IEA); and wherein the composition further comprises a second binding molecule that binds TA1 or TA2. The invention also relates to a kit of parts comprising the multivalent antibody and second binding molecule, and to means and methods for the treatment of cancer comprising administering to the subject in need thereof the multivalent antibody and second binding molecule.

### BACKGROUND OF THE INVENTION

The invention relates to means and methods of activating immune cells in a subject and to a method of treating cancer in a subject with immune cell engaging binding molecules. In one aspect the invention relates to a composition comprising two or more binding molecules wherein a first is a multivalent antibody with a variable domain that binds an immune cell activating molecule and two variable domains that bind two tumor antigens (TA1 and TA2). A second of the binding molecules is a binding molecule that binds TA1 or TA2. The invention also relates to a kit of parts comprising said antibodies, and to a method for treating cancer with said binding molecules.

Cancer remains one of the leading causes of death. Advances in treatments on various fronts have resulted in improved treatments and survival in certain indications and patient populations. A promising direction is the development of tumor targeted therapies. Antibodies directed to the tumor can interfere with the growth and the continued presence of tumors in a number of ways. Some target the tumor and mark it such that the immune system of the host is able to destroy the tumor cells. Some target signaling pathways that are linked to the cancerous state. Other interfere with a tumor cell’s capability to hide from or downregulate the hosts immune system against the tumor cell or environment hosting the tumor cell. Various other modes of action have been described.

Antibodies are a significant advancement over classic cancer treatment methods both in efficacy and in the reduction in the kind and severity of side effects. Relatively new is the development of multispecific antibodies. Such antibodies are typically designed to bind to multiple targets. A multispecific antibody may have an activity spectrum that is different from the simple combination of two or more monospecific antibodies with the respective binding properties of the multispecific antibody. That is, different mechanisms of action and results can be obtained from the use of a multispecific antibody targeting two or more antigens from the use of a combination of monospecific antibodies targeting each of those antigens. One example thereof is a T-cell engaging multispecific antibody. Such antibodies have, for example, a variable domain that binds CD3 or another T-cell activating antigen on the membrane of T-cells and a variable domain that binds a tumor antigen. Without being bound by theory it is believed that T-cell engaging antibodies bring/hold T-cells in the vicinity of the (tumor) target cell and through the activation of the T-cell induce/stimulate an immune response against the tumor.

Many of these treatments are still amenable to improvement. Aspects that can be improved are, for instance, diminishing the effects of multispecific antibodies on normal cells, which can lead to unwanted side effects, including higher toxicity or diminished tolerability in patients to the antibodies. Many tumor antigens are not strictly expressed on tumor cells. Indeed many of the tumor antigens are also expressed on non-tumor, also referred to herein as “normal”, cells. The ErbB family of proteins, for instance, are over-expressed and/or mutated in a variety of cancers but are also normally expressed on a variety of normal cells of an individual. Targeting such tumor antigens with an ablative antibody will typically affect normal, non-tumor cells and thereby at least potentially cause effects that are not associated with the tumor-attacking aspect of the antibody. Such target-specific side effects can, in severe cases, result in debilitating toxicity even death, and more commonly, in decreased quality of life as well as a decrease, interruption or discontinuation of the specific treatment. Targeting antibodies to EGFR, for instance, can lead to reactions that are most evident in tissues where EGFR is normally expressed to regulate a physiological function, such as in the skin. It is reported that patients treated with EGFR inhibitors can develop a papulopustular rash, dry skin, itching, and hair and periungual (the area that surrounds the nail) alterations (Lacouture 2006, nature reviews: cancer Vol 6, pp 803-812: doi:10.1038/nrc1970).

The invention provides means and methods for improving the efficacy and/or toxicity window of a multivalent, in particular a multispecific, antibody treatment. The treatment efficacy can be enhanced, the toxicity decreased, and tolerability increased, or each of these results, when compared to a similar dose of the multivalent antibody in the absence of the means and methods of the invention.

### SUMMARY OF THE INVENTION

The invention provides a composition comprising a multivalent antibody comprising a first variable domain that binds a first tumor antigen (TA1), a second variable domain that binds a second tumor antigen (TA2) and a third variable domain that binds an immune cell engaging antigen (IEA); and wherein the composition further comprises a second binding molecule that binds TA1 or TA2.

The combination of the multivalent antibody as described herein with the second binding molecule provides for a larger therapeutic window, in which off-target effects of the multivalent antibody are mitigated when administered in combination with the second binding molecule, as opposed to administration of the multivalent antibody alone.

A multivalent antibody of the invention may have any antibody format known in the art. Examples of antibody formats known in the art include, but are not limited to, those shown in FIG. 12 and disclosed in for example WO2019/190327. A multivalent antibody of the invention is a multispecific antibody.

An example of a multivalent antibody of the invention comprises a base antibody comprising a variable domain that binds an immune cell engaging antigen (IEA), preferably CD3, TCR-α chain or TCR-β chain, and a variable domain that binds TA2. The variable domain of the multivalent antibody that binds TA1 may be an additional variable domain that is linked to the variable domain that binds the immune cell engaging antigen (IEA) or that is linked to the variable domain that binds TA2. Another example of a multivalent antibody of the invention comprises a base antibody comprising a variable domain that binds an immune cell engaging antigen (IEA), preferably CD3, TCR-α chain or TCR-β chain, and a variable domain that binds TA1. The variable domain of the multivalent antibody that binds TA2 may be an additional variable domain that is linked to the variable domain that binds the immune cell engaging antigen (IEA) or that is linked to the variable domain that binds TA1. A further example of a multivalent antibody of the invention comprises a base antibody comprising a variable domain that binds to TA1 and a variable domain that binds to TA2. The variable domain of the multivalent antibody that binds the immune cell engaging antigen (IEA), preferably CD3, TCR-α chain or TCR-β chain, may be an additional variable domain that is linked to the variable domain that binds TA1 or that is linked to the variable domain that binds TA2.

A variable domain comprises at least one of a heavy chain variable or a light chain variable region, preferably at least a heavy chain variable region, more preferably both a heavy chain variable region and a light chain variable region.

For ease of reference, the variable domains on the multivalent, or multispecific, antibody can be referred to as domain 1, domain 2, and domain 3. Different heavy chain variable regions can be referred to by a different number such as VH1, VH2 and VH3. The invention therefore provides a composition or kit of parts comprising a multivalent antibody wherein the base antibody variable domains and the additional variable domain comprise heavy chain variable regions VH1, VH2 and VH3. In certain embodiments, the above described base antibody variable domain that binds to the immune cell engaging antigen (IEA) comprises heavy chain variable region VH2. In certain embodiments, the base antibody variable domain that binds to TA2 comprises heavy chain variable region VH3. In certain embodiments, the additional variable domain that binds to TA1 comprises heavy chain variable region VH1. In certain embodiments, the variable domain with VH1 is linked to the variable domain with VH2, suitably by means of a linker. In certain embodiments, the variable domain of the base antibody that binds to the immune cell engaging antigen (IEA) comprises heavy chain variable region VH2, the base antibody variable domain that binds to TA2 comprises heavy chain variable region VH3, the additional variable domain that binds to TA1 comprises heavy chain variable region VH1, and the variable domain with VH1 is linked to the variable domain with VH2, suitably by means of a linker. One example of a suitable format of a multivalent antibody is provided as a schematic representation in FIG. 1. Others are set out herein, including in FIG. 12, and provided in WO2019/190327, incorporated by reference. Different light chain variable regions can also be referred to by a different number such as VL1, VL2 and VL3. The multivalent, or multispecific, antibody used in the present invention may comprise a common light chain with three different heavy chain variable regions, a common heavy chain with three different light chain variable regions, or three different variable domains, such as domains each comprising heavy and light chain variable regions that differ from each other.

The second binding molecule of the invention is a monospecific binding molecule which binds TA1 or TA2. The second binding molecule can be any binding molecule with specificity for TA1 or TA2, including, but not limited to an antibody or fragment or variant thereof that maintains the binding specificity of said antibody, or a structure comprising said fragment. The second binding molecule is preferably a full length antibody, a Fab, a modified Fab, or a scFv.

The second binding molecule binds TA1 or TA2, thereby preventing or competing with binding of TA1 or TA2 by the multivalent antibody. This prevents or reduces killing of the cell when it expresses TA1 but does not express TA2, or when it expresses TA2 but does not express TA1. When a cell expresses both TA1 and TA2, the multivalent antibody binds to TA2 and thereby has an enhanced competition advantage over the second binding molecule for binding to TA1, or the multivalent antibody binds to TA1 and thereby has an enhanced competition advantage over the second binding molecule for binding to TA2. As such, the multivalent antibody is believed to exhibit an enhanced effect on cells expressing both TA1 and TA2 as compared to cells expressing either TA1 or TA2 alone.

The invention further provides a kit of parts comprising a multivalent antibody of the invention and a second binding molecule of the invention.

The invention further provides a therapeutic composition comprising a multivalent antibody of the invention and a second binding molecule of the invention.

The invention further provides a pharmaceutical composition comprising a multivalent antibody of the invention, a second binding molecule of the invention, and a pharmaceutically acceptable carrier and/or diluent. The multivalent antibody and second binding molecule of the invention may be formulated and/or administered together or separately.

The invention further provides a combination of a multivalent antibody and a second binding molecule of the invention for mitigating or reducing binding of the multivalent antibody to non-tumor cells, and/or for mitigating or reducing cell killing of non-tumor cells induced by the multivalent antibody. The invention further provides a combination of a multivalent antibody and a second binding molecule of the invention for use as a medicament. The invention further provides a combination of a multivalent antibody and a second binding molecule of the invention for use in the treatment of a subject in need thereof, in particular for use in the treatment of cancer. The multivalent antibody and second binding molecule may be administered simultaneously, or sequentially with the second binding molecule preceding or coming after the administration of the multivalent antibody.

The invention further provides a composition comprising a multivalent antibody and a second binding molecule of the invention for mitigating or reducing binding of the multivalent antibody to non-tumor cells, and/or for mitigating or reducing cell killing of non-tumor cells induced by the multivalent antibody. The invention further provides a composition comprising a multivalent antibody and a second binding molecule of the invention for use as a medicament. The invention further provides a composition comprising a multivalent antibody and a second binding molecule of the invention for use in the treatment of a subject in need thereof, in particular for use in the treatment of cancer.

A composition comprising a multivalent antibody comprising a first variable domain that binds a first tumor antigen (TA1), a second variable domain that binds a second tumor antigen (TA2) and a third variable domain that binds an immune cell engaging antigen (IEA); and wherein the composition further comprises a second binding molecule that binds TA1 or TA2, as described in the means, methods, uses in any form or combination of the present invention is preferably a therapeutic composition.

The invention further provides a therapeutic composition comprising a multivalent antibody and a second binding molecule of the invention for mitigating or reducing binding of the multivalent antibody to non-tumor cells, and/or for mitigating or reducing cell killing of non-tumor cells induced by the multivalent antibody. The invention further provides a therapeutic composition comprising a multivalent antibody and a second binding molecule of the invention for use as a medicament. The invention further provides a therapeutic composition comprising a multivalent antibody and a second binding molecule of the invention for use in the treatment of a subject in need thereof, in particular for use in the treatment of cancer.

The invention further provides a kit of parts comprising a multivalent antibody and a second binding molecule of the invention for mitigating or reducing binding of the multivalent antibody to non-tumor cells, and/or for mitigating or reducing cell killing of non-tumor cells induced by the multivalent antibody. The invention further provides a kit of parts comprising a multivalent antibody and a second binding molecule of the invention for use as a medicament. The invention further provides a kit of parts comprising a multivalent antibody and a second binding molecule of the invention for use in the treatment of a subject in need thereof, in particular for use in the treatment of cancer. The multivalent antibody and second binding molecule may be administered simultaneously, or sequentially with the second binding molecule preceding or coming after the administration of the multivalent antibody.

The invention further provides a method for mitigating or reducing binding of a multivalent antibody of the invention to non-tumor cells, and/or for mitigating or reducing cell killing of non-tumor cells induced by the multivalent antibody, wherein the method comprises using a second binding molecule as described herein in conjunction with the multivalent antibody.

The invention further provides a method of treatment of cancer, wherein the method comprises administering to the subject in need thereof a multivalent antibody of the invention, and additionally administering to the subject a second binding molecule of the invention.

### BRIEF DESCRIPTION OF THE DRAWINGS

It should be noted that features and aspects of the invention other than those exemplified below are apparent from the detailed description, taken in conjunction with the accompanying drawings, which illustrate, by way of example, the features in accordance with embodiments of the invention. Each of the figures provided is exemplary and is not intended to limit the scope of the invention provided, which are defined by the claims, aspects and the full extent of the present disclosure, which describe and enable the invention set out herein.

For ease of reference, when describing the multivalent antibody of the invention herein, the following format is used TA1=IEAxTA2 representing a tumor associated antigen 1 binding domain (TA1), linker (=), immune cell engaging antigen binding domain (IEA) dimerized (x) with a tumor associated antigen 2 binding domain (TA2), such that TA1=IEA constitutes the “long arm”, while the x denotes the dimerization, followed by TA2 which designates the “short arm” of the multivalent antibody. Where the multivalent antibody comprises a common light chain, the corresponding VH regions are as follows: TA1(VH1)=IEA(VH2)xTA2(VH3).

FIG. 1. Schematic representation of an example of a multivalent antibody. VH is heavy chain variable region, CH is heavy chain constant region, CL is light chain constant region, VL is light chain variable region. In this particular embodiment a common light chain is employed in each of the binding domains. The light chain, or VL, can also be common for one or more of the binding domains and different for another binding domain or other binding domains. In this particular embodiment, the additional binding domain with VH1 that binds to TA1 comprises a CH1 and CL domain. The multivalent antibody may for instance also lack one or both of these domains, or the CH1 and CL domains may be swapped. In this particular embodiment, the multivalent antibody comprises a linker between the CH1 domain of the additional binding domain with VH1 that binds to TA1 and the VH domain of the IEA binding domain with VH2. The linker may also be present between the CL domain of the additional binding domain with VH1 that binds to TA1 and the VL of the IEA binding domain with VH2, as an additional or as a single linker.

FIG. 2. Schematic representation of an example of a multivalent antibody with binding domains for PD-L1, EGFR and CD3. VH is heavy chain variable region, CH is heavy chain constant region, CL is light chain constant region, VL is light chain variable region. In this particular embodiment a common light chain is employed in each of the binding domains. The light chain, or VL, can also be common for one or more of the binding domains and different for another binding domain or other binding domains. In this particular embodiment, the additional binding domain that binds to PD-L1 comprises a CH1 and CL domain. The multivalent antibody may for instance also lack one or both of these domains, or the CH1 and CL domains may be swapped. In this particular embodiment, the multivalent antibody comprises a linker between the CH1 domain of the additional binding domain that binds to PD-L1 and the VH domain of the CD3 binding domain. The linker may also be present between the CL domain of the additional binding domain that binds to PD-L1 and the VL of the CD3 binding domain, as an additional or as a single linker.

FIG. 3. Amino acid sequence of a) a common light chain amino acid sequence; b) a common light chain variable region DNA sequence and translation (IGKV1-39/jk1); c) a common light chain constant region DNA sequence and translation; d) IGKV1-39/jk5 common light chain variable region amino acid sequence; e) V-region IGKV1-39 amino acid sequence; f) CDR1, CDR2 and CDR3 amino acid sequence of a common light chain.

FIG. 4. Exemplary IgG heavy chain nucleic acid and amino acid sequences suitable for the generation of bispecific molecules. a) CH1 region. b) hinge region. c) CH2 region. d) CH3 domain comprising variations L351K and T366K (KK). e) CH3 domain comprising variations L351D and L368E (DE).

FIG. 5. Panel A depicts normal, non-tumor, cells that are either PD-L1 positive and EGFR negative, or EGFR positive and PD-L1 negative. Such cells are not efficiently lysed by a trispecific PD-L1 = CD3 x EGFR antibody in the presence of a monospecific PD-L1binding molecule. The cross (X) through the arrow means no lysis or weak lysis.

The monospecific PD-L1 binding molecule outcompetes the trispecific antibody on PD-L1 positive and EGFR negative cells, for example, due to, bivalency of the PD-L1 binding molecule and/or a higher affinity of the PD-L1 binding molecule compared to the affinity of the trispecific antibody for PD-L1. The trispecific antibody binds EGFR positive and PD-L1 negative cells inducing no or relatively weak activity, because of, for example, the monovalent character of the binding.

However, with cells expressing both EGFR and PD-L1, such as tumor cells, the trispecific antibody docks to the cell via EGFR and the PD-L1 targeting arm has an enhanced competition advantage with respect to the monospecific anti-PD-L1 binding molecule (Panel B). The trispecific antibody has greater binding to such PD-L1 positive and EGFR positive cells, achieved via avidity through binding to both EGFR and PD-L1, as compared to the monospecific PD-L1 binding molecule. This may be further enhanced by using a high affinity of the EGFR targeting arm.

FIG. 6. FIG. 6 shows the results from a cytotoxicity study performed using BxPC3 cells co-cultured with human T cells. Two different PD-L1 = CD3 x EGFR trispecific antibodies were tested: one having a PD-L1 binding domain comprising SEQ ID NO: 38, a CD3 binding domain comprising SEQ ID NO: 8 and an EGFR binding domain comprising SEQ ID NO: 56; and the other having a PD-L1 binding domain comprising SEQ ID NO: 42, a CD3 binding domain comprising SEQ ID NO: 22 and an EGFR binding domain comprising SEQ ID NO: 56. The cell killing activity of the trispecific antibodies was tested in the presence of a monospecific bivalent PD-L1 antibody having PD-L1 binding domains comprising heavy chains having an amino acid sequence as set forth in SEQ ID NO: 46 (FIG. 6A) or a monospecific bivalent PD-L1 antibody having PD-L1 binding domains comprising heavy chains having an amino acid sequence set forth in SEQ ID NO: 47 (FIG. 6B). The monospecific PD-L1 antibodies were used at different concentrations: 20 nM, 2.05 nM, 0.205 nM, 0.0205 nM, 0.00205 nM, and 0 nM (left to right columns). The y-axis of each plot indicates the % target cell killing compared to a control sample not comprising an antibody. The x-axis of each plot indicates the amount in nanomolar (nM) of the respective trispecific antibody in the sample. The panels compare the activity of a PD-L1 = CD3 x EGFR trispecific antibody with a trispecific PD-L1 = CD3 x Mock control antibody. The mock variable domain has the heavy chain variable region of SEQ ID NO: 68 which together with the common light chain forms a Tetanus Toxoid binding variable domain (TT). The TT-variable domain does not have a binding partner in the various incubations and thus serves as a mock-domain.

FIG. 7. FIG. 7 shows the results from a cytotoxicity study performed using BxPC3 cells (top panel) or HTC116 cells (lower panel) co-cultured with human T cells. Three different PD-L1 = CD3 x EGFR trispecific antibodies were tested: one having a PD-L1 binding domain comprising SEQ ID NO: 38, a CD3 binding domain comprising SEQ ID NO: 8 and an EGFR binding domain comprising SEQ ID NO: 56 (left column); one having a PD-L1 binding domain comprising SEQ ID NO: 38, a CD3 binding domain comprising SEQ ID NO: 22 and an EGFR binding domain comprising SEQ ID NO: 56 (middle column), and one having a PD-L1 binding domain comprising SEQ ID NO: 42, a CD3 binding domain comprising SEQ ID NO: 22 and an EGFR binding domain comprising SEQ ID NO: 56 (right column). The cell killing activity of the trispecific antibodies was tested in the presence of a monospecific bivalent PD-L1 antibody having PD-L1 binding domains comprising heavy chains having an amino acid sequence as set forth in SEQ ID NO: 46 or a monospecific bivalent PD-L1 antibody having PD-L1 binding domains comprising heavy chains having an amino acid sequence as set forth in SEQ ID NO: 47. The monospecific PD-L1 antibody was used at a 10-fold concentration of the trispecific antibodies. The y-axis of each plot indicates the % target cell killing compared to a control sample not comprising a trispecific antibody. The x-axis of each plot indicates the amount in ng/ml of the respective trispecific antibody in the sample. The panels compare the activity of a PD-L1 = CD3 x EGFR trispecific antibody with a trispecific PD-L1 = CD3 x Mock control antibody and a trispecific Mock = CD3 x EGFR control antibody. The mock variable domain has the heavy chain variable region of SEQ ID NO: 68 which together with the common light chain forms a Tetanus Toxoid binding variable domain (TT). The TT-variable domain does not have a binding partner in the various incubations and thus serves as a mock-domain.

FIG. 8. PD-L1 = CD3 x EGFR trispecific antibody in combination with monospecific bivalent PD-L1 antibody in a cytotoxicity assay for determining T cell-mediated target cell killing using human T cells and BxPC3 cells. Two different PD-L1 = CD3 x EGFR trispecific antibodies were tested: one having a PD-L1 binding domain comprising SEQ ID NO: 38, a CD3 binding domain comprising SEQ ID NO: 8 and an EGFR binding domain comprising SEQ ID NO: 56 (left column); and one having a PD-L1 binding domain comprising SEQ ID NO: 42, a CD3 binding domain comprising SEQ ID NO: 22 and an EGFR binding domain comprising SEQ ID NO: 56 (right column).

The x-axis indicates the amount of the trispecific antibody in nM. The y-axis indicates the % cell killing relative to when no antibodies are added. The top row shows the cell killing activity of the trispecific PD-L1 = CD3 x EGFR antibody or mock control when no bivalent monospecific antibody is present (vehicle). The middle row shows the cell killing activity of the trispecific PD-L1 = CD3 x EGFR antibody or mock control when a bivalent monospecific antibody is added in an equal amount (ratio trispecific : monospecific is 1 : 1). The lower row shows the cell killing activity of the trispecific PD-L1 = CD3 x EGFR antibody or mock control when a bivalent monospecific antibody is added in a ten-fold excess (ratio trispecific : monospecific is 1 : 10). FIG. 8A shows the results when a bivalent monospecific antibody comprising heavy chains with SEQ ID NO: 46 is added, and FIG. 8B shows the results when a bivalent monospecific antibody comprising heavy chains with SEQ ID NO: 51 is added.

FIG. 9. PD-L1 = CD3 x EGFR trispecific antibody in combination with monospecific bivalent PD-L1 antibody in a cytotoxicity assay for determining T cell -mediated target cell killing using a human T cells and BxPC3 cells. Three different PD-L1 = CD3 x EGFR trispecific antibodies were tested: one having a PD-L1 binding domain comprising SEQ ID NO: 38, a CD3 binding domain comprising SEQ ID NO: 8 and an EGFR binding domain comprising SEQ ID NO: 56 (left column); one having a PD-L1 binding domain comprising SEQ ID NO: 38, a CD3 binding domain comprising SEQ ID NO: 22 and an EGFR binding domain comprising SEQ ID NO: 56 (middle column); and one having a PD-L1 binding domain comprising SEQ ID NO: 42, a CD3 binding domain comprising SEQ ID NO: 22 and an EGFR binding domain comprising SEQ ID NO: 56 (right column). The bivalent monospecific PD-L1 antibody used comprises heavy chains having an amino acid sequence as set forth in SEQ ID NO: 46.

The x-axis indicates the amount of the trispecific antibody in nM. The y-axis indicates the % cell killing relative to when no antibodies are added. The top row shows the cell killing activity of the trispecific PD-L1 = CD3 x EGFR antibody or mock control when no bivalent monospecific antibody is present (vehicle). The middle row shows the cell killing activity of the trispecific PD-L1 = CD3 x EGFR antibody or mock control when a bivalent monospecific antibody is added in an equal amount (ratio trispecific : monospecific is 1 : 1). The lower row shows the cell killing activity of the trispecific PD-L1 = CD3 x EGFR antibody or mock control when a bivalent monospecific antibody is added in a ten-fold excess (ratio trispecific : monospecific is 1 : 10).

FIG. 10. Map of Vector MV3032.

FIG. 11: Map of Vector MV1625.

FIG. 12: Schematic representation of examples of suitable multivalent antibody formats. These multivalent antibody formats may comprise further additional binding domains.

FIG. 12A shows examples of multivalent antibody formats comprising an Fc region. BD1, BD2, and BD3 are binding domains 1, 2, and 3. Certain binding domains in these examples are indicated as Fab domains; however, other types of domains may also be used, such as for instance a single domain antibody, VHH, Fv, VHH2, scFv, diabody, CODV, etc., as well as combinations thereof. Certain binding domains in these examples are indicated as scFv domains; however, other types of domains may also be used, such as for instance a single domain antibody, VHH, Fv, VHH2, Fab, diabody, CODV, etc., as well as combinations thereof. One or more of the binding domains may also be linked to CH2 or engineered in the CH1, CH2, and/or CH3 region. The multivalent antibody formats may comprise any type of heavy chain and light chain, including a common heavy chain, a common light chain, orthogonal heavy chains, and orthogonal HC:LC. The position and or nature of the linker in the multivalent antibody formats may be varied, in accordance with what is known in the art.

FIG. 12B shows additional examples of multivalent antibody formats including: V domain, Fv and Fab-based multispecific antibodies (VHH3, triplebody, tandem Fab3), Fv-based IgG multispecific antibodies (CODV-Fab TsAb, scFv-IgG TsAb), Fab-based IgG multispecific antibodies (orthoTsAb), and CrossMab 2:1 TCB.

### DETAILED DESCRIPTION OF THE INVENTION

In order that the present description may be more readily understood, certain terms are first defined. Additional definitions are set forth throughout the detailed description. Unless stated otherwise, all technical and scientific terms used herein have the same meaning as commonly understood by one of ordinary skill in the art, and conventional methods of immunology, protein chemistry, biochemistry, recombinant DNA techniques and pharmacology are employed.

The articles “a” and “an” are used herein to refer to one or more than one (i.e. to one or at least one) of the grammatical object of the article.

Throughout the present specification and the accompanying claims and aspects, the words “comprise”, “include” and “having” and variations such as “comprises”, “comprising”, “includes” and “including” are to be interpreted inclusively. That is, these words are intended to convey the possible inclusion of other elements or integers not specifically recited, where the context allows.

The term “binding domain” as used herein means a proteinaceous molecule comprising a variable domain or may comprise a variable domain or that shares sequence homology with the variable domain. Non-limiting examples of binding domains comprising a variable domain are an Fv domain, a Fab domain and a modified Fab domain. Typical variability is found in three superficial-loop forming regions in the VH and VL domains, which are the complementarity determining regions or CDRs. The term “antibody” as used herein means a proteinaceous molecule belonging to the immunoglobulin class of proteins, containing one or more domains that bind an epitope on an antigen, where such domains are or are derived from or share sequence homology with the variable domain of an antibody. Antibodies are typically made of basic structural units-each with two heavy chains and two light chains. Antibodies for therapeutic use are preferably as close to natural antibodies of the subject to be treated as possible (for instance human antibodies for human subjects). An antibody according to the present invention is not limited to any particular format or method of producing it.

A “base antibody” or “base antibody portion” comprises two binding domains. It preferably consists of four polypeptides- two heavy chains and two light chains joined to form a “Y” shaped molecule. The base of the Y contains the multimerizing domains pairing the heavy chains, typically, CH3 and the CH2 domains. The two branches of the Y contain the two CH1 domains linked to the two variable domains. One of the CH3 sequences has one part of a compatible heterodimerization domain and the other CH3 sequence has the complementary part of the heterodimerization domain.

In one embodiment, the base antibody comprises two binding domains each comprising a heavy chain variable region, CH1, light chain variable region, and CL; each binding domain being associated with their CH1 region to a hinge and Fc region.

Antibody binding has different qualities including specificity, affinity, and avidity. The specificity determines which antigen or epitope thereof is specifically bound by the binding domain. The affinity is a measure for the strength of binding to a particular antigen or epitope. It is convenient to note here that the ‘specificity’ of an antibody refers to its selectivity for a particular antigen, whereas ‘affinity’ refers to the strength of the interaction between the antibody’s antigen binding site and the epitope it binds.

Thus, the “binding specificity” as used herein refers to the ability of an individual antibody binding site to react with an antigenic determinant. Typically, the binding site of the antibody of the invention is located in the variable domains of the Fab domains and is constructed from a hypervariable region of a heavy and/or light chain.

“Affinity” is the strength of the interaction between a single antigen-binding site and its antigen. A single antigen-binding site of an antibody of the invention for an antigen may be expressed in terms of the dissociation constant (k_{d}).

“Avidity” refers to the accumulated strength of the interaction between a bivalent or multivalent binding molecule and its antigen(s). Avidity is determined by the combined affinities of the multiple antigen-binding sites, and is dependent on the expression levels of each antigen on the target cell. The ability of bivalent or multivalent binding molecule to display avidity binding is depending on the capacity of the bivalent or multivalent binding molecules to bind their antigen simultaneously, referred to as cross-binding capacity.

An “epitope” or “antigenic determinant” is a site on an antigen to which an immunoglobulin or antibody specifically binds. Epitopes can be formed from contiguous amino acids or noncontiguous amino acids juxtaposed by tertiary folding of a protein (so-called linear and conformational epitopes, respectively). Epitopes formed from contiguous, linear amino acids are typically retained on exposure to denaturing solvents, whereas epitopes formed by tertiary folding, conformation are typically lost on treatment with denaturing solvents. An epitope may typically include 3, 4, 5, 6, 7, 8, 9, 10, 11, 12, 13, 14 or 15 amino acids in a unique spatial conformation.

The term “heavy chain” or “immunoglobulin heavy chain” includes an immunoglobulin heavy chain constant region sequence from any organism, and unless otherwise specified includes a heavy chain variable domain. The term heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a CH1 domain, a hinge, a CH2 domain, and a CH3 domain. A functional fragment of a heavy chain includes a fragment that is capable of specifically recognizing an antigen and that comprises at least one CDR.

The term “light chain” includes an immunoglobulin light chain variable domain, or VL (or functional fragment thereof); and an immunoglobulin constant domain, or CL (or functional fragment thereof) sequence from any organism.

Unless otherwise specified, the term light chain may include a light chain selected from a human kappa, lambda, and a combination thereof. Light chain variable (VL) domains typically include three light chain CDRs and four framework (FR) regions, unless otherwise specified. Generally, a full-length light chain includes, from N-terminus to C-terminus, a VL domain that includes FR1-CDR1-FR2-CDR2-FR3-CDR3-FR4, and a light chain constant domain. Light chains that can be used with this invention include those, e.g., that do not selectively bind an epitope selectively bound by the heavy chains.

Suitable light chains for use in a multivalent antibody invention include a common light chain, such as those that can be identified by screening for the most commonly employed light chains in existing antibody libraries (wet libraries or in silico), where the light chains do not substantially interfere with the affinity and/or selectivity of the epitope-binding domains of the heavy chains, but are also suitable to pair with an array of heavy chains. For example, a suitable light chain includes one from a transgenic animal, such as a transgenic rodent, comprising the common light chain integrated into its genome and which can be used to generate large panels of common light chain antibodies having diversity at the heavy chain upon exposure to an antigen (WO2009/157771). The common light chain that is part of the multivalent antibody can also be used as the light chain of the second antibody.

The term “common light chain” according to the invention refers to light chains which may be identical or have some amino acid sequence differences while the binding specificity of the an antibody of the invention is not affected, i.e. the differences do not materially influence the formation of functional binding regions.

It is for instance possible within the scope of the definition of common chains as used herein, to prepare or find variable chains that are not identical but still functionally equivalent, e.g., by introducing and testing conservative amino acid changes, changes of amino acids in regions that do not or only partly contribute to binding specificity when paired with a cognate chain, and the like. Such variants are thus also capable of binding different cognate chains and forming functional antigen binding domains. The term ‘common light chain’ as used herein thus refers to light chains which may be identical or have some amino acid sequence differences while retaining the binding specificity of the resulting antibody after pairing with a heavy chain. A combination of a certain common light chain and such functionally equivalent variants is encompassed within the term “common light chain”.

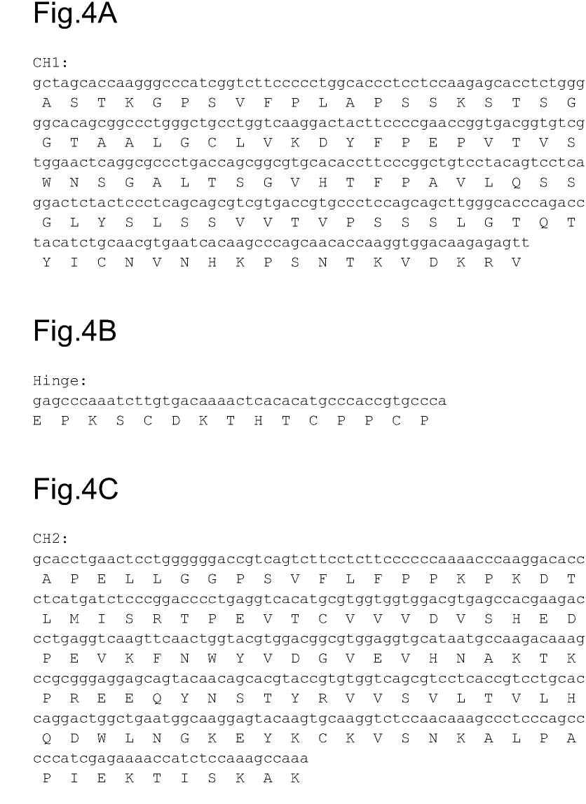

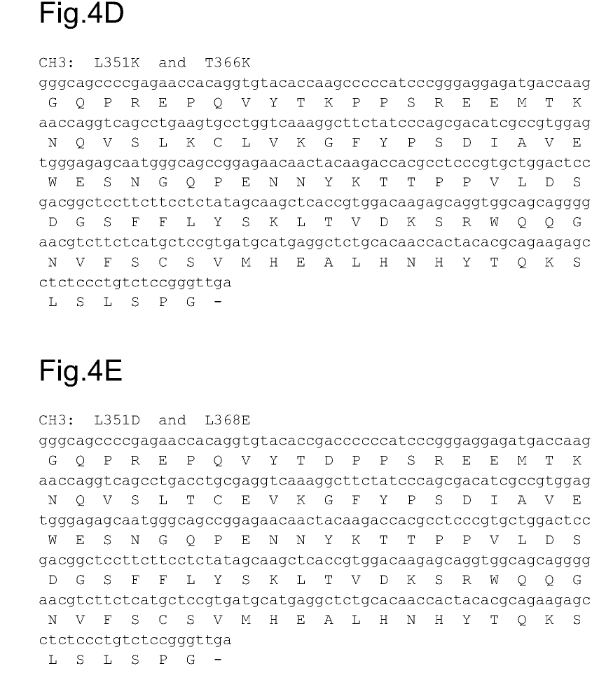

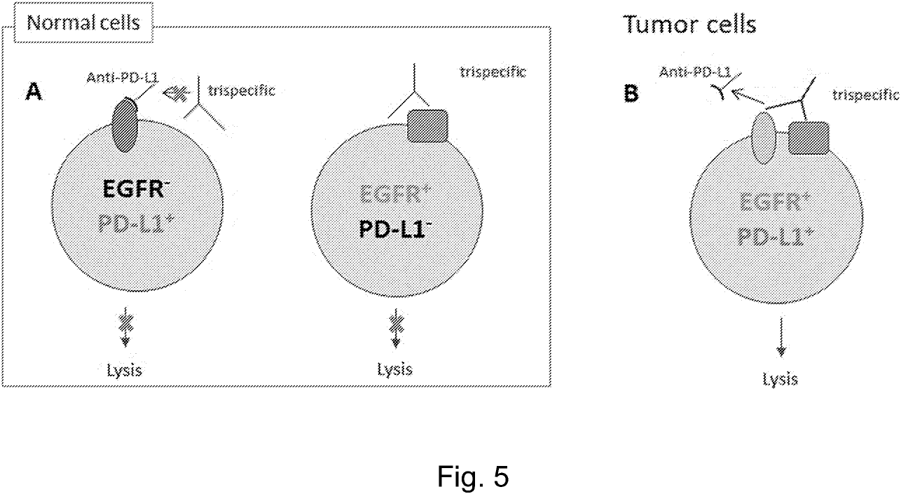

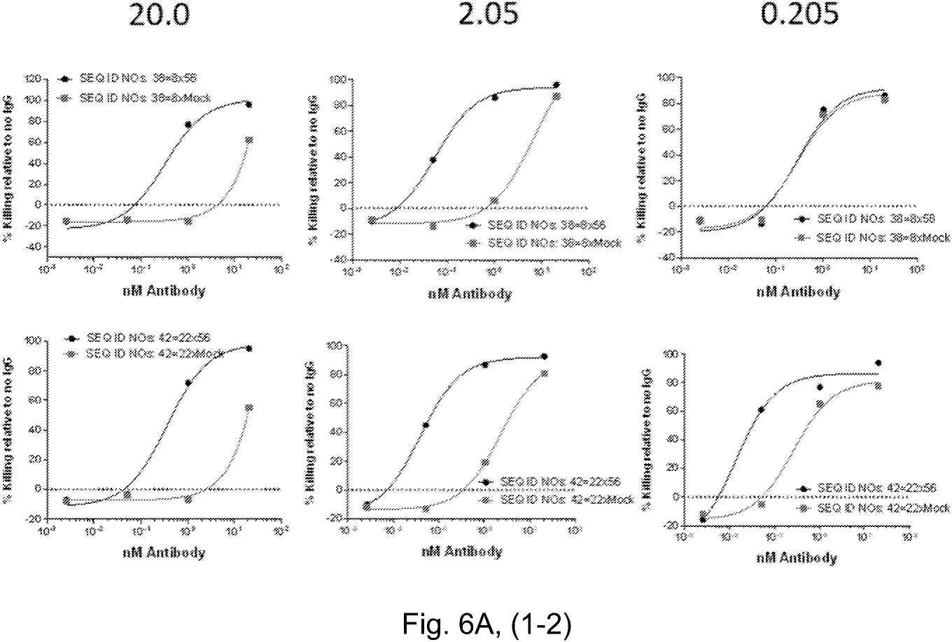

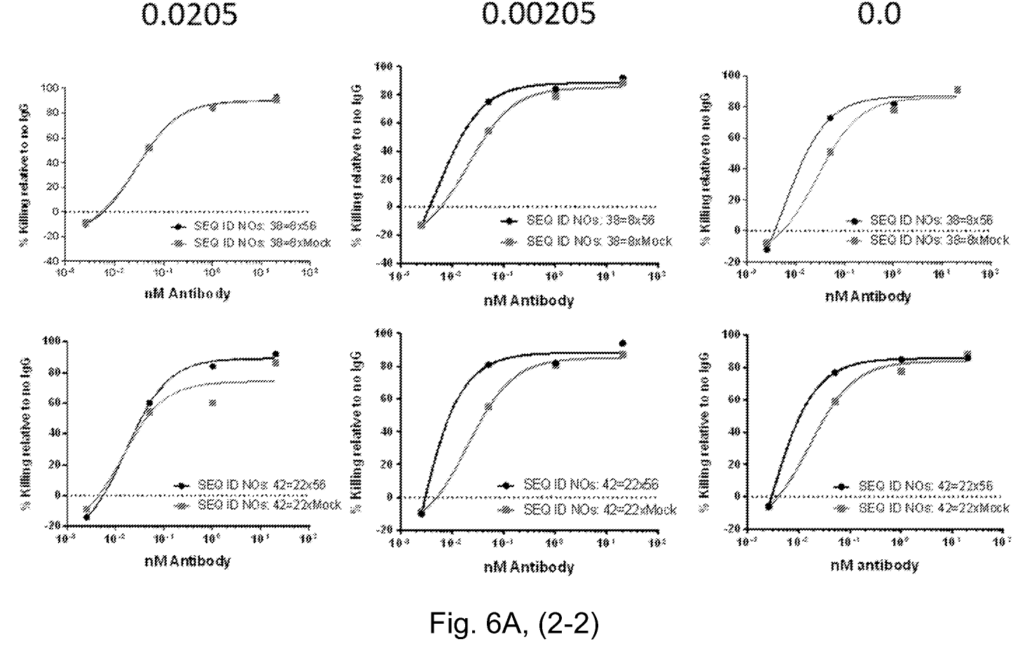

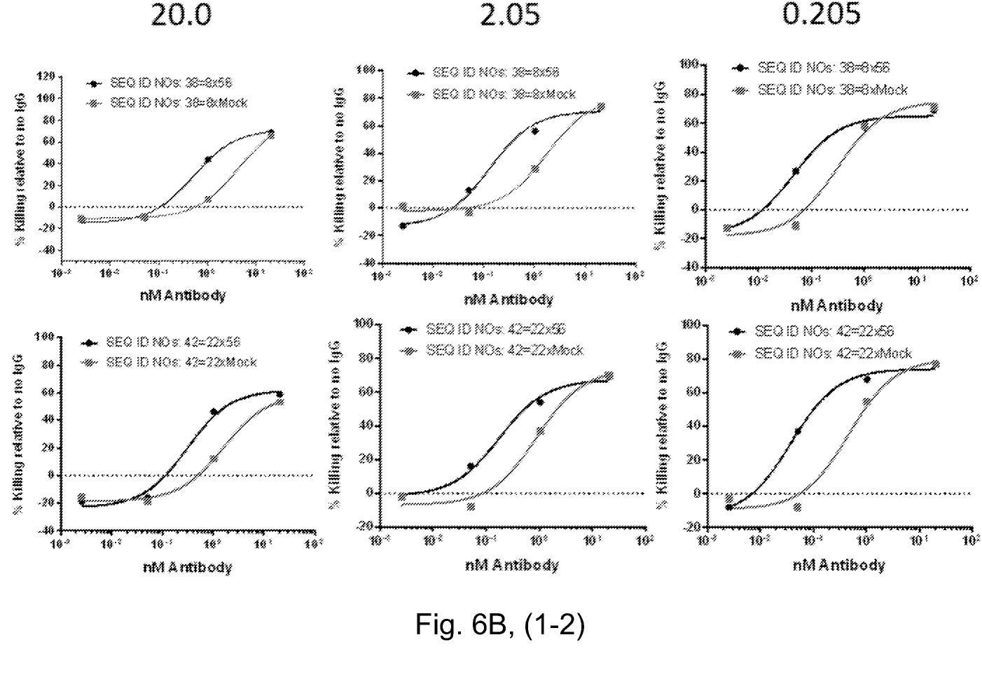

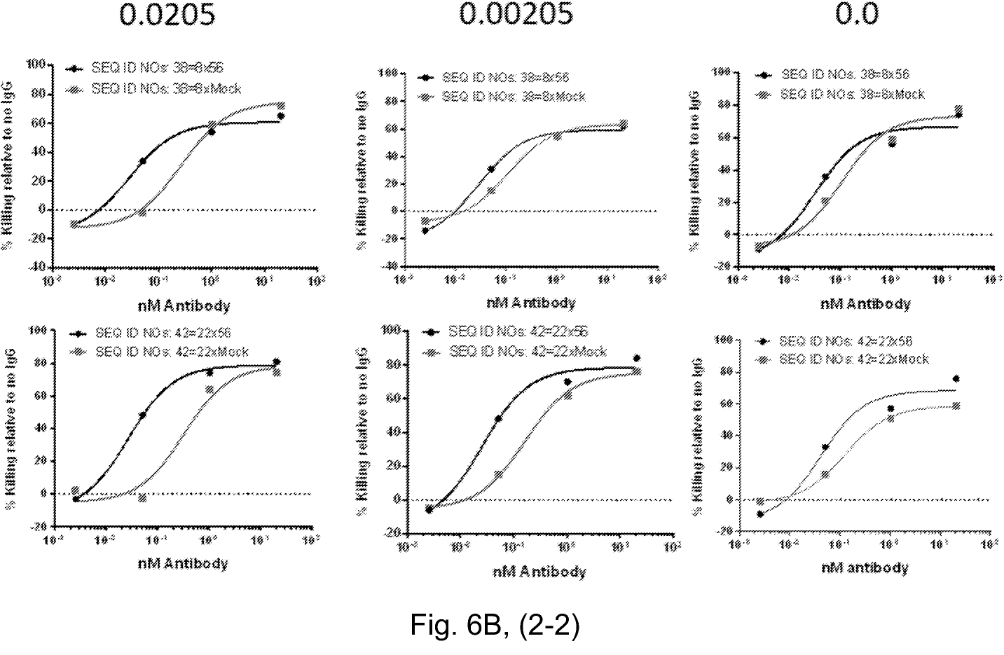

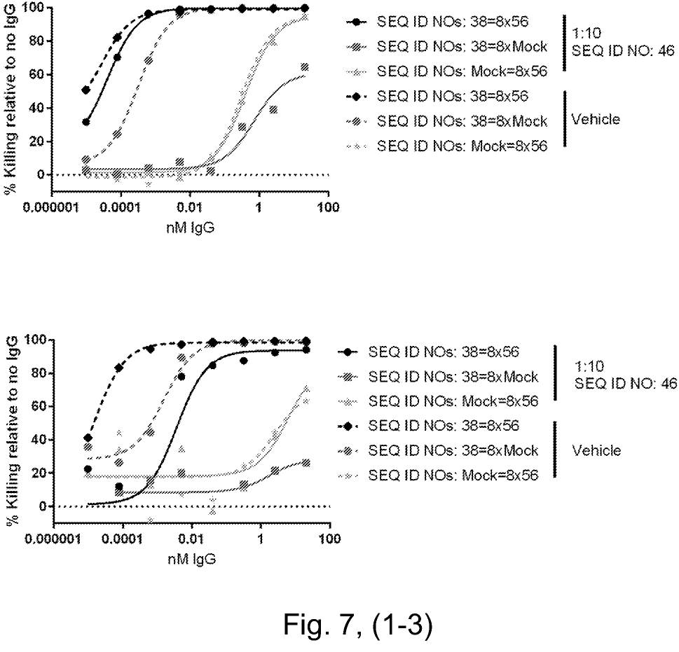

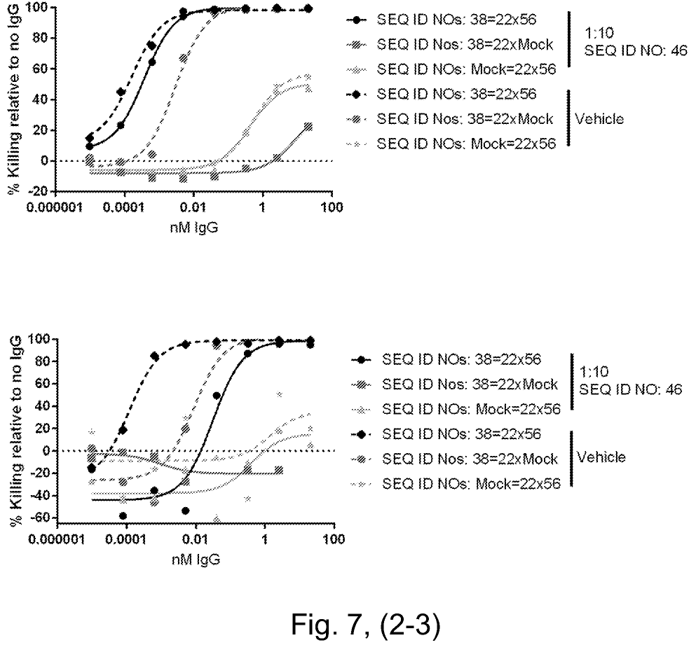

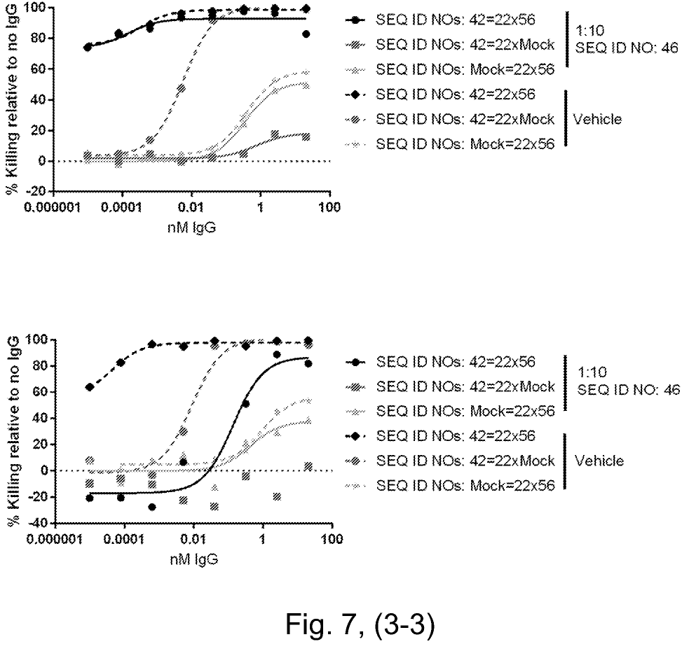

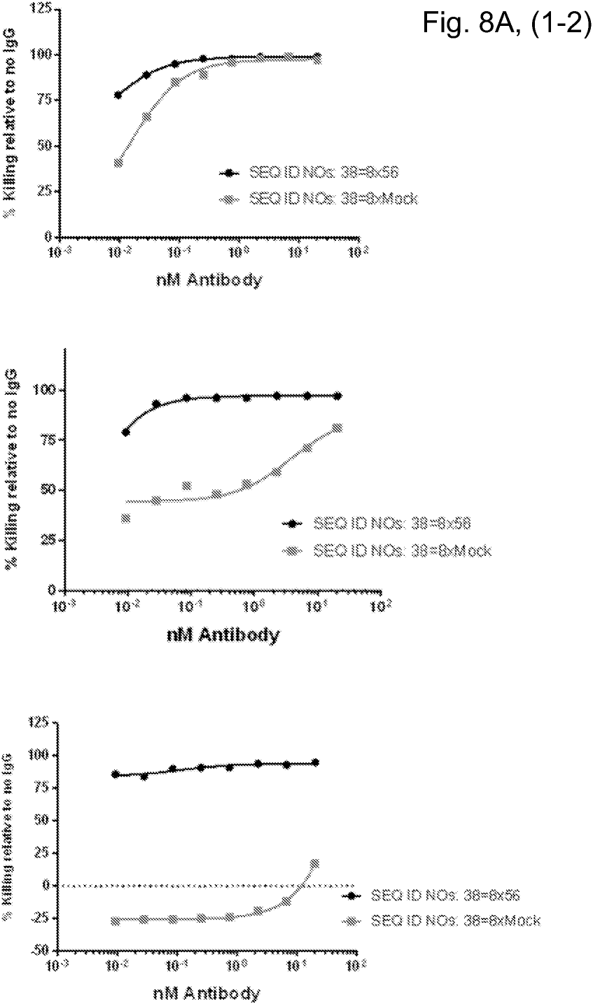

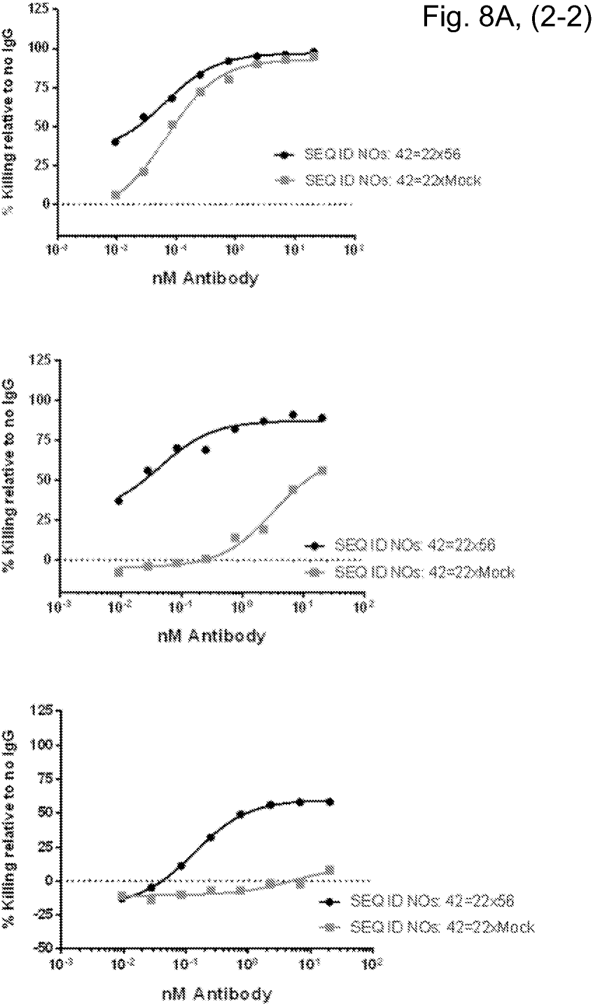

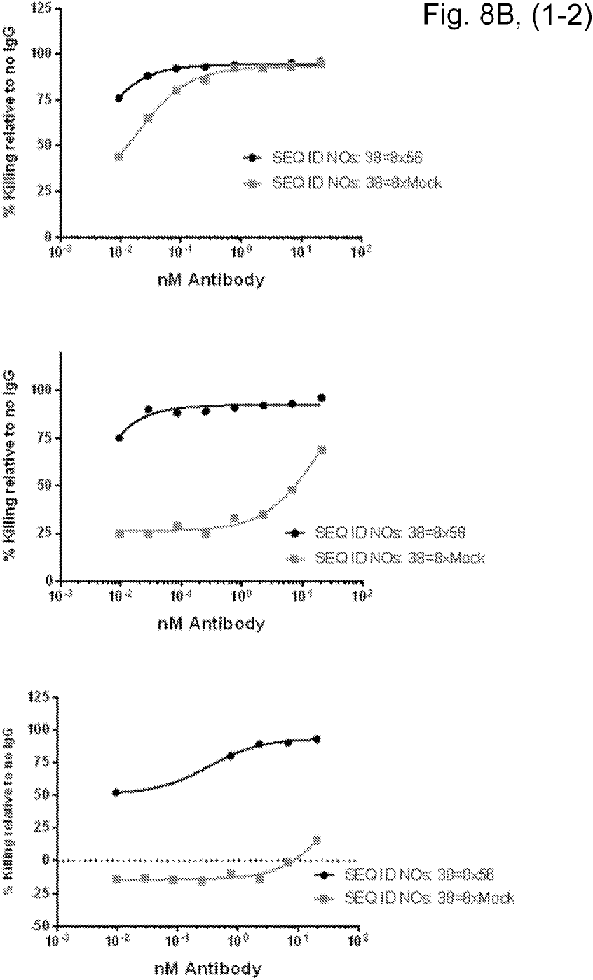

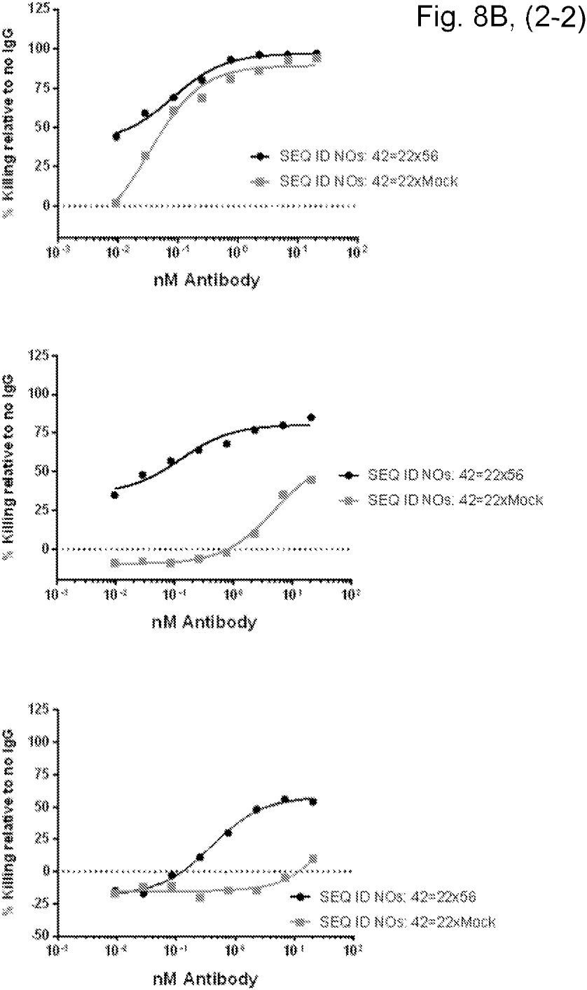

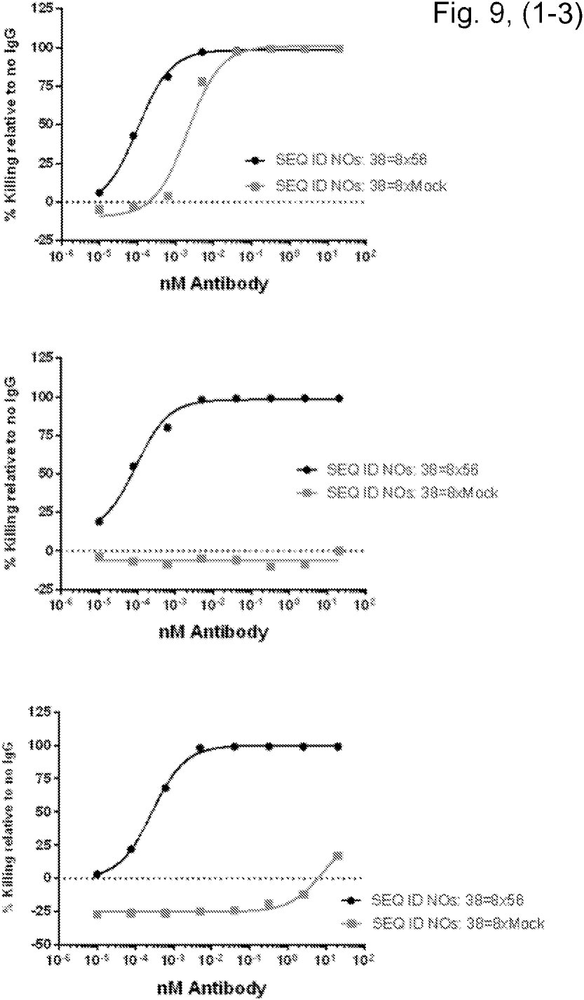

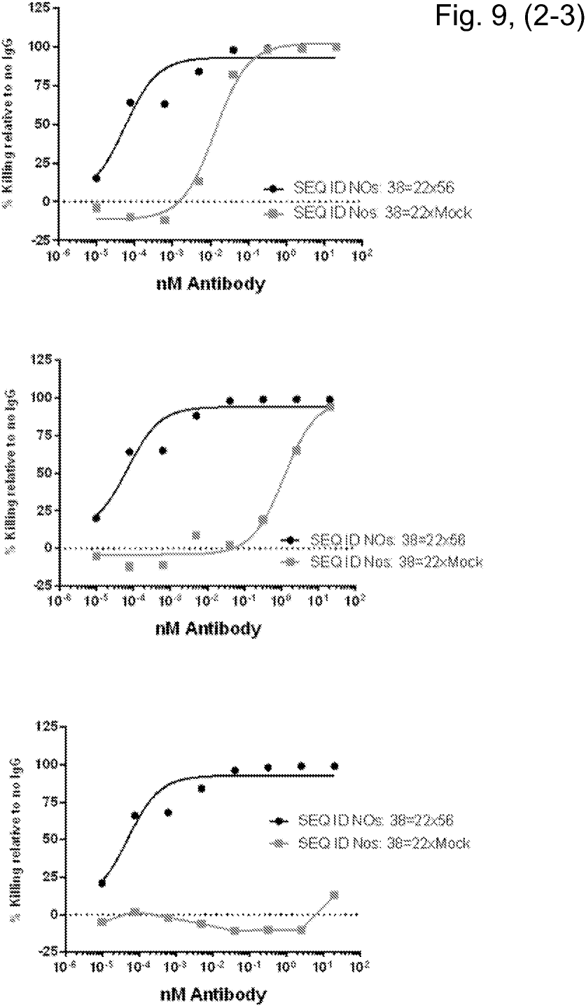

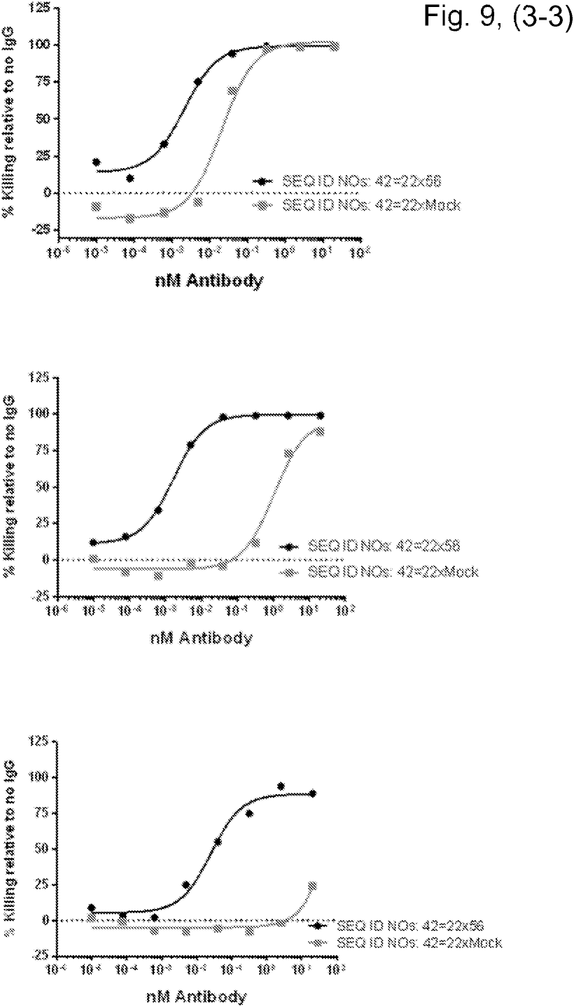

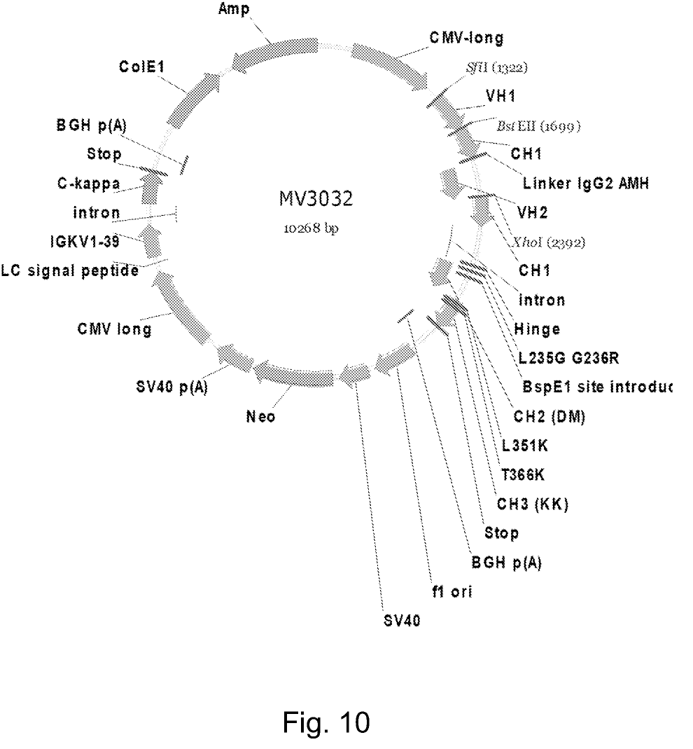

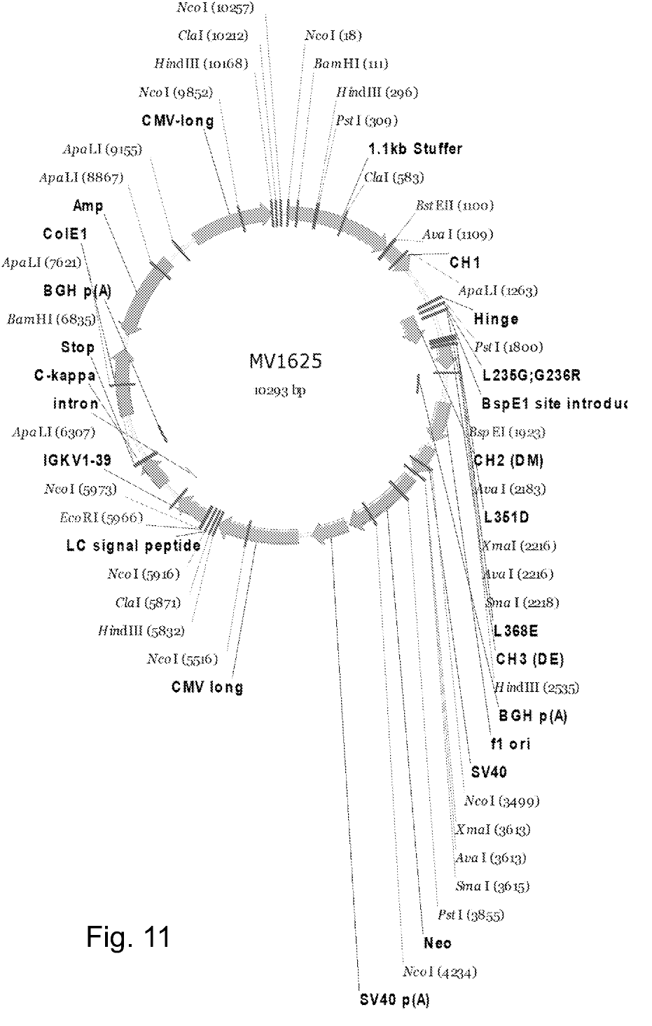

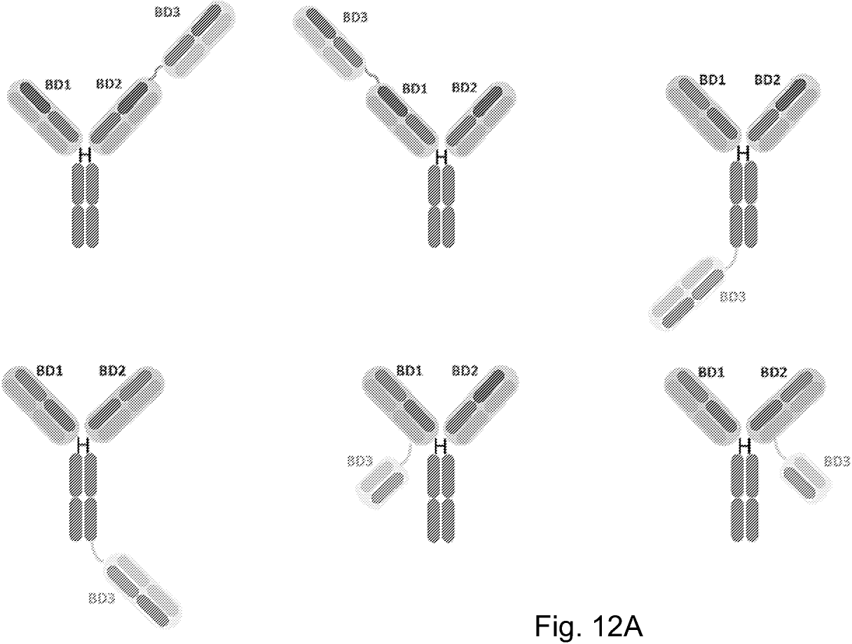

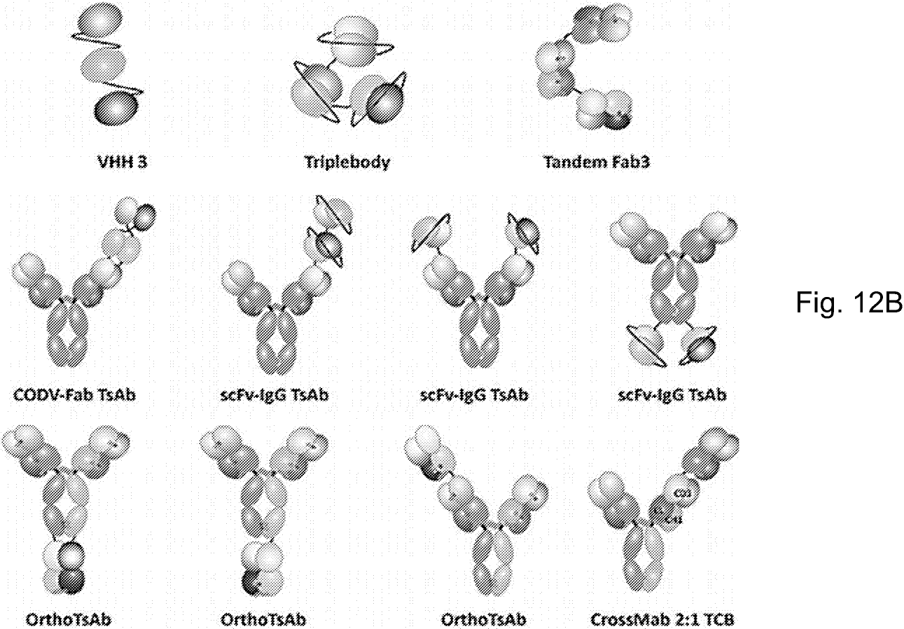

A preferred common light chain is designated IgV_{K}1-39*01/IGJ_{K}1*01. IgV_{K}1-39 is short for Immunoglobulin Variable Kappa 1-39 Gene. The gene is also known as Immunoglobulin Kappa Variable 1-39; IGKV139; IGKV1-39. External Ids for the gene are HGNC: 5740; Entrez Gene: 28930; Ensembl: ENSG00000242371. A preferred amino acid sequence for IgV_{K}1-39 is given in FIG. 4. This lists the sequence of the V-region. The V-region can be combined with one of five J-regions. FIG. 4 describes two preferred sequences for IgV_{K}1-39 in combination with a J-region. The joined sequences are indicated as IGKV1-39/jk1 and IGKV1-39/jk5; alternative names are IgV_{K}1-39*01/IGJ_{K}1*01 or IgV_{K}1-39*01/IGJ_{K}5*01 (nomenclature according to the IMGT database worldwide web at imgt.org).

Those of skill in the art will recognize that “common” also refers to functional equivalents of the light chain of which the amino acid sequence is not identical. Many variants of said light chain exist wherein mutations (deletions, substitutions, additions) are present that do not materially influence the formation of functional binding regions.

A “Fv domain” means a binding domain comprising a variable domain having a heavy chain variable region (VH) and a light chain variable region (VL).

A “Fab domain” means a binding domain comprising a variable region, typically a binding domain comprising a paired heavy chain variable region and light chain variable region. A Fab domain may comprise constant region domains, including a CH1 and a VH domain paired with a constant light domain (CL) and VL domain. Such pairing may take place, for example, as covalent linkage via a disulfide bridge at the CH1 and CL domains.

A “modified Fab domain” means a binding domain comprising a CH1 and a VH domain, wherein the VH is paired with a VL domain and no CL domain is present. Alternatively, a modified Fab domain is a binding domain comprising a CL and a VL domain, wherein the VL is paired with a VH domain and no CH1 domain is present. In order that the CH1 or CL region can be present in a non-paired form, it may be necessary to remove or reduce the lengths of regions of hydrophobicity. CH1 regions from species of animal that naturally express single-chain antibodies, for example from a camelid animal, such as a llama or a camel, or from a shark may be used. Other examples of a modified Fab domain include a Fab comprising a constant region, CH1 or CL, which is not paired with its cognate region and/or a variable region VH or VL, is present, which is not paired with its cognate region; and a Fab wherein the VH is swapped with the VL, wherein one polypeptide of a pair comprises VL-CH1 and the other polypeptide comprises VH-CL.

The term “immune effector cell” or “effector cell” as used herein refers to a cell within the natural repertoire of cells in the mammalian immune system which can be activated to affect the viability of a target cell. Immune effector cells include cells of the lymphoid lineage such as natural killer (NK) cells, T cells including cytotoxic T cells, or B cells, but also cells of the myeloid lineage can be regarded as immune effector cells, such as monocytes or macrophages, dendritic cells and neutrophilic granulocytes. Said effector cell is preferably an NK cell, a T cell, a B cell, a monocyte, a macrophage, a dendritic cell or a neutrophilic granulocyte.

The term “immune cell engaging antigen” as used herein refers to a molecule or moiety expressed on the cell membrane of said immune effector cell, and which when bound to its ligand or an activating antibody of the present invention results in activation, stimulation or co-stimulation of the immune cell, non-limiting examples of such antigens to be targeted include CD2, CD3, CD137, CD28, OX40, CD5, CD16, CD16A.

“Percent (%) identity” as referring to nucleic acid or amino acid sequences herein is defined as the percentage of residues in a candidate sequence that are identical with the residues in a selected sequence, after aligning the sequences for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/based or amino acids. The sequence identity is the percentage of identical matches between the two sequences over the reported aligned region.

A comparison of sequences and determination of percentage of sequence identity between two sequences can be accomplished using a mathematical algorithm. The skilled person will be aware of the fact that several different computer programs are available to align two sequences and determine the identity between two sequences (Kruskal, J. B. (1983) An overview of sequence comparison In D. Sankoff and J. B. Kruskal, (ed.), Time warps, string edits and macromolecules: the theory and practice of sequence comparison, pp. 1 -44 Addison Wesley). The percent sequence identity between two amino acid sequences or nucleic acid sequences may be determined using the Needleman and Wunsch algorithm for the alignment of two sequences. (Needleman, S. B. and Wunsch, C. D. (1970) J. Mol. Biol. 48, 443-453). The Needleman-Wunsch algorithm has been implemented in the computer program NEEDLE. For the purpose of this invention the NEEDLE program from the EMBOSS package is used to determine percent identity of amino acid and nucleic acid sequences (version 2.8.0 or higher, EMBOSS: The European Molecular Biology Open Software Suite (2000) Rice, P. LongdenJ. and Bleasby, A. Trends in Genetics 16, (6) pp276- 277, http://emboss.bioinformatics.nl/). For protein sequences, EBLOSUM62 is used for the substitution matrix. For DNA sequences, DNAFULL is used. The parameters used are a gap-open penalty of 10 and a gap extension penalty of 0.5.

After alignment by the program NEEDLE as described above the percentage of sequence identity between a query sequence and a sequence of the invention is calculated as follows: Number of corresponding positions in the alignment showing an identical amino acid or identical nucleotide in both sequences divided by the total length of the alignment after subtraction of the total number of gaps in the alignment.

Herein, the term “connected” or “linked” refers to domains which are joined to each other by way of peptide bonds at the primary amino acid sequence. For example, a heavy chain of a base antibody portion comprising VH-CH1-CH2-CH3 may be connected to a heavy chain of an additional binding domain VH-CH1 (or an additional binding domain to an additional binding domain) via a linker (connecting the heavy chain of the additional binding domain at the CH1 to the VH region of the base antibody portion), which together constitutes one polypeptide chain. Similarly, a CH1 domain may be connected to a variable heavy region and a CL domain may be connected to a variable light region. The antibody domains may also be “connected” by means not requiring a linker, such as for instance as part of a single polypeptide.

“Pairing” refers to interactions between the polypeptides constituting a multivalent antibody of the invention such that they may multimerize. For example, an additional binding domain may comprise a heavy chain region (VH-CH1) paired to a light chain region (VL-CL), where the CH1 and CL pair to form said binding domain. As described herein, pairing of antibody domains (e.g., heavy and light) occurs due to noncovalent interactions and also via disulfide bonds, and can be engineered through techniques disclosed herein and by methods known in the art. Such noncovalent interactions typically occur in an antibody between VH and VL in addition to CH1 and CL.

A “bispecific antibody” is an antibody as described herein wherein one variable domain of the antibody binds to a first antigen whereas a second variable domain of the antibody binds to a second antigen, wherein said first and second antigens are not identical. The term “bispecific antibody” also encompasses biparatopic antibodies, wherein one variable domain of the antibody binds to a first epitope on an antigen whereas a second variable domain of the antibody binds a second epitope on the antigen. The term further includes antibodies wherein at least one VH is capable of specifically recognizing a first antigen and a VL, paired with the at least one VH in an immunoglobulin variable domain, is capable of specifically recognizing a second antigen. The resulting VH/VL pair will bind either antigen 1 or antigen 2, and are called “two-in-one antibodies”, described in for instance WO 2008/027236, WO 2010/108127 and Schaefer et al (Cancer Cell 20, 472-486, October 2011). A bispecific antibody according to the present invention is not limited to any particular bispecific format or method of producing it.

A multispecific antibody, such as a trispecific antibody as described herein, is an antibody wherein one variable domain of the antibody binds to a first antigen, a second variable domain of the antibody binds to a second antigen, and in case of a trispecific antibody a third variable domain of the antibody binds to a third antigen, wherein said first, second and third antigens are not identical or epitopes to which they bind are not identical. That is, a trispecific antibody may be triparatopic, in that it binds three different epitopes on the same antigen, or two epitopes on one antigen and one epitope on a second antigen.

Multivalent antibodies, such as bispecific or trispecific antibodies, have two or more binding domains. The binding domains may comprise a variable domain and a CH1/CL region. Some or all of the binding domains may be directed towards the same antigen however, typically, as is the case in the present invention at least two, and preferably at least three binding domains bind different antigens. In case of trispecific antibodies the three binding domains typically all bind a different antigen. Thus, the binding domains preferably all bind different antigens. In such case the binding domains all also have a different sequence.

Multivalent antibodies can be generated using various technologies, including cell fusion, chemical conjugation or recombinant DNA techniques. Multivalent antibody formats are known in the art. Examples are antibodies having two different binding domains, such as in bispecific antibodies, that may bind two different antigens, or two different epitopes within the same antigen. Such a format may allow for the use of calibrated binding that will allow the multivalent antibody to be selectively targeted to cells or targets that express two antigens or epitopes such as a tumor cell whilst not targeting healthy cells expressing one antigen, or to target such healthy cells expressing one antigen at lower expression levels. Similarly, having two different binding domains on a multivalent antibody, such as a bispecific antibody, may permit binding of different antigens, such that said multivalent antibody could be used to target both an inhibitory and a stimulatory molecule on a single cell or on two interacting cells to result in enhanced potency of the multivalent antibody. A multivalent antibody could also be used to redirect cells, for example immunomodulatory cells, that could be redirected to a tumor. Non-limiting examples of multivalent antibodies are described in the art. Multivalent antibodies are also described in WO 2019/190327 which is incorporated by reference herein.

In one aspect the invention provides a composition comprising a multivalent antibody comprising a first variable domain with VH1 that binds a first tumor antigen (TA1), a second variable domain with VH3 that binds a second tumor antigen (TA2), and a third variable domain with VH2 that binds an immune cell engaging antigen (IEA); and wherein the composition further comprises a second binding molecule that binds TA1 or TA2.

The variable domain of the multivalent antibody with VH2 that binds an immune cell engaging antigen (IEA) can bind to any molecule expressed on the surface of an immune effector cell, such as for example CD3, TCR-α chain, or TCR-β chain. Other suitable immune cell engaging antigens are for example, but not limited to, CD2, CD4, CD5, CD7, CD8, CD137, CD28, CD16, CD16A, CD64, OX40, CD27, CD40, ICOS, GITR, NKG2D, NKp46, NKp44, and NKp30. Preferably, this variable domain binds to CD3, TCR-α chain, TCR-β chain, CD2, or CD5. This variable domain preferably binds to CD3. Binding is preferably to an extracellular part of the immune cell engaging antigen (IEA). Preferably, the binding of the multivalent antibody to the IEA activates the immune effector cell or provides a costimulatory signal. Preferably, the binding of the multivalent antibody to the IEA activates the immune effector cell.

The term “CD3” (cluster of differentiation 3) refers a protein complex, which is composed of a CD3y chain (SwissProt P09693), a CD3δ chain (SwissProt P04234), CD3ε chains (SwissProt P07766), and a CD3 zeta chain homodimer (SwissProt P20963). CD3ε is known under various aliases some of which are: “CD3e Molecule, Epsilon (CD3-TCR Complex)”; “CD3e Antigen, Epsilon Polypeptide (TiT3 Complex)”; T-Cell Surface Antigen T3/Leu-4 Epsilon Chain; T3E; T-Cell Antigen Receptor Complex, Epsilon Subunit Of T3; CD3e Antigen; CD3-Epsilon 3; IMD18; TCRE. Ids for CD3E Gene are HGNC: 1674; Entrez Gene: 916; Ensembl: ENSG00000198851; OMIM: 186830 and UniProtKB: P07766. These chains associate with the T-cell receptor (TCR) and the ζ-chain to form a TCR complex that can upon mitogenic signaling generates an activation signal in T lymphocytes. CD3 is expressed on T cells and NK T cells. Where reference is made to CD3 herein, the reference is to human CD3, unless specifically stated otherwise.

The CD3 binding domain may range in affinity, epitope and other characteristics. Specific variable domains that can bind an extracellular part of CD3 are variable domains that comprise at least one heavy chain complementarity determining region (CDR) selected from the group consisting of SEQ ID NO: 2, SEQ ID NO:3, SEQ ID NO:4, SEQ ID NO:6, SEQ ID NO:7, SEQ ID NO:9, SEQ ID NO:10, SEQ ID NO:12, SEQ ID NO:13, SEQ ID NO:14, SEQ ID NO:16 SEQ ID NO:17 SEQ ID NO:18 SEQ ID NO: 20, SEQ ID NO:21, SEQ ID NO:23, SEQ ID NO:24, and SEQ ID NO:25.

The CD3 antigen binding domain can comprise the heavy chain CDR1 of SEQ ID NO: 2, SEQ ID NO: 6, SEQ ID NO:9, SEQ ID NO:12, SEQ ID NO:16, SEQ ID NO:20, or SEQ ID NO: 23, the heavy chain CDR2 of SEQ ID NO:3, SEQ ID NO: 7, SEQ ID NO:10, SEQ ID NO:13, SEQ ID NO:17, or SEQ ID NO:24, and the heavy chain CDR3 of SEQ ID NO: 4, SEQ ID NO:14, SEQ ID NO:18, SEQ ID NO:21, or SEQ ID NO:25.

The CD3 antigen binding domain can comprise a heavy chain CDR1, CDR2, and/or CDR3 sequence that is at least about 80, 85%, 90%, 95%, 96%, 97%, 98%, 99% or 100% identical to an amino acid sequence selected from the group consisting of: SEQ ID NO: 2, SEQ ID NO:3, SEQ ID NO:4, SEQ ID NO:6, SEQ ID NO:7, SEQ ID NO:9, SEQ ID NO:10, SEQ ID NO:12, SEQ ID NO:13, SEQ ID NO:14, SEQ ID NO:16 SEQ ID NO:17 SEQ ID NO:18 SEQ ID NO: 20, SEQ ID NO:21, SEQ ID NO:23, SEQ ID NO:24, and SEQ ID NO:25.

The CD3 antigen binding domain can comprise a heavy chain variable region sequence that is at least about 95%, 96%, 97%, 98%, 99% or 100% identical to an amino acid sequence selected from the group of: SEQ ID NO: 1, SEQ ID NO:5, SEQ ID NO:8, SEQ ID NO:11, SEQ ID NO:15, SEQ ID NO:19 and SEQ ID NO:22.

The CD3 binding domain can comprise a heavy chain variable region with the amino acid sequence of SEQ ID NO: 1, SEQ ID NO:5, SEQ ID NO:8, SEQ ID NO:11, SEQ ID NO:15, SEQ ID NO:19 and SEQ ID NO:22, and a light chain variable region comprising the amino acid sequence of SEQ ID NO: 93 or SEQ ID NO:99, with 0-10, preferably 0-5, amino acid insertions, deletions, substitutions, additions or a combination thereof.

The CD3 antigen binding domain can comprise the heavy chain variable region of SEQ ID NO: 1, SEQ ID NO:5, SEQ ID NO:8, SEQ ID NO:11, SEQ ID NO:15, SEQ ID NO:19 and SEQ ID NO:22 and a light chain variable region comprising the amino acid sequence of SEQ ID NO: 93 or SEQ ID NO:99.

In certain embodiments, the variable domain of the multivalent antibody with VH1 binds to TA1.

TA1 can be any antigen expressed on a tumor cell. TA1 is preferably PD-L1, PD-L2, HVEM, CD47, B7-H3, B7-H4, B7-H7, or Siglec-15.

TA1 is preferably a member of an immune checkpoint receptor/ligand pair, such as for instance PD-L1 or PD-L2. The variable domain inhibits the signaling pathway of the pair and thereby stimulates an immune response that would otherwise be suppressed to at least some extent.

PD-L1 is a type 1 transmembrane protein that plays a role in suppressing an immune response during particular events such as pregnancy, tissue allografts, autoimmune disease and other disease states such as hepatitis. The binding of PD-L1 to PD-1 or B7.1 (CD80) transmits an inhibitory signal which reduces the proliferation of the PD-1 expressing T cells. PD-1 is thought to be able to control the accumulation of foreign antigen specific T cells through apoptosis. PD-L1 is expressed by a variety of cancer cells and the expression thereof is thought to be at least in part responsible for a dampening of an immune response against the cancer cell. PD-L1 is a member of the B7-family of protein and is known under a variety of other names such as CD274 Molecule; CD274 Antigen; B7 Homolog 1; PDCD1 Ligand 1; PDCD1LG1; PDCD1L1; B7H1; PDL1; Programmed Cell Death 1 Ligand 1; Programmed Death Ligand 1; B7-H1; and B7-H. External Ids for CD274 are HGNC: 17635; Entrez Gene: 29126; Ensembl: ENSG00000120217; OMIM: 605402; UniProtKB: Q9NZQ7.

PD-L2 is a second ligand for PD-1. Engagement of PD-1 by PD-L2 inhibits T cell receptor (TCR)-mediated proliferation and cytokine production by CD4+ T cells. At low antigen concentrations, PD-L2/PD-1 binding inhibits B7-CD28 signals. At high antigen concentrations, PD-L2/PD-1 binding reduces cytokine production. PD-L expression is up-regulated on antigen-presenting cells by interferon gamma treatment. It is expressed in some normal tissues and a variety of tumors. PD-L1 and PD-L2 are thought to have overlapping functions and regulate T cell responses. The protein is known under a number of other names such as Programmed Cell Death 1 Ligand 2; B7 Dendritic Cell Molecule; Programmed Death Ligand 2; Butyrophilin B7-DC; PDCD1 Ligand 2; PD-1 Ligand 2; PDCD1L2; B7-DC; CD273; B7DC; PDL2; PD-1-Ligand 2; CD273 Antigen; BA574F11.2; and Btdc. External Ids for PD-L2 are HGNC: 18731; Entrez Gene: 80380; Ensembl: ENSG00000197646; OMIM: 605723; and UniProtKB: Q9BQ51.

HVEM, also known as tumor necrosis factor receptor superfamily member 14 (TNFRSF14) and CD270, is a human cell surface receptor of the TNF-receptor (tumor necrosis factor) superfamily. In humans, the protein is encoded by the TNFRSF14 gene. HVEM can engage at least four distinct ligands, the TNFSF members LIGHT (TNFSF14) and TNFB/LTa (tumor necrosis factor B/lymphotoxin a) and immunoglobulin superfamily members B- and T- lymphocyte attenuator (BTLA) and CD160. For a reference sequence of human HVEM, we refer to SwissProt no. Q92956.3; aa1-283. The reference is solely made to identify a HVEM gene/protein. It is not intended to limit the HVEM as described herein to the particular sequence of the database entry. Natural variants of HVEM that can bind BTLA, CD160, LIGHT and TNFB and can be bound by an antibody as described herein are within the scope of the invention.

CD47 is a transmembrane protein that in humans is encoded by the CD47 gene. The protein is known under a number of other names such as integrin associate protein (IAP), MER6, OA3 and CD47 molecule. CD47 belongs to the immunoglobulin superfamily and can bind the ligands thrombospondin-1 (TSP-1) and signal-regulatory protein alpha (SIRPa). CD47 is ubiquitously expressed in human cells and has been found to be overexpressed in many different tumor cells. There are four alternatively spliced isoforms of CD47. External IDs for CD47s are HGNC: 1682, OMIM: 601028, Entrez Gene: 961, Ensembl: ENSG00000196776 and UniProtKB: Q08722.

B7-H3, an immune checkpoint molecule, is a costimulatory B7 molecule that signals through CD28 family molecules such as CD28, CTLA-4 and ICOS. The protein is known under a number of other names such as Cluster of Differentiation 276 (CD276), 4Ig-B7-H3, B7H3, B7RP-2 and CD276 molecule. B7-H3 has been found to be overexpressed by solid tumors. External IDs for B7-H3 are HGNC: 19137, OMIM: 605717, Entrez Gene: 80381, Ensembl: ENSG00000103855 and UniProtKB: Q5ZPR3.

B7-H4, an immune checkpoint molecule, and belongs to the B7 family of costimulatory molecules. In humans, the protein is encoded by the VTCN1 gene. The protein is known under a number of other names such as V-set domain-containing T-cell activation inhibitor 1 (VTCN1), B7H4, B7S1, B7X, B7h0.5, PRO1291, VCTN1. External IDs for B7-H4 are HGNC: 28873, OMIM: 608162, Entrez Gene: 79679, Ensembl: ENSG00000134258 and UniProtKB: Q7Z7D3.

B7-H7, previously known as human endogenous retrovirus-H long terminal repeart associating 2 (HHLA2), belongs to the B7 family of costimulatory molecules. B7-H7 has been identified as a specific ligand for human CD28H, which together promote CD4+ T-cell proliferation and cytokine production. External IDs for B7-H7 are HGNC: 4905, Entrez Gene: 11148, Ensembl: ENSG00000114455, OMIM: 604371 and UniProtKB: Q9UM44.

Siglec-15, Sialic acid-binding immunoglobulin-type lectins, is a cell surface protein that binds sialic acid and is primarily found on the surface of immune cells. The protein is known under a number of other names such as CD33 Antigen-like 3, CD33 Molecule-like 3, CD33L3 and Sialic Acid Binding Ig Like Lectin 15. External IDs for Siglec-15 are HGNC: 27596, OMIM: 618105, Entrez Gene: 284266, Ensembl: ENSG00000197046 and UniProtKB: Q6ZMC9.

In certain embodiments the TA1 binding domain of the multivalent antibody specifically binds human PD-L1. The PD-L1 binding domain or variable domain of the multivalent antibody may range in affinity, epitope and other characteristics. Specific variable domains that can bind an extracellular part of PD-L1 are variable domains that comprise at least one heavy chain CDR selected from the group consisting of SEQ ID NO: 27, SEQ ID NO:28, SEQ ID NO:29, SEQ ID NO: 31, SEQ ID NO:32, SEQ ID NO:33, SEQ ID NO: 35, SEQ ID NO:36, and SEQ ID NO:37, SEQ ID NO:39, SEQ ID NO: 40, SEQ ID NO: 41, SEQ ID NO: 43, SEQ ID NO: 44, and SEQ ID NO: 45.

The PD-L1 antigen binding domain can comprise the heavy chain CDR1 of SEQ ID NO: 27, SEQ ID NO:31, SEQ ID NO:35, SEQ ID NO: 39, or SEQ ID NO: 43, the heavy chain CDR2 of SEQ ID NO: 28, SEQ ID NO:32, SEQ ID NO:36, SEQ ID NO: 40, or SEQ ID NO: 44, and the heavy chain CDR3 of SEQ ID NO: 29, SEQ ID NO:33, SEQ ID NO:37, SEQ ID NO: 41, or SEQ ID NO: 45.

The PD-L1 antigen binding domain can comprise a heavy chain CDR1, CDR2 and/or CDR3 sequence that is at least about 80%, 85%, 90%, 95%, 96%, 97%, 98%, 99% or 100% identical to an amino acid sequence selected from the group consisting of: SEQ ID NO: 27, SEQ ID NO:28, SEQ ID NO:29, SEQ ID NO: 31, SEQ ID NO:32, SEQ ID NO:33, SEQ ID NO: 35, SEQ ID NO:36, SEQ ID NO:37, SEQ ID NO:39, SEQ ID NO: 40, SEQ ID NO: 41, SEQ ID NO: 43, SEQ ID NO: 44, and SEQ ID NO: 45.

The PD-L1 antigen binding domain can comprise a heavy chain variable region sequence that is at least about 95%, 96%, 97%, 98%, 99% or 100% identical to an amino acid sequence selected from the group of: SEQ ID NO: 26, SEQ ID NO:30, SEQ ID NO:34, SEQ ID NO: 38 and SEQ ID NO: 42.

The PD-L1 antigen binding domain can comprise a heavy chain variable region with the amino acid sequence of SEQ ID NO: 26, SEQ ID NO:30, SEQ ID NO:34, , SEQ ID NO: 38 and SEQ ID NO: 42, with 0-10, preferably 0-5 amino acid insertions, deletions, substitutions, additions or a combination thereof.

The PD-L1 antigen binding domain can comprise the heavy chain variable region of SEQ ID NO: 26, SEQ ID NO:30, SEQ ID NO:34, SEQ ID NO: 38 or SEQ ID NO: 42 and a light chain variable region comprising the amino acid sequence of SEQ ID NO: 93 or SEQ ID NO:99.

In certain embodiments, the PD-L1 antigen binding domain comprises the heavy and/or light chain, in particular heavy chain, variable regions of the PD-L1 antibodies comprising heavy chains having the amino acid sequences of SEQ ID NO: 46, SEQ ID NO: 47, SEQ ID NO: 51, or the amino acid sequences disclosed for MSB-0010718C, see WO 2013/079174; STI-1014 see WO2013/181634; CX-072, see WO2016/149201; KN035, see Zhang et al., Cell Discov. 7:3 (March 2017); LY3300054, see, e.g., WO 2017/034916; and CK-301, see Gorelik et al., AACR:Abstract 4606 (April 2016)), and 12A4 or MDX-1105, see, e.g., WO 2013/173223.

In certain embodiments, the PD-L1 antigen binding domain binds the same epitope as the heavy and light chain variable regions of the PD-L1 antibodies comprising heavy chains having SEQ ID NO: 46, SEQ ID NO: 47, SEQ ID NO: 51, or of MSB-0010718C, see WO 2013/079174; STI-1014 see WO2013/181634; CX-072, see WO2016/149201; KN035, see Zhang et al., Cell Discov. 7:3 (March 2017); LY3300054, see, e.g., WO 2017/034916; and CK-301, see Gorelik et al., AACR:Abstract 4606 (April 2016)), and 12A4 or MDX-1105, see, e.g., WO 2013/173223.

In certain embodiments, the PD-L1 antigen binding domain competes for binding to PD-L1 with the heavy and light chain variable regions of the PD-L1 antibodies MPDL3280A, RG7446, see US 2010/0203056 A1; MEDI-4736, see WO 2011/066389; MSB-0010718C, see WO 2013/079174; STI-1014 see WO2013/181634; CX-072, see WO2016/149201; KN035, see Zhang et al., Cell Discov. 7:3 (March 2017); LY3300054, see, e.g., WO 2017/034916; and CK-301, see Gorelik et al., AACR:Abstract 4606 (April 2016)), and 12A4 or MDX-1105, see, e.g., WO 2013/173223.

In certain embodiments, the variable domain of the multivalent antibody with VH3 binds to TA2.

TA2 can be any tumor associated antigen but is preferably CLEC12A or a member of ErbB family of proteins, preferably EGFR.

CLEC12A is also referred to as C-Type Lectin Domain Family 12, Member A; C-Type Lectin Protein CLL-1; MICL; Dendritic Cell-Associated Lectin 2; C-Type Lectin Superfamily; Myeloid Inhibitory C-Type Lectin-Like Receptor; C-Type Lectin-Like Molecule-1; CLL-1; DCAL2; CLL1; C-Type Lectin-Like Molecule 1; DCAL-2; Killer cell lectin like receptor subfamily L, member 1 (KLRL1); CD371 (Bakker A. et al. Cancer Res. 2004, 64, p8843 50; GenBankTM access.no: AY547296; Zhang W. et al. GenBankTM access.no: AF247788; A.S. Marshall, et al. J Biol Chem 2004, 279, p14792-802; GenBankTM access.no: AY498550; Y.Han et al. Blood 2004, 104, p2858 66; H.Floyd, et al. GenBankTM access.no: AY426759; C.H.Chen, et al. Blood 2006, 107, p1459 67). Ids: HGNC: 31713; Entrez Gene: 160364; Ensembl: ENSG00000172322; OMIM: 612088; UniProtKB: Q5QGZ9. CLEC12A is an antigen that is expressed on leukemic blast cells and on leukemic stem cells in acute myeloid leukemia (AML), including the CD34 negative or CD34 low expressing leukemic stem cells (side population) (A.B. Bakker et al. Cancer Res 2004, 64, p8443 50; Van Rhenen et al. 2007 Blood 110:2659; Moshaver et al. 2008 Stem Cells 26:3059). Expression of CLEC12A is otherwise thought to be restricted to the hematopoietic lineage, particularly to myeloid cells in peripheral blood and bone marrow, i.e., granulocytes, monocytes and dendritic cell precursors. More importantly, CLEC12A is absent on hematopoietic stem cells. This expression profile makes CLEC12A a particularly favorable target in AML. The full length form of CLEC12A comprises 275 amino acid residues, including an additional intracellular stretch of 10 amino acids which is absent in most other isoforms, and shows the strictly myeloid expression profile (surface expression and mRNA level). The term ‘CLEC12A or functional equivalent thereof means all (such as splice and mutation) variants that are referenced above and isoforms thereof that retain the strict myeloid expression profile (both at surface expression level and mRNA level) as described in Bakker et al. Cancer Res 2004, 64, p8443-50 and Marshall 2004 - J Biol Chem 279(15), p14792-802. A CLEC12A binding antibody of the invention binds human CLEC12A. Where herein reference is made to CLEC12A, the reference is to human CLEC12A, unless specifically stated otherwise.

‘ErbB1’ or ‘EGFR’ is a member of a family of four receptor tyrosine kinases (RTKs), named Her- or cErbB-1, -2, -3 and -4. The EGFR has an extracellular domain (ECD) that is composed of four sub-domains, two of which are involved in ligand binding and one of which is involved in homo-dimerization and heterodimerization. The reference numbers used in this section refer to the numbering of the references in the list headed “References cited in the specification”. EGFR integrates extracellular signals from a variety of ligands to yield diverse intracellular responses. The major signal transduction pathway activated by EGFR is composed of the Ras-mitogen-activated protein kinase (MAPK) mitogenic signaling cascade. Activation of this pathway is initiated by the recruitment of Grb2 to tyrosine phosphorylated EGFR. This leads to activation of Ras through the Grb2-bound Ras-guanine nucleotide exchange factor Son of Sevenless (SOS). In addition, the PI3-kinase-Akt signal transduction pathway is also activated by EGFR, although this activation is much stronger in case there is co-expression of Her3. The EGFR is implicated in several human epithelial malignancies, notably cancers of the breast, bladder, non-small cell lung cancer lung, colon, ovarian head and neck and brain. Activating mutations in the gene have been found, as well as over-expression of the receptor and of its ligands, giving rise to autocrine activation loops. This RTK has therefore been extensively used as target for cancer therapy. Both small-molecule inhibitors targeting the RTK and monoclonal antibodies (mAbs) directed to the extracellular ligand-binding domains have been developed and have shown hitherto several clinical successes, albeit mostly for a select group of patients. A database accession number for the human EGFR protein and the gene encoding it is (GenBank NM_005228.3). The accession number is primarily given to provide a further method of identification of EGFR protein as a target, the actual sequence of the EGFR protein bound by an antibody may vary, for instance because of a mutation in the encoding gene such as those occurring in some cancers or the like. Where reference herein is made to EGFR, the reference refers to human EGFR unless otherwise stated. The antigen-binding site that binds EGFR, binds EGFR and a variety of variants thereof such as those expressed on some EGFR positive tumors.

‘ErbB-2’ or ‘HER2’ as used herein refers to the protein that in humans is encoded by the ERBB-2 gene. Alternative names for the gene or protein include CD340; HER-2; HER-2/neu; MLN 19; NEU; NGL; TKR1. The ERBB-2 gene is frequently called HER2 (from human epidermal growth factor receptor 2). Where reference is made herein to ErbB-2, the reference refers to human ErbB-2. An antibody comprising an antigen-binding site that binds ErbB-2, binds human ErbB-2. The ErbB-2 antigen-binding site may, due to sequence and tertiary structure similarity between human and other mammalian orthologs, also bind such an ortholog but not necessarily so. Database accession numbers for the human ErbB-2 protein and the gene encoding it are (NP_001005862.1, NP_004439.2 NC_000017.10 NT_010783.15 NC_018928.2). The accession numbers are primarily given to provide a further method of identification of ErbB-2 as a target, the actual sequence of the ErbB-2 protein bound the antibody may vary, for instance because of a mutation in the encoding gene such as those occurring in some cancers or the like. The ErbB-2 antigen binding site binds ErbB-2 and a variety of variants thereof, such as those expressed by some ErbB-2 positive tumor cells.

‘ErbB-3’ or ‘HER3’ as used herein refers to the protein that in humans is encoded by the ERBB-3 gene. Alternative names for the gene or protein are HER3; LCCS2; MDA-BF-1; c-ErbB-3; c-erbb-3; erbb-3-S; p180-Erbb-3; p45-sErbb-3; and p85-sErbb-3. Where reference is made herein to ErbB-3, the reference refers to human ErbB-3. An antibody comprising an antigen-binding site that binds ErbB-3, binds human ErbB-3. The ErbB-3 antigen-binding site, may, due to sequence and tertiary structure similarity between human and other mammalian orthologs, also bind such an ortholog but not necessarily so. Database accession numbers for the human ErbB-3 protein and the gene encoding it are (NP_001005915.1 NP_001973.2, NC_000012.11 NC_018923.2 NT_029419.12). The accession numbers are primarily given to provide a further method of identification of ErbB-3 as a target, the actual sequence of the ErbB-3 protein bound by an antibody may vary, for instance because of a mutation in the encoding gene such as those occurring in some cancers or the like. The ErbB-3 antigen binding site binds ErbB- 3 and a variety of variants thereof, such as those expressed by some ErbB-2 positive tumor cells.

In certain embodiments the target cell antigen binding specifically binds human epidermal growth factor receptor (EGFR). The EGFR binding domain may range in affinity, epitope and other characteristics. Specific variable domains that can bind an extracellular part of EGFR are variable domains that comprise at least one heavy chain CDR selected from the group consisting of SEQ ID NO: 53, SEQ ID NO:54, SEQ ID NO:55, SEQ ID NO:57, SEQ ID NO:59, SEQ ID NO: 61 and SEQ ID NO:63.

The EGFR antigen binding domain can comprise the heavy chain CDR1 of SEQ ID NO: 53, the heavy chain CDR2 of SEQ ID NO: 54, and the heavy chain CDR3 of SEQ ID NO: 55, SEQ ID NO:57, SEQ ID NO:59, SEQ ID NO:61 or SEQ ID NO:63.

The EGFR antigen binding domain can comprise a heavy chain CDR1, CDR2 and/or CDR3 sequence that is at least about 80%, 85%, 90%, 95%, 96%, 97%, 98%, 99% or 100% identical to an amino acid sequence selected from the group consisting of: SEQ ID NO: 53, SEQ ID NO: 54, SEQ ID NO: 55, SEQ ID NO:57, SEQ ID NO:59, SEQ ID NO:61 and SEQ ID NO:63.

The EGFR antigen binding domain can comprise a heavy chain variable region sequence that is at least about 95%, 96%, 97%, 98%, 99% or 100% identical to an amino acid sequence selected from the group of: SEQ ID NO: 52, SEQ ID NO:56, SEQ ID NO:58, SEQ ID NO:60 and SEQ ID NO:62.

The EGFR binding domain can comprise a heavy chain variable region with the amino acid sequence of SEQ ID NO: 52, SEQ ID NO:56, SEQ ID NO:58, SEQ ID NO:60 and SEQ ID NO:62, with 0-10, preferably 0-5, amino acid insertions, deletions, substitutions, additions or a combination thereof.

The EGFR antigen binding domain can comprise the heavy chain variable region of SEQ ID NO: 52, SEQ ID NO:56, SEQ ID NO:58, SEQ ID NO:60 or SEQ ID NO:62 and a light chain variable region comprising the amino acid sequence of SEQ ID NO: 93 or SEQ ID NO:99.

In certain embodiments, the EGFR antigen binding domain comprises the heavy and/or light chain variable regions of the EGFR antibodies cetuximab or panitumumab.

In certain embodiments, the EGFR antigen binding domain binds the same epitope as the heavy and light chain variable regions of the EGFR antibodies cetuximab or panitumumab.

In certain embodiments, the EGFR antigen binding domain competes for binding to EGFR with the heavy and light chain variable regions of the EGFR antibodies cetuximab or panitumumab.

In certain embodiments the target cell antigen binding specifically binds human CLEC12A. The CLEC12A binding domain may range in affinity, epitope and other characteristics. Specific variable domains that can bind an extracellular part of CLEC12A are variable domains that comprise at least one heavy chain CDR selected from the group consisting of SEQ ID NO: 65, SEQ ID NO: 66, and SEQ ID NO: 67.

The CLEC12A antigen binding domain can comprise the heavy chain CDR1, CDR2, and CDR3 of SEQ ID NO:65, SEQ ID NO: 66, and SEQ ID NO: 67, respectively.

The CLEC12A antigen binding domain can comprise a heavy chain CDR1, CDR2 and/or CDR3 sequence that is at least about 80%, 85%, 90%, 95%, 96%, 97%, 98%, 99% or 100% identical to the amino acid sequence of SEQ ID NO:65, SEQ ID NO: 66, or SEQ ID NO: 67.

The CLEC12A antigen binding domain can comprise a heavy chain variable region sequence that is at least about 95%, 96%, 97%, 98%, 99% or 100% identical to the amino acid sequence of SEQ ID NO: 64.

The CLEC12A binding domain can comprise a heavy chain variable region with the amino acid sequence of SEQ ID NO: 64, with 0-10, preferably 0-5, amino acid insertions, deletions, substitutions, additions or a combination thereof.

The CLEC12A antigen binding domain can comprise the heavy chain variable region of SEQ ID NO: 64, and a light chain variable region comprising the amino acid sequence of SEQ ID NO: 93 or SEQ ID NO:99.

In one embodiment, the multivalent antibody of the invention comprises a first variable domain with VH1 that binds PD-L1, a second variable domain with VH2 that binds CD3, and a third variable domain with VH3 that binds EGFR, wherein the variable domains are as defined herein. The second binding molecule can be any binding molecule with specificity for TA1 or TA2, preferably, TA1. TA1 is preferably PD-L1. Such binding molecule includes, but is not limited to, an antibody, or fragment or variant thereof that maintains the binding specificity of said antibody, or a structure comprising said fragment.

Combining the multivalent antibody with a second binding molecule permits the multivalent antibody to only, or mainly, induce cell killing of cells expressing both antigens, TA1 and TA2 (e.g. PD-L1 and EGFR), such as tumor cells. The multivalent antibody should not induce cell killing of cells expressing only TA1 or TA2 (e.g. PD-L1 but not EGFR; or EGFR but not PD-L1), such as non-tumor cells, or at least to a lesser extent than in the absence of the second binding molecule.

The combination of a multivalent antibody with a second binding molecule is particularly useful in a situation where there are non-tumor cells that express TA1 but not TA2, and non-tumor cells that express TA2 but not TA1, and the avidity of the multivalent antibody is insufficient to only, or mainly, induce cell killing of cells expressing both TA1 and TA2. If the multivalent antibody still binds to and/or induces cell killing of non-tumor cells expressing TA1 but not TA2, the second binding molecule as described herein binds to TA1. This prevents or reduces the binding of a multivalent antibody as described herein to the non-tumor cells expressing TA1 but not TA2, and/or reduces cell killing of the non-tumor cells induced by the multivalent antibody. Likewise, if the multivalent antibody still binds to and/or induces cell killing of non-tumor cells expressing TA2 but not TA1, the second binding molecule as described herein binds to TA2. This prevents or reduces the binding of a multivalent antibody as described herein to the non-tumor cells expressing TA2 but not TA1, and/or for reduces cell killing of the non-tumor cells induced by the multivalent antibody.

The second binding molecule of the invention binding to TA1 or TA2 competes with the multivalent antibody for binding to TA1 or TA2. The selective activity against dual positive TA1, TA2 expressing cells may be caused by superior binding of the multivalent antibody to these cells due to affinity of the TA1 or TA2 binding domains of the multivalent antibody and the second binding molecule, the valency of the second binding molecule, epitope specificities of the multivalent antibody and second binding molecule, internalization or shedding of the target antigen due to the second binding molecule, or a combination of these aspects. As such, the second binding molecule mitigates the multivalent antibody’s binding to TA1 or TA2, or causes reduced binding of the multivalent antibody to TA1 cells that lack or have reduced expression of TA2, or TA2 cells that lack or have reduced expression of TA1.

The affinity of the TA1 and TA2 binding domains of the multivalent antibody may be chosen based on the expression levels of TA1 and TA2 on tumor cells and non-tumor cells. For instance, if the expression level of TA2 on tumor cells is higher than that of TA1, the affinity of the TA2 binding domain of the multivalent antibody may be low or low-medium affinity, such as for instance double or triple digit nM, and the TA1 binding domain of the multivalent antibody medium or medium-high affinity, such as for instance single or double digit nM. Likewise, if the expression level of TA1 on tumor cells is higher than that of TA2, the affinity of the TA1 binding domain of the multivalent antibody may be low or low-medium affinity, such as for instance double or triple digit nM, and the TA2 binding domain of the multivalent antibody medium or medium-high affinity, such as for instance single or double digit nM. If the expression level of TA2 on tumor cells is comparable to that of TA1, the affinity of the TA2 binding domain and of the TA1 binding domain of the multivalent antibody are preferably in the same range, such as for instance in the high, medium-high, medium, low-medium, or low affinity range. If the expression level of TA2 on tumor cells is lower than that of TA1, the affinity of the TA2 binding domain of the multivalent antibody may be medium-high or high affinity, and the TA1 binding domain of the multivalent antibody low, low-medium, or medium affinity. Likewise, if the expression level of TA1 on tumor cells is lower than that of TA2. The affinity of the TA1 binding domain of the multivalent antibody may then be medium-high or high affinity, and the TA2 binding domain of the multivalent antibody low, low-medium, or medium affinity.

The second binding molecule is preferably a full length antibody, a Fab, a modified Fab, or a scFv. The second binding molecule preferably does not comprise a TA2 binding variable domain. It preferably does not comprise a binding domain that binds an immune cell engaging antigen (IEA). The TA1 or TA2 binding variable domain of the multivalent antibody may be the same as a TA1 or TA2 binding variable domain of the second binding molecule. The second binding molecule comprises at least one TA1 or TA2 binding variable domain but may also comprise multiple TA1 or TA2 binding variable domains. The second binding molecule preferably comprises two TA1 or TA2 binding variable domains. The TA1 or TA2 binding variable domains of the second binding molecule are conveniently but not necessarily the same. The second binding molecule is preferably a bivalent monospecific antibody comprising two identical TA1 or TA2 binding variable domains. In certain embodiments, the second binding molecule has a lower avidity than the multivalent antibody, as measured in the same assay. An example of a suitable assay is a FACS binding assay.

The second binding molecule can be a commercially available antibody such as for instance atezolizumab or durvalumab, or an analogue or variant thereof. Another anti-PD-L1 antibody that may be used is that comprising heavy chains having SEQ ID NO: 47, or a functionally equivalent thereof. Further examples include, but are not limited to, MSB-0010718C, see WO 2013/079174; STI-1014 see WO2013/181634; CX-072, see WO2016/149201; KN035, see Zhang et al., Cell Discov. 7:3 (March 2017); LY3300054, see, e.g., WO 2017/034916; and CK-301, see Gorelik et al., AACR:Abstract 4606 (April 2016)), and 12A4 (also known as MDX-1105), see, e.g., WO 2013/173223.

In one embodiment, the second binding molecule comprises two heavy chains having SEQ ID NO: 46, 47 or 51.

The multivalent antibody should be competing with the second binding molecule for binding to TA1 or TA2. The multivalent antibody should be capable of outcompeting the second binding molecule for binding to cells expressing both TA1 and TA2, and the second binding molecule capable of out competing the multivalent molecule for binding cells that express only one of the antigens (TA1 or TA2) targeted by the second binding molecule.

Obtaining the correct targeting—e.g., the second binding molecule binding a single antigen expressing cell and the multivalent antibody binding the dual antigen cell in greater ratios may be achieved by the invention set out herein.

This may be achieved by modulating the affinity of the TA1 or TA2 binding domain of the multivalent antibody and/or the TA1 or TA2 binding domain of the second binding molecule. The modulation of the affinity of the TA1 or TA2 binding domain of the multivalent antibody and/or the TA1 or TA2 binding domain of the second binding molecule may be based on the expression levels of TA1 and TA2 on tumor cells and non-tumor cells. It is preferred that the k_{d} of the second binding molecule that binds TA1 or TA2 is comparable to, equal to, or lower than the k_{d} of the TA1 or TA2 binding domain of the multivalent antibody. The k_{d} is determined by the kₒₙ rate and the k_{off} rate. It may be preferred that the kₒₙ rate of the second binding molecule that binds TA1 or TA2 is comparable to, equal to, or higher than the kₒₙ rate of the TA1 or TA2 binding domain of the multivalent antibody. It may also be preferred that the k_{off} rate of the second binding molecule that binds TA1 is comparable to, equal to, or lower than the k_{off} rate of the TA1 or TA2 binding domain of the multivalent antibody. It may also be preferred that the kₒₙ rate of the second binding molecule that binds TA1 or TA2 is comparable to, equal to, or higher than the kₒₙ rate of the TA1 or TA2 binding domain of the multivalent antibody and that the k_{off} rate of the second binding molecule that binds TA1 is comparable to, equal to, or lower than the k_{off} rate of the TA1 or TA2 binding domain of the multivalent antibody. This allows for the second binding molecule to bind more strongly to TA1 or TA2, and/or occupy more TA1 or TA2, than the multivalent antibody, thereby preventing the multivalent antibody from binding to TA1 or TA2. When a cell expresses both TA1 and TA2, the multivalent antibody will bind the tumor-associated antigen (TA1 or TA2) not bound by the second binding molecule and outcompete the second binding molecule for binding to the tumor-associated antigen bound to it due to a greater avidity.

This may be enhanced by selecting TA1 and TA2 in such a way that the tumor-associated antigen not bound by the second binding molecule is present in excess over the tumor-associated antigen targeted by the second binding molecule, and/or by choosing a binding arm of the multivalent antibody that has high affinity for the tumor-associated antigen not targeted by the second binding molecule, or a combination of both. These modes of action, exemplified herein but not limited thereto, mitigate the multivalent antibody for binding to non-tumor cells that only express TA1 or TA2, or do so to a lesser extent than target tumor cells that express both TA1 and TA2.

In addition to affinity and avidity driving selectivity for the multivalent targeting of dual antigen expressing cells, other mechanistic means may be employed. It may be preferred that the second binding molecule causes internalization or shedding of the targeted antigen (TA1 or TA2). An array of tumor associated antigens are known in the art that have the capacity to internalize or shed upon being bound. This feature of the second binding molecule removes the antigen target for the multivalent molecule for a single expressing cells, whereas for the dual expressing cells, the multivalent will dock on a second antigen, not targeted by the binding molecule, and then lock onto the antigen targeted by the second binding molecule that reemerges over time.

Similarly, the multivalent molecule can be designed to have a targeting domain, which alters the antigen upon binding rendering the second binding molecule incapable of targeting the antigen. In any event, the targeting of one molecule (multivalent molecule or the second binding molecule) should disrupt the potential of the second molecule to target the same antigen already bound by the first.

In one aspect the affinity of the TA1 or TA2 binding variable domain of the second binding molecule is comparable to the affinity of the TA1 or TA2 binding variable domain of the multivalent antibody. This allows for the multivalent antibody to outcompete the second binding molecule or have enhanced binding to TA1 or TA2 on cells expressing both TA1 and TA2. To increase the binding of the second binding molecule to TA1 or TA2 on cells expressing only one of TA1 and TA2, the valency and/or affinity of the second binding molecule may be increased.

In one aspect the affinity of the TA1 or TA2 binding variable domain of the second binding molecule is equal to the affinity of the TA1 or TA2 binding variable domain of the multivalent antibody. This allows for the multivalent antibody to outcompete the second binding molecule or have enhanced binding to TA1 or TA2 on cells expressing both TA1 and TA2. To increase the binding of the second binding molecule to TA1 or TA2 on cells expressing only one of TA1 and TA2, the valency and/or affinity of the second binding molecule may be increased.

In one aspect the affinity of the TA1 or TA2 binding variable domain of the second binding molecule is higher than the affinity of the TA1 or TA2 binding variable domain of the multivalent antibody. This allows for the second binding molecule to outcompete the multivalent antibody for binding to TA1 or TA2. To increase the binding of the multivalent antibody to TA1 on cells expressing both TA1 and TA2, and thus outcompete the second binding molecule for binding to TA1, the affinity of the TA2 binding variable domain of the multivalent antibody may be increased.

The k_{d} or kₒₙ or k_{off} of a variable domain as described herein is preferably measured in a biacore and preferably in a bispecific monovalent format, i.e. using a bispecific antibody that has one variable domain of which the k_{d} or kₒₙ or k_{off} is to be determined and one variable domain that binds an irrelevant target. In the present application this irrelevant target suitably is a tetanus toxoid binding domain, preferably having the same common light chain and the VH chain of SEQ ID NO: 68.

The additional variable domain of the multivalent antibody that binds TA1 is preferably present as part of an scFv domain, a Fab domain or a modified Fab domain. Preferably the additional variable is associated with a CH1 region at its C-terminus and which is linked, preferably by means of a linker, to the N-terminus of the variable domain that binds the immune cell engaging antigen (IEA). Preferably, the additional binding domain is a Fab domain comprising a heavy chain variable region (VH) and a light chain variable region (VL), said heavy chain variable region of said Fab domain comprising a CH1 region (VH-CH1) and said light chain variable region of said Fab comprising a CL region (VL-CL). The additional binding domain can also be a modified Fab domain consisting of a VH-CH1 and VL. Alternatively, the additional binding domain is a modified Fab domain consisting of a VL-CL and a VH. In such modified Fab domains, a constant region, CH1 or CL, is present which is not paired with its cognate region and/or a variable region VH or VL, is present, which is not paired with its cognate region.

The variable domain of the multivalent antibody that binds an immune cell engaging antigen (IEA) and/or the variable domain of the multivalent antibody that binds TA2 are preferably also associated with a CH1 region. Preferably, the binding domain that binds an immune cell engaging antigen (IEA) and/or the binding domain that binds TA2 is a Fab domain comprising a heavy chain variable region (VH) and a light chain variable region (VL), said heavy chain variable region of said Fab domain comprising a CH1 region (VH-CH1) and said light chain variable region of said Fab comprising a CL region (VL-CL). The binding domain that binds an immune cell engaging antigen (IEA) and/or the binding domain that binds TA2 can also be a modified Fab domain consisting of a VH-CH1 and VL. Alternatively, the additional binding domain is a modified Fab domain consisting of a VL-CL and a VH. In such modified Fab domains, a constant region, CH1 or CL, is present which is not paired with its cognate region and/or a variable region VH or VL, is present, which is not paired with its cognate region.

A linker may be used to connect the additional binding domain to the base antibody. The linker can be any suitable linker known in the art, and preferably comprises a peptide region, for example one or more hinge regions and/or one or more regions derived from a hinge region. The combination of the linker and a constant region (e.g., CH1) to which it is connected may determine properties of the multivalent antibody. A linker may allow correct functionality of the antibody and/or orientation of the one or more additional binding domains to the base antibody. The combination of a CH1 region in the binding domain may improve functionality of the antibody and/or orientation of the binding domains to the base antibody. A linker sequence that is based on a hinge of a given subtype is preferably combined with a constant region of the same subtype in the additional binding domain.

Preferably, the linker is a naturally occurring sequence, or based on a naturally occurring sequence. More specifically, said linker is preferably a hinge sequence or comprises a sequence based on a hinge sequence. More specifically said linker may comprise a hinge region based on an IgG1 hinge region, an IgG2 hinge region, an IgG3 hinge region or an IgG4 hinge region. The linker is preferably a peptide of 7-30 amino acid residues. The linker preferably comprises a hinge sequence of an antibody as described herein.

Alternatively, said linker comprises a peptide of 7-30 amino acid residues comprising one or more of the following sequences:

1: ESKYGPP (SEQ ID NO: 69);
2: EPKSCDKTHT (SEQ ID NO: 70);
3: GGGGSGGGGS (SEQ ID NO: 71);
4: ERKSSVESPPSP (SEQ ID NO: 72);
5: ERKCSVESPPSP (SEQ ID NO: 73);
6: ELKTPLGDTTHT (SEQ ID NO: 74);
7: ESKYGPPSPSSP (SEQ ID NO: 75);
8: ERKSSVEAPPVAG (SEQ ID NO: 76);
9: ERKCSVEAPPVAG (SEQ ID NO: 77);
10: ESKYGPPAPEFLGG (SEQ ID NO: 78);
11: EPKSCDKTHTSPPSP (SEQ ID NO: 79);
12: EPKSCDGGGGSGGGGS (SEQ ID NO: 80);
13: GGGGSGGGGSAPPVAG (SEQ ID NO: 81);
14: EPKSCDKTHTAPELLGG (SEQ ID NO: 82);
15: ERKSSVESPPSPAPPVAG (SEQ ID NO: 83);
16: ERKCSVESPPSPAPPVAG (SEQ ID NO: 84);
17: ELKTPLGDTTHTAPEFLGG (SEQ ID NO: 85);
18: ESKYGPPSPSSPAPEFLGG (SEQ ID NO: 86);
19: EPKSCDKTHTSPPSPAPELLGG (SEQ ID NO: 87);
20: ERKSSVEEAAAKEAAAKAPPVAG (SEQ ID NO: 88);
21: ERKCSVEEAAAKEAAAKAPPVAG (SEQ ID NO: 89);
22: ESKYGPPEAAAKEAAAKAPEFLGG (SEQ ID NO: 90);
23: EPKSCDKTHTEAAAKEAAAKAPELLGG (SEQ ID NO: 91);
24: ELKTPLGDTTHTEAAAKEAAAKAPEFLGG (SEQ ID NO: 92);

or a sequence having at least about 85% sequence identity to any one thereof.

The linker that connects the base antibody to one or more additional binding domains is preferably a peptide comprising an amino acid sequence of any one of the peptide sequences 1 to 24 or a polypeptide comprising an amino acid sequence having at least about 85% sequence identity to the peptide sequences 1 to 24.

The binding domains of the multivalent antibody can have any suitable light chain. They can each have a different light chain or two or more binding domains can have the same or a similar light chain. Such light chain is referred to herein as a common light chain, which is a light chain comprising a common light chain variable region. The light chain constant region (CL) is not necessarily the same or similar in such common light chain. Preferably all binding domains of the multivalent antibody comprise a common light chain. The second binding molecule can also comprise a common light chain. Typically, this is the same common light chain as used in the multivalent antibody.

Having a common light chain or light chain variable region facilitates production of a multivalent antibody as it limits the number of different molecules that can be formed upon association of the immunoglobulin chains. A producing cell now needs only produce two heavy chains and one light chain or one light chain variable region. Where said light chain is expressed within a host cell that includes DNA encoding two heavy chains with three or more heavy chain variable regions, said light chain is capable of pairing with each available heavy chain variable region or CH1-VH1 region, thereby forming at least three functional antigen binding domains.

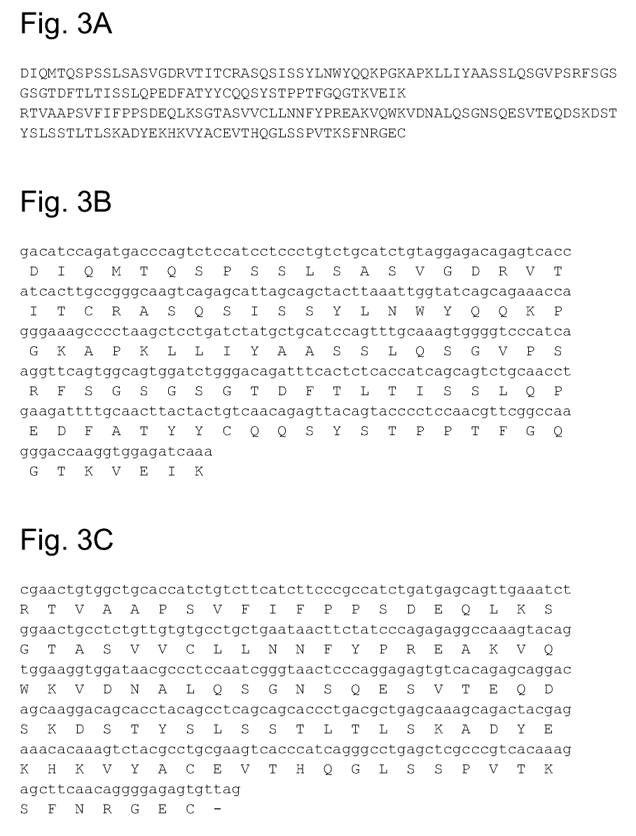

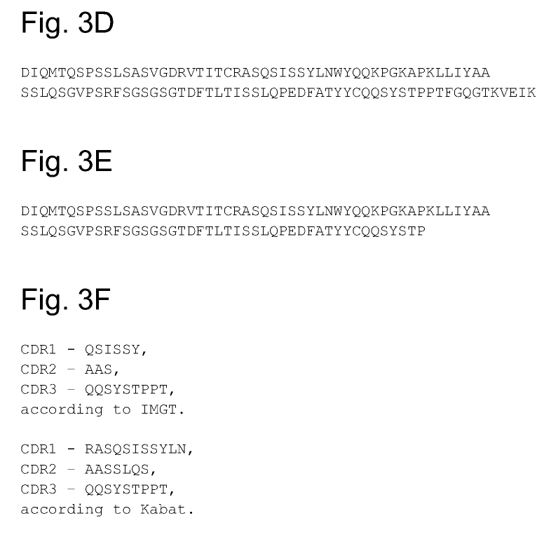

A common light chain or common light chain variable region is capable of pairing with different heavy chains or heavy chain variable regions, such as the heavy chains with VH1, VH2, and/or VH3. An example of such common light chain or common light chain variable region is described in WO2004/009618 and WO2009/157771. The common light chain or common light chain variable region preferably has a germline sequence. A preferred germline sequence is a light chain variable region that is frequently used in the human repertoire and has good thermodynamic stability, yield and solubility. A preferred germline light chain comprises a IgV_{K}1-39 variable region V-segment. A common light chain comprises preferably the rearranged germline human kappa light chain variable region IgV_{K}1-39*01/IGJ_{K}1*01 (FIG. 3B). It may comprise 0-5 amino acid insertions, deletions, substitutions, additions, or a combination thereof. The common light chain preferably further comprises a light chain constant region. This can be kappa or a lambda light chain constant region, preferably a kappa light chain constant region (FIG. 3C).

Preferably, a multivalent antibody of the invention comprises kappa light chain variable region IgV_{K}1-39*01/IGJ_{K}1*01 or IgV_{K}1-39*01/IGJ_{K}5*01. Preferably, the common light chain variable region in the multivalent antibody is IgV_{K}1-39*01/IGJ_{K}1*01 (SEQ ID NO: 93).

Preferably, a second binding molecule of the invention also comprises kappa light chain variable region IgV_{K}1-39*01/IGJ_{K}1*01 or IgV_{K}1-39*01/IGJ_{K}5*01. Preferably, the common light chain variable region in the second binding molecule is IgV_{K}1-39*01/IGJ_{K}1*01 (SEQ ID NO: 93).

Other common light chain variable regions are known in the art and can be used, including for instance IgV_{K}3-20/IgJ_{K}1, IgV_{K}3-15/IgJ_{K}1, and IgVλ3-21/IgJλ3.

IgV_{K}1-39 is short for Immunoglobulin Variable Kappa 1-39 Gene. The gene is also known as Immunoglobulin Kappa Variable 1-39; IGKV139; IGKV1-39. External Ids for the gene are HGNC: 5740; Entrez Gene: 28930; Ensembl: ENSG00000242371. A preferred amino acid sequence for IgV_{K}1-39 is given as SEQ ID NO: 107. This is the sequence of the V-region. The V-region can be combined with one of five J-regions. Two preferred joined sequences are indicated as IGKV1-39/jk1 and IGKV1-39/jk5; alternative names are IgV_{K}1-39*01/IGJ_{K}1*01 or IgV_{K}1-39*01/IGJ_{K}5*01 (nomenclature according to the IMGT database worldwide web at imgt.org). These names are exemplary and encompass allelic variants of the gene segments.

IgV_{K}3-20 is short for Immunoglobulin Variable Kappa 3-20 Gene. The gene is also known as Immunoglobulin Kappa Variable 3-20; IGKV320; IGKV3-20. External Ids for the gene are HGNC: 5817; Entrez Gene: 28912; Ensembl: ENSG00000239951. A preferred amino acid sequence for IgV_{K}3-20 is as SEQ ID NO: 108. This is the sequence of the V-region. The V-region can be combined with one of five J-regions. A preferred joined sequence is indicated as IGKV3-20/jk1; alternative name is IgV_{K}3-20*01/IGJ_{K}1*01 (nomenclature according to the IMGT database worldwide web at imgt.org). This name is exemplary and encompasses allelic variants of the gene segments.

IgV_{K}3-15 is short for Immunoglobulin Variable Kappa 3-15 Gene. The gene is also known as Immunoglobulin Kappa Variable 3-15; IGKV315; IGKV3-15. External Ids for the gene are HGNC: 5816; Entrez Gene: 28913; Ensembl: ENSG00000244437. A preferred amino acid sequence for IgV_{K}3-15 is given as SEQ ID NO: 109. This is the sequence of the V-region. The V-region can be combined with one of five J-regions. A preferred joined sequence is indicated as IGKV3-15/jk1; alternative name is IgV_{K}3-15*01/IGJ_{K}1*01 (nomenclature according to the IMGT database worldwide web at imgt.org). This name is exemplary and encompasses allelic variants of the gene segments.

IgVλ3-21 is short for Immunoglobulin Variable Lambda 3-21 Gene. The gene is also known as Immunoglobulin Lambda Variable 3-21; IGLV320; IGLV3-21. External Ids for the gene are HGNC: 5905; Entrez Gene: 28796; Ensembl: ENSG00000211662.2. A preferred amino acid sequence for IgVλ3-21 is given as SEQ ID NO: 110. This is the sequence of the V-region. The V-region can be combined with one of five J-regions. A preferred joined sequence is indicated as IGλV3-21/jk3; alternative name is IgVλ3-21/IGJ_{K}3 (nomenclature according to the IMGT database worldwide web at imgt.org). This name is exemplary and encompasses allelic variants of the gene segments.

The multivalent antibody is preferably a full length antibody comprising a constant region. Preferably, the multivalent antibody is a full length antibody comprising a constant region that is optimized for heterodimerization of heavy chains. Techniques for optimizing heterodimerization of heavy chains are known in the art and include, but are not limited to, the use of knob-in-hole mutations and the use of DEKK mutations (WO2013/157954 and De Nardis et al., J. Biol. Chem. (2017) 292(35) 14706-14717 incorporated herein by reference).

The multivalent antibody can be an antibody that induces effector function. The multivalent antibody can also be an antibody that does not induce effector function, or induces reduced effector function. The multivalent antibody preferably cannot induce effector function through Fc receptors. The second binding molecule preferably does not induce effector function or induces reduced effector function.

One type of effector function known in the art, is often referred to as antibody-dependent cellular cytotoxicity (ADCC), and also referred to as antibody-dependent cell-mediated cytotoxicity. ADCC is a mechanism of cell-mediated immune defense whereby an effector cell of the immune system actively lyses a target cell, whose membrane-surface antigens have been bound by specific antibodies. ADCC effector function is typically mediated by Fc receptors (FcRs). The receptors are key immune regulatory receptors connecting the antibody mediated (humoral) immune response to cellular effector functions. Receptors for all classes of immunoglobulins have been identified, including FcyR (IgG), FcεRI (IgE), FcaRI (IgA), FcµR (IgM) and FcδR (IgD). There are three classes of receptors for human IgG found on leukocytes: CD64 (FcyRI), CD32 (FcyRIIa, FcyRIIb and FcyRIIc) and CD16 (FcyRIIIa and FcyRIIIb). FcyRI is classed as a high affinity receptor (nanomolar range KD) while FcyRII and FcyRIII are low to intermediate affinity (micromolar range KD). In antibody dependent cellular cytotoxicity (ADCC), FcvRs on the surface of effector cells (natural killer cells, macrophages, monocytes and eosinophils) bind to the Fc region of an IgG which itself is bound to a target cell. Upon binding a signaling pathway is triggered which results in the secretion of various substances, such as lytic enzymes, perforin, granzymes and tumour necrosis factor, which mediate in the destruction of the target cell. The level of ADCC effector function varies for human IgG subtypes. Although this is dependent on the allotype and specific FcvR in simple terms ADCC effector function is high for human IgG1 and IgG3, and low for IgG2 and IgG4. Knowledge of the binding site of the FcvRs on the antibody has resulted in engineered antibodies that do not have ADCC effector functions.

Another type of effector function is independent on effector cells and is often referred to as complement-dependent cytotoxicity (CDC). This is an effector function of IgG and IgM antibodies. It is another mechanism of action by which therapeutic antibodies or antibody fragments can achieve an antitumor effect. CDC is initiated when C1q, the initiating component of the classical complement pathway, is fixed to the Fc portion of target-bound antibodies. This is the first step of a complex complement activation cascade that can ultimately result in the lysis of the antibody marked cell.

The second binding molecule preferably is a monospecific antibody comprising a constant region that is engineered to reduce ADCC and/or CDC activity of the antibody. Techniques to reduce ADCC and/or CDC activity of an antibody are known in the art and can suitably be employed in the present invention. In case the second binding molecule is an IgG1 antibody it is preferred that it comprises a modified CH2 region, the modification preferably being such that ADCC and/or CDC activity of the antibody is reduced or nullified. Some antibodies are modified in CH2/lower hinge region, for instance to reduce Fc-receptor interaction or to reduce C1q binding. The second binding molecule of the invention can be an IgG antibody with a mutant CH2 and/or lower hinge domain such that interaction of the second binding molecule to an Fc-receptor, preferably an Fc-gamma receptor is reduced.

The multivalent antibody can have a constant region that is engineered to reduce ADCC and/or CDC activity of the antibody. If the multivalent antibody is an IgG1 antibody it is preferred that it comprises a modified CH2 region, the modification preferably being such that ADCC and/or CDC activity of the antibody is reduced or nullified. Some antibodies are modified in CH2/lower hinge region, for instance to reduce Fc-receptor interaction or to reduce C1q binding. The multivalent antibody of the invention can be an IgG antibody with a mutant CH2 and/or lower hinge domain such that interaction of the multivalent antibody to an Fc-receptor, preferably an Fc-gamma receptor is reduced. A multivalent antibody exhibiting reduced effector function will through its binding with an immune cell engaging antigen remain capable of binding effector cells and activate these in the vicinity of an aberrant cell such as a cancer cell when bound via the TA1 and/or TA2 binding variable domain.

The invention thus provides a composition comprising a multivalent antibody and a second binding molecule as defined herein. The composition is preferably a therapeutic composition comprising the multivalent antibody and the second binding molecule, or a pharmaceutical composition comprising the multivalent antibody, the second binding molecule and a pharmaceutically acceptable carrier and/or diluent. The amount of multivalent antibody and second binding molecule in a composition according to the invention to be administered to a patient is typically in the therapeutic window, meaning that a sufficient quantity is used for obtaining a therapeutic effect, while the amount does not exceed a threshold value leading to an unacceptable extent of side-effects.

Also provided is a kit of parts comprising a multivalent antibody and a second binding molecule of the invention as defined herein. The kit of parts can comprise the multivalent antibody and second binding molecule of the invention as a single composition, or as separate compositions, i.e. one composition comprising the multivalent antibody and another composition comprising the second binding molecule. In certain embodiments, the kit comprises instructions for administering the multivalent antibody and second binding molecule simultaneously or consecutively to a subject in need thereof. In certain embodiments, the kit comprises instructions to administer the second binding molecule prior to administering the multivalent antibody.

The combination of a multivalent antibody and second binding molecule, the composition, or the kit of parts, as described herein, can be used for mitigating or reducing binding of the multivalent antibody to non-tumor cells, and/or for mitigating or reducing cell killing of non-tumor cells induced by the multivalent antibody.

In particular, in the context of the present invention, non-tumor cells express only one of the tumor-associated antigens that the multivalent antibody binds to, such as for example cells expressing TA1 but not TA2, and cells expressing TA2 but not TA1. Non-tumor cells expressing TA1 but not TA2, and non-tumor cells expressing TA2 but not TA1 can be present simultaneously. Reduced binding and reduced cell killing refer to a binding and a cell killing activity that is reduced when compared to the binding or cell killing activity of the multivalent antibody in the absence of the second binding molecule.

In certain embodiments, the present invention relates to a method for mitigating or reducing binding of a multivalent antibody as described herein to non-tumor cells, and/or for mitigating or reducing cell killing of non-tumor cells induced by the multivalent antibody as described herein, the method comprising using a second binding molecule as described herein that binds to TA1 or TA2 in conjunction with the multivalent antibody.

In certain embodiments, the present invention relates to the use of a second binding molecule as described herein for mitigating or reducing binding of a multivalent antibody as described herein to non-tumor cells, and/or for mitigating or reducing cell killing of non-tumor cells induced by the multivalent antibody as described herein.

In certain embodiments, the present invention relates to the use of a combination of a multivalent antibody and a second binding molecule as described herein for mitigating or reducing binding of the multivalent antibody to non-tumor cells, and/or for mitigating or reducing cell killing of non-tumor cells induced by the multivalent antibody.

In certain embodiments, the present invention relates to the use of a composition comprising a multivalent antibody and a second binding molecule as described herein for mitigating or reducing binding of the multivalent antibody to non-tumor cells, and/or for mitigating or reducing cell killing of non-tumor cells induced by the multivalent antibody. In certain embodiments, the composition is a therapeutic composition as described herein. In certain embodiments, the composition is a pharmaceutical composition as described herein.

In certain embodiments, the present invention relates to a combination of a multivalent antibody and a second binding molecule as described herein for use in mitigating or reducing binding of the multivalent antibody to non-tumor cells, and/or for mitigating or reducing cell killing of non-tumor cells induced by the multivalent antibody.

In certain embodiments, the present invention relates to a second binding molecule as described herein for use in mitigating or reducing binding of a multivalent antibody to non-tumor cells, and/or for mitigating or reducing cell killing of non-tumor cells induced by a multivalent antibody. In certain embodiments, the multivalent antibody is a multivalent antibody as described herein.

In certain embodiments, the present invention relates to a composition comprising a multivalent antibody and a second binding molecule as described herein for use in mitigating or reducing binding of the multivalent antibody to non-tumor cells, and/or for mitigating or reducing cell killing of non-tumor cells induced by the multivalent antibody. In certain embodiments, the composition is a therapeutic composition as described herein. In certain embodiments, the composition is a pharmaceutical composition as described herein.

In addition to their clinical use, a composition comprising a multivalent antibody and second binding molecule as described herein, a combination of a multivalent antibody and a second binding molecule as described herein, methods as described herein, and uses as described herein are useful in research and in development of therapeutic antibodies and compositions comprising such antibodies. This includes, but is not limited to, use in in vitro assays, in vivo experiments, including in vitro assays and in vivo experiments in preclinical characterization.

The combination of a multivalent antibody and second binding molecule, composition, or kit of parts, as described herein, can be used in a method for the treatment of a human or animal suffering from a medical indication, in particular cancer, which method comprises administering to a human or animal in need thereof a therapeutically effective amount of a combination of a multivalent antibody and second binding molecule of the invention as defined herein.

Provided is a method of treatment of cancer, wherein the method comprises:
administering to a subject in need thereof a multivalent antibody as described herein and additionally administering to the subject a second binding molecule as described herein;
administering to a subject in need thereof a composition as described herein;
administering to a subject in need thereof a therapeutic composition as described herein; or
administering to a subject in need thereof a pharmaceutical composition as described herein.

Further provided is a composition comprising a multivalent antibody and a second binding molecule as defined herein, or a kits of parts comprising a multivalent antibody and a second binding molecule as defined herein, for use in the treatment of cancer.

In certain embodiments, the present invention relates to the use of a combination of a multivalent antibody and a second binding molecule as described herein for use in the treatment of cancer.

In certain embodiments, the present invention relates to the use of a composition comprising a multivalent antibody and a second binding molecule as described herein for use in the treatment of cancer. In certain embodiments, the composition is a therapeutic composition as described herein. In certain embodiments, the composition is a pharmaceutical composition as described herein.

In certain embodiments, the present invention relates to a combination of a multivalent antibody and a second binding molecule as described herein for use in the treatment of cancer.

In certain embodiments, the present invention relates to a composition comprising a multivalent antibody and a second binding molecule as described herein for use in the treatment of cancer. In certain embodiments, the composition is a therapeutic composition as described herein. In certain embodiments, the composition is a pharmaceutical composition as described herein.

Further provided is the use of a combination of a multivalent antibody and second binding molecule of the invention as defined herein, composition comprising a multivalent antibody and a second binding molecule of the invention as defined herein, or a kits of parts comprising a multivalent antibody and a second binding molecule as defined herein, for the manufacture of a medicament for the treatment of an individual that has cancer. In certain embodiments, the composition is a therapeutic composition as described herein. In certain embodiments, the composition is a pharmaceutical composition as described herein.

Further provided is a method of treatment of cancer comprising administering to a subject in need thereof a multivalent antibody of the invention as defined herein, and additionally administering to the subject a second binding molecule of the invention as defined herein.

A multivalent antibody and second binding molecule of the invention can be administered simultaneously, as one composition or as separate compositions. A multivalent antibody and second binding molecule of the invention can also be administered sequentially, wherein the second binding molecule is administered first followed by the multivalent antibody, or vice versa. Preferably, the second binding molecule is administered prior to the multivalent antibody.

The cancer may be any solid or hematological cancer. Examples of solid cancers include those of epithelial origin; gynecological cancers such as ovarian and endometrial cancer; breast cancer; prostate cancer, and brain cancer.

Hematological cancer can be a leukemia or pre-leukemic disease, preferably of myeloid origin but also B cell lymphomas. Diseases that can be treated according to the invention include myeloid leukemias or pre-leukemic diseases such as acute myeloid leukemia (AML), myelodysplastic syndrome (MDS) and chronic myelogenous leukemia (CML), and Hodgkin’s lymphomas and most non-Hodgkin’s lymphomas. Also B-ALL; T-ALL, mantle cell lymphoma are also preferred targets for treatment with a composition or kit of parts of the invention.

Thus the invention provides a composition or a kit of parts in accordance with the appended claims for use as a pharmaceutical in the treatment of myelodysplastic syndrome (MDS), chronic myelogenous leukemia (CML), multiple myeloma (MM) or preferably acute myeloid leukemia (AML). Also provided is a use of a composition or a kit of parts of the claims in the preparation of a medicament for the treatment or prevention of MDS, CML, MM or preferably AML. It is preferred that the tumor antigen is CLEC12A.

The present invention further provides an expression vector comprising nucleic acid encoding the heavy chains and light chains of a multivalent antibody as described herein, as well as an expression vector comprising nucleic acid encoding the heavy chains and the light chains of a second binding molecule as described herein. The use of a single expression vector for both the multivalent antibody and second binding molecule is also envisaged.

The invention thus also relates to an expression vector comprising a nucleic acid encoding the heavy chain variable region of the first, second, and third variable domain of a multivalent antibody as defined herein, wherein the vector further comprises a nucleic acid encoding the heavy chain variable region of a second binding molecule as defined herein. The nucleic acid encoding the heavy chain variable region of a second binding molecule is a different nucleic acid than that encoding the heavy chain variable region of the first, second, and third variable domain of a multivalent antibody. For example, an exemplary multivalent antibody comprises a polypeptide comprising a binding domain that binds to an immune cell engaging antigen (IEA) and a binding domain that binds to TA1, and a polypeptide comprising a binding domain that binds to TA2. The nucleic acid encoding the heavy chain variable region of the second binding molecule does thus not encode a heavy chain variable region with specificity for TA2 but a heavy chain variable region with specificity for TA1.

Thus, in the embodiment wherein, in the multivalent antibody, the third variable domain that binds an immune cell engaging antigen (IEA) and the second variable domain that binds a second tumor antigen (TA2) are associated with an Fc region, and the first variable domain that binds a first tumor antigen (TA1) is linked to the third variable domain that binds an immune cell engaging antigen (IEA), the nucleic acid encoding the heavy chain variable region of the second binding molecule encodes a heavy chain variable region with specificity for TA1. In the embodiment wherein, in the multivalent antibody, wherein the third variable domain that binds an immune cell engaging antigen (IEA) and the first variable domain that binds a first tumor antigen (TA1) are associated with an Fc region, and the second variable domain that binds a second tumor antigen (TA2) is linked to the third variable domain that binds an immune cell engaging antigen (IEA), the nucleic acid encoding the heavy chain variable region of the second binding molecule encodes a heavy chain variable region with specificity for TA2.

The nucleic acid encoding the heavy chain variable region of the first, second, and third variable domain of a multivalent antibody as defined herein, and the nucleic acid encoding the heavy chain variable region of a second binding molecule as defined herein may further encode a heavy chain constant region, preferably comprising CH1, CH2 and CH3.

The expression vector may further comprise a nucleic acid encoding the light chain variable region of the first, second, and third variable domain of a multivalent antibody as defined herein, and a nucleic acid encoding the light chain variable region of a second binding molecule as defined herein. Such nucleic acids may further encode a light chain constant region (CL).

The invention further relates to a host cell comprising a nucleic acid encoding the heavy chain variable region of the first, second, and third variable domain of a multivalent antibody as defined herein, wherein the host cell further comprises a nucleic acid encoding the heavy chain variable region of a second binding molecule as defined herein. The nucleic acid encoding the heavy chain variable region of a second binding molecule is a different nucleic acid than that encoding the heavy chain variable region of the first, second, and third variable domain of a multivalent antibody, like explained above for the expression vector.

The nucleic acid encoding the heavy chain variable region of the first, second, and third variable domain of a multivalent antibody as defined herein, and the nucleic acid encoding the heavy chain variable region of a second binding molecule as defined herein may further encode a heavy chain constant region, preferably comprising CH1, CH2 and CH3.

The host cell may further comprise a nucleic acid encoding the light chain variable region of the first, second, and third variable domain of a multivalent antibody as defined herein, and a nucleic acid encoding the light chain variable region of a second binding molecule as defined herein. Such nucleic acids may further encode a light chain constant region (CL).

The host cell allows the expression of both the multivalent antibody and the second binding molecule from a single cell.

Examples of suitable vectors include plasmids, phagemids, cosmids, viruses and phage nucleic acids or other nucleic acid molecules that are capable of replication in a prokaryotic or eukaryotic host cell, e.g. a mammalian cell. The vector may be an expression vector wherein the nucleic acid encoding the heavy chains and light chains is operably linked to expression control elements. Typical expression vectors contain transcription and translation terminators, initiation sequences, and promoters useful for regulation of the expression of the polynucleotides.

It is preferred that the nucleic acid encoding the heavy chains of the multivalent antibody comprises one or more modifications that promote heterodimerization of the heavy chains. Such modifications are known in the art and include, but are not limited to, the examples of modifications as provided herein. The nucleic acid encoding the one or more heavy chains of the second binding molecule preferably does not have a modification that promotes heterodimerization. Instead it may comprise one or more modifications that promote homodimerization.

In the art various methods exist to produce antibodies and other types of binding molecules. Antibodies and binding molecules are typically produced by a cell that expresses nucleic acid encoding the antibody or binding molecule. The invention therefore also provides an isolated cell, or a cell in a tissue culture, that produces and/or comprises the antibody and/or second binding molecule of the invention. Typically this is an in vitro, isolated or recombinant cell. Such cell comprises a nucleic acid encoding the antibody and/or second binding molecule of the invention. The cell is preferably an animal cell, more preferably a mammalian cell, more preferably a primate cell, most preferably a human cell. For the purposes of the invention a suitable cell is any cell capable of comprising and preferably of producing an antibody according to the invention and/or comprising a nucleic acid according to the invention. Preferably, the cell is a hybridoma cell, a Chinese hamster ovary (CHO) cell, an NS0 cell or a PER.C6 cell. It is particularly preferred that the cell is a CHO cell.

Further provided is a cell culture, or cell line, comprising a cell according to the invention. Cell lines developed for industrial scale production of proteins and antibodies are herein further referred to as industrial cell lines.

The invention further provides a method for producing the multivalent antibody and/or second binding molecule of the invention, the method comprising culturing a cell of the invention and harvesting the multivalent antibody and/or second binding molecule from said culture. Said cell may be cultured in a serum free medium. Preferably said cell is adapted for suspension growth. The multivalent antibody and/or second binding molecule may be purified from the medium of the culture. Preferably said multivalent antibody and/or second binding molecule is affinity purified.

The present invention further provides a pharmaceutical composition comprising a multivalent antibody and second binding molecule as described herein, and a pharmaceutically acceptable carrier, diluent, or excipient.

When the multivalent antibody and/or second binding molecule of the present invention is formulated to be used as an injection or infusion solution for drip infusion, the injection or infusion solution may be in any form of an aqueous solution, suspension, or emulsion, or may be formulated as a solid agent together with pharmaceutically acceptable carrier such that that agent will be dissolved, suspended, or emulsified in a solvent at the time of use. Examples of the solvent that is used in the injection or the infusion solution for drip infusion include distilled water for injection, physiological saline, glucose solutions, and isotonic solutions (e.g., in which sodium chloride, potassium chloride, glycerin, mannitol, sorbitol, boric acid, borax, propylene glycol or the like is soluble).

Examples of pharmaceutically acceptable carriers include stabilizers, solubilizers, suspending agents, emulsifiers, soothing agents, buffering agents, preservatives, antiseptic agents, pH adjusters, and antioxidants. As stabilizers, various amino acids, albumin, globulin, gelatin, mannitol, glucose, dextran, ethylene glycol, propylene glycol, polyethylene glycol, ascorbic acid, sodium bisulfite, sodium thiosulfate, sodium edetate, sodium citrate, dibutylhydroxytoluene, or the like, can be used. As solubilizers, alcohols (e.g., ethanol), polyols (e.g., propylene glycol and polyethylene glycol), nonionic surfactants (e.g., Polysorbate 20 (registered trademark), Polysorbate 80 (registered trademark) and HCO-50), or the like, can be used. As suspending agents, glyceryl monostearate, aluminum monostearate, methyl cellulose, carboxymethyl cellulose, hydroxymethyl cellulose, sodium lauryl sulfate, or the like, can be used. As emulsifiers, gum arabic, sodium alginate, tragacanth, or the like, can be used. As soothing agents, benzyl alcohol, chlorobutanol, sorbitol, or the like, can be used. As buffering agents, phosphate buffer, acetate buffer, borate buffer, carbonate buffer, citrate buffer, Tris buffer, glutamic acid buffer, epsilon aminocaproic acid buffer, or the like, can be used. As preservatives, methyl parahydroxybenzoate, ethyl parahydroxybenzoate, propyl parahydroxybenzoate, butyl parahydroxybenzoate, chlorobutanol, benzyl alcohol, benzalkonium chloride, sodium dehydroacetate, sodium edeate, boric acid, borax, or the like, can be used. As antiseptic agents, benzalkonium chloride, parahydroxybenzoic acid, chlorobutanol, or the like, can be used. As the pH adjusters, hydrochloric acid, sodium hydroxide, phosphoric acid, acetic acid, or the like, can be used. As antioxidants, (1) aqueous antioxidants such as ascorbic acid, cysteine hydrochloride, sodium bisulfate, sodium metabisulfite, and sodium sulfite, (2) oil-soluble antioxidants such as ascorbyl palmitate, butylated hydroxy anisole, butylated hydroxy toluene, lecithin, propyl gallate, and α-tocopherol, or (3) metal chelating agents such as citric acid, ethylenediaminetetraacetic acid, sorbitol, tartaric acid, and phosphoric acid, can be used.

The injection or infusion solution for drip infusion can be produced by performing sterilization in the final process, or aseptic manipulation, e.g., sterilization by filtration with a filter and subsequently filling an aseptic container. The injection or infusion solution for drip infusion may be used by dissolving the vacuum dried or lyophilized aseptic powder (which may include a pharmaceutically acceptable carrier powder) in an appropriate solvent at the time of use.

The invention further provides a method of treating cancer in a subject comprising administering an effective amount of a multivalent antibody and second binding molecule, or a pharmaceutical composition, as described herein to a subject in need thereof. As such, the invention provides a combination of a multivalent antibody and second binding molecule as described herein for use in the treatment of cancer in a subject. The invention further provides a pharmaceutical agent for use in for preventing, suppressing symptom progression or recurrence of, and/or treating cancer, wherein the pharmaceutical agent comprises a multivalent antibody and a second binding molecule as described herein as an active ingredient.

A reference herein to a patent document or other matter which is given as background is not to be taken as an admission that that document or matter was known or that the information it contains was part of the common general knowledge at the priority date of any of the claims. The disclosure of each reference set forth herein is incorporated herein by reference in its entirety.

For the purpose of clarity and a concise description features are described herein as part of the same or separate embodiments, however, it will be appreciated that the scope of the invention may include embodiments having combinations of all or some of the features described.

### EXAMPLES

### Example 1

### Cells and Cell Lines

HCT116 (ECACC 91091005) is a human colon carcinoma cell line. BxPC3 (BxPC-3 ATCC ® CRL-1687) are human pancreatic cancer cells. BxPC3 cells express relatively high levels of EGFR and PD-L1, whereas HCT116 express lower levels of EGFR and PD-L1.

### Antibodies

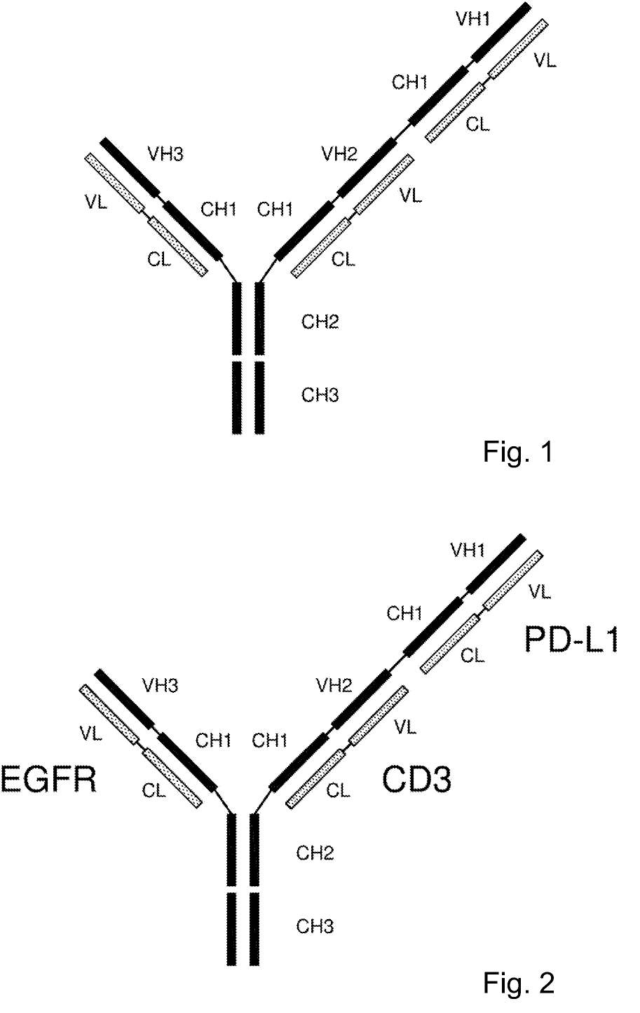

The multivalent antibodies that are prepared herein are trispecific antibodies that have two heavy chains with the general structure as depicted in FIG. 1.

Different trispecific antibodies were produced comprising different VH1 and VH2 regions, and the same VH3 region. The single VH3 region was selected from Fabs specific for EGFR (SEQ ID NO: 56); the two VH2 regions from Fabs specific for CD3 (SEQ ID Nos: 8 and 22), and the two VH1 regions from Fabs specific for PD-L1 (SEQ ID Nos: 38 and 42). The two selected PD-L1 Fabs have a relative affinity higher than the PD-L1 Fab used for one of the monospecific PD-L1 antibodies (comprising SEQ ID NO: 47) and lower than the monospecific PD-L1 antibody comprising heavy chains having SEQ ID NO: 46.

The heavy chains have a heterodimerization domain as described in WO2013/157954 and WO2013/157953. The heavy chain with VH3 has the CH3 domain with the DE residues 351D and 368E. The heavy chain with VH2 and VH1 has the complementary CH3 domain with the KK residues (351K, 366K) in the CH3 region according to EU numbering. Alternative inclusion of heterodimerization CH3 regions can be applied, either through use of different technology or having the KK residues on the VH3 side and the DE residues on the VH2 and VH1 side. Production of the two heavy chains in a cell leads to the generation of IgG heavy chain heterodimers of both heavy chains (WO2013/157954 and WO2013/157953).

The KK-heavy chain has the following N- to C-terminal structure VH1-CH1-linker-VH2-CH1-hinge-CH2-CH3. Expression vectors were made based on MV3032 (FIG. 10) to express the heavy and light chains in cells. The light chain used here was a common light chain comprising the IGKV1-39/jk1 variable region (sequence shown in FIG. 3).

The DE-heavy chain has the following N- to C-terminal structure VH3-CH1-hinge-CH2-CH3. Expression vectors to express the heavy and light chains in cells were made based on MV1625 (FIG. 11). The light chain encoded by this vector was a common light chain comprising the IGKV1-39/jk1 variable region (sequence shown in FIG. 3).

Three bivalent monospecific PD-L1 antibodies were produced: 1) an antibody comprising two heavy chains having an amino acid as set forth in SEQ ID NO: 46 and a light chain comprising an amino acid sequence as set forth in SEQ ID NO: 105; 2) an antibody comprising two heavy chains having an amino acid as set forth in SEQ ID NO: 47 and a light chain comprising an amino acid sequence as set forth in SEQ ID NO: 98; and 3) an antibody comprising two heavy chains having an amino acid as set forth in SEQ ID NO: 51 and a light chain comprising an amino acid sequence as set forth in SEQ ID NO: 106.

### Antibody Production

Hek293 cells were used for expression of the trispecific antibodies and monospecific PD-L1 antibody comprising heavy chains having SEQ ID NO: 47 and light chains having SEQ ID NO: 98. Two days before transfection, Hek293 cell stock was split in 293 culture medium in a 1:1 ratio and incubated overnight at 37° C. and 8% CO₂ at an orbital shaking speed of 155 rpm. Cells were diluted on the day before transfection to a density of 5 x 10e5 cells/mL. The suspension cells were seeded into plates, covered with a breathable seal and incubated overnight at 37° C. and 8% CO₂ at an orbital shaking speed of 285 rpm. On transfection day, 293-F culture medium was mixed with polyethylenimine (PEI) linear (MW 25,000). For each IgG to be produced, the 293F culture medium-PEI mix was added to the respective expression vector DNA (for IgG heterodimers DNA encoding each heavy chain). The mixture was incubated for 20 minutes at room temperature before gently adding to the cells. On the day after transfection, Penicillin-Streptomycin (Pen Strep) diluted in 293-F medium was added to each culture. The cultures were incubated at 37° C. and 8% CO₂ at an orbital shaking speed of 285 rpm until harvest seven days after transfection. Cultures were centrifuged 5 min at 500 g, supernatants containing IgGs were filtered using 10-12 µm melt blown polypropylene filter plates and stored at -20° C. prior to purification.

Supernatants were mixed with 1M Trizma pH8 and Protein A Sepharose CL-4B beads (50% v/v, G.E Healthcare Life Sciences) and incubated at 25° C. for 2 h at 600 rpm orbital shaking. Beads were vacuum filtered and washed 2 times with PBS pH7.4. The elution of the antibodies was performed by adding citrate buffer 0.1 M, pH3 followed by neutralization with 1M Trizma pH8. Purified IgG fractions were immediately buffer exchanged to PBS pH7.4. IgG samples were transferred into a 30 kDa filter , polyethersulfone membrane and centrifuged at 1500 g 4° C., PBS was added to the retentate, samples were mixed at 500 rpm for 3 min before IgGs were collected for storage at 4° C. IgG concentration was determined by Octet and Protein A biosensors (Pall ForteBio). Human IgG was used as standard in seven 2 folds dilutions. Concentrations of IgG samples were determined in duplicate.

Monospecific PD-L1 antibody comprising two heavy chains having an amino acid as set forth in SEQ ID NO: 46 and a light chain comprising an amino acid sequence as set forth in SEQ ID NO: 105, and monospecific PD-L1 antibody comprising two heavy chains having an amino acid as set forth in SEQ ID NO: 51 and a light chain comprising an amino acid sequence as set forth in SEQ ID NO: 106, were produced in CHO cells.

### Cytotoxicity Assay

The BxPC3 and HCT116 cell lines are used to measure T-cell mediated cell killing activity.

Using Ficoll and EasySep human T cell isolation kit according to standard techniques, resting T cells were isolated from whole blood from healthy donors, checked for > 95% T cell purity by anti-CD3 antibody using flow cytometric analysis and subsequently cryopreserved. The cryopreserved T cells were thawed and used if their viability was > 90% upon thawing, determined by standard Trypan Blue staining.

Cytotoxicity assay in short, thawed resting T cells and BxPC3 or HCT116 target cells were co-cultured in an E:T ratio of 5:1 for 48 hours. Target cell lysis was determined by measuring the fraction of alive cells by measuring ATP levels assessed by CellTiter-Glo (Promega). ATP levels, measured by luminescence on an Envision Microplate reader results in Relative light unit (RLU) values, which were analyzed using GraphPad Prism.

Target cell lysis for each sample was calculated as follows:

$$\text{\%Killing = (100} - \text{(RLUsample/RLU no IgG)} \times \text{100)} \text{.}$$

In a first experiment, the BxPC3 cytotoxicity assay was used to demonstrate the effect of the addition of a monospecific anti-PD-L1 antibody on the capability of the trispecific antibodies to induce killing of target cells. For the trispecific antibodies and control, a 20-fold 4-step dilution series was used, starting at a concentration of 20.5 nM.

Human T cells were co-cultured with BxPC3 target cells and incubated with two different PD-L1 = CD3 x EGFR trispecific antibodies. A first trispecific antibody comprised a PD-L1 binding domain comprising a heavy chain variable region having SEQ ID NO: 38; a CD3 binding domain comprising a heavy chain variable region having SEQ ID NO: 8; and an EGFR binding domain comprising a heavy chain variable region having SEQ ID NO: 56. A second trispecific antibody comprised a PD-L1 binding domain comprising a heavy chain variable region having SEQ ID NO: 42; a CD3 binding domain comprising a heavy chain variable region having SEQ ID NO: 22; and an EGFR binding domain comprising a heavy chain variable region having SEQ ID NO: 56. The percentage cell killing is set off against a negative control Tetanus Toxin (TT) = CD3 x TT trispecific antibody, the TT binding arm comprising a heavy chain variable region having SEQ ID NO 68: and the CD3 binding domain comprising a heavy chain variable region having SEQ ID NO: 8 or SEQ ID NO: 22. The cell killing activity of the first and second trispecific antibodies was compared with the cell killing activity of a trispecific PD-L1 = CD3 x mock antibody, wherein the mock arm had specificity against TT.

FIG. 6 shows that both the first trispecific antibody and the trispecific PD-L1=CD3 x mock antibody induce T cell-mediated cell killing at similar levels in the absence of a monospecific PD-L1 antibody (FIG. 6A; right column). The same applies to the second trispecific antibody (FIG. 6B; right column). Thus, both the trispecific antibody comprising a PD-L1 binding domain and an EGFR binding domain, and the trispecific antibody comprising a PD-L1 binding domain but lacking an EGFR binding domain, induce T cell-mediated cell killing in the absence of a monospecific PD-L1 antibody. This indicates that the T cell-mediated cell killing activity of the antibodies occurs independent from binding to EGFR, meaning that cells expressing PD-L1 but no or low levels of EGFR, (which would include non-tumor cells), will be killed by these antibodies, including the trispecific antibody comprising PD-L1 and EGFR binding domains.

Increasing the amount of monospecific PD-L1 antibody affects the activity of the trispecific PD-L1=CD3 x mock antibody but not of the trispecific antibodies comprising a PD-L1 and EGFR binding domain. The trispecific antibody comprising a PD-L1 binding domain and an EGFR binding domain still induces T cell-mediated cell killing in the presence of a monospecific PD-L1 antibody but the trispecific antibody comprising a PD-L1 binding domain but lacking an EGFR binding domain does not or less efficiently. This indicates that the cell killing activity of the antibodies is dependent on binding to EGFR in the presence of a monospecific PD-L1 antibody. This means that cells expressing PD-L1 but no or low amounts of EGFR, (e.g. non-tumor cells), will not or less efficiently be killed by the trispecific antibodies comprising a PD-L1 and EGFR binding domain when a monospecific PD-L1 antibody is present.

The results show that, in this assay, the bivalent monospecific PD-L1 antibody is capable of preventing T cell-mediated target cell killing by the trispecific antibody when EGFR is not or to a lesser extent bound by the trispecific antibody. In other words, the bivalent monospecific PD-L1 antibody is able to reduce T cell-mediated cell killing of cells that express PD-L1 but do not express EGFR or express only a low amount of EGFR. By combining the trispecific antibody with a bivalent monospecific antibody the trispecific antibody is more specifically targeted to the desired TA1, TA2 positive target cell.

In a second experiment, a cytotoxicity assay was used to determine the effect of a monospecific antibody on the capability of trispecific antibodies to induce T cell-mediated killing of HCT116 cells and BxPC3 cells. For the trispecific antibodies and control, an 8-fold 8-step dilution was used, starting at a concentration of 20.5 nM.

Human T cells were co-cultured with BxPC3 or HCT116 target cells in the presence of three different PD-L1 = CD3 x EGFR trispecific antibodies. A first trispecific antibody comprised a PD-L1 binding domain comprising a heavy chain variable region having SEQ ID NO: 38; a CD3 binding domain comprising a heavy chain variable region having SEQ ID NO: 8; and an EGFR binding domain comprising a heavy chain variable region having SEQ ID NO: 56. A second trispecific antibody comprised a PD-L1 binding domain comprising a heavy chain variable region having SEQ ID NO: 38[5359]; a CD3 binding domain comprising a heavy chain variable region having SEQ ID NO: 22; and an EGFR binding domain comprising a heavy chain variable region having SEQ ID NO: 56. A third trispecific antibody comprised a PD-L1 binding domain comprising a heavy chain variable region having SEQ ID NO: 42; a CD3 binding domain comprising a heavy chain variable region having SEQ ID NO: 22; and an EGFR binding domain comprising a heavy chain variable region having SEQ ID NO: 56. Apart from the PD-L1 = CD3 x TT negative control, also a TT = CD3 x EGFR control was included. The percentage cell killing is again set off against a negative control TT = CD3 x TT trispecific antibody, the TT binding arm comprising SEQ ID NO: 68, and the CD3 binding domain comprising SEQ ID NO: 8 or 22.

FIG. 7 shows that the trispecific PD-L1 = CD3 x mock antibody induces T cell-mediated cell killing in the absence of a monospecific PD-L1 antibody. This T cell-mediated target cell killing is due to the antibody binding to PD-L1 only, which can be on normal, non-tumor, cells expressing PD-L1 but no or low amounts of EGFR as well as on tumor cells expressing both PD-L1 and EGFR. This T cell -mediated target cell killing is strongly reduced or diminished in the presence of a monospecific PD-L1 antibody. This is believed to be due to less PD-L1 being available to the trispecific PD-L1 = CD3 x mock antibody as it has to compete with the monospecific PD-L1 antibody.

The PD-L1 = CD3 x EGFR trispecific antibody also induces T cell-mediated cell killing in the absence of a monospecific PD-L1 antibody. This T cell-mediated cell killing is not reduced, or to a lesser extent, compared to the trispecific PDL1 = CD3 x mock antibody, in the presence of a monospecific PD-L1 antibody. Also here, less PD-L1 will be available for binding of the trispecific PD-L1 = CD3 x EGFR antibody due to the monospecific PD-L1 antibody. However, since the PD-L1 = CD3 x EGFR trispecific antibody also binds EGFR the PD-L1 = CD3 x EGFR trispecific antibody can exhibit its cell killing effect on cells expressing both EGFR and PD-L1 but not or to a lesser extent on cells not expressing EGFR or expressing only low amounts of EGFR.

The combination of the trispecific antibodies with the bivalent monospecific anti-PD-L1 antibody comprising heavy chains having a sequence as set forth in SEQ ID NO: 46 was compared to the use of the trispecific antibodies only. The bivalent monospecific anti-PD-L1 antibody was added at a fixed 1:10 ratio of tri- to mono- specific antibody such that the monospecific antibody was always present in a significant surplus relative to the trispecific antibodies. TT = CD3 x EGFR control antibodies lacking a functional PD-L1 variable domain induce T cell-mediated target cell killing to some extent but are much less effective than trispecific antibodies with a functional PD-L1 variable domain (see FIG. 7; PD-L1 = CD3 x EGFR; or PD-L1 = CD3 x TT). The PD-L1 = CD3 x EGFR antibodies still induce T cell-mediated target cell killing in the presence of the bivalent monospecific PD-L1 antibody , even though they do not, or to a lesser extent, bind to EGFR negative PD-L1 positive cells. This is contrary to the PD-L1 = CD3 x mock antibodies that lose most of their T cell -mediated target cell killing activity in the presence of the bivalent monospecific antibody. The fact that they lose activity shows that the bivalent monospecific PD-L1 antibody adds significant specificity to the action of the trispecific PD-L1 = CD3 x EGFR antibodies. The monospecific PD-L1 antibody improves the therapeutic window of the trispecific antibodies.

On average HCT116 cells have lower EGFR and PD-L1 levels than BxPC3 cells. This does not change the effect of the monospecific PD-L1 antibody on the more specific cell targeting of the trispecific antibody albeit that the amount of T cell -mediated cell killing activity of the trispecific antibody is somewhat lower in the presence of the monospecific PD-L1 antibody (the lower panels of FIG. 7).

In a third experiment, the BxPC3 cytotoxicity assay was used to determine the effect of different ratios of trispecific antibody to monospecific antibody on their capability to kill BxPC3 cells. For the trispecific antibodies and control, a 3-fold 8-step dilution was used, starting at a concentration of 20.5 nM.

Human T cells were co-cultured with BxPC3 target cells in the presence of two different PD-L1 = CD3 x EGFR trispecific antibodies. A first trispecific antibody comprised a PD-L1 binding domain comprising a heavy chain variable region having SEQ ID NO: 38; a CD3 binding domain comprising a heavy chain variable region having SEQ ID NO: 8; and an EGFR binding domain comprising a heavy chain variable region having SEQ ID NO: 56. A second trispecific antibody comprised a PD-L1 binding domain comprising a heavy chain variable region having SEQ ID NO: 42[5426]; a CD3 binding domain comprising a heavy chain variable region having SEQ ID NO: 22; and an EGFR binding domain comprising a heavy chain variable region having SEQ ID NO: 56. Two different bivalent monospecific PD-L1 antibodies were tested: one comprising heavy chains having an amino acid sequence as set forth in SEQ ID NO: 46 (FIG. 8A) and one comprising heavy chains having an amino acid sequence as set forth in SEQ ID NO: 51 (FIG. 8B).

FIG. 8 shows that the presence of a bivalent monospecific antibody reduced T cell -mediated target cell killing of the trispecific antibody lacking an EGFR binding arm but not of the trispecific antibody comprising an EGFR binding arm. This indicates that the T cell mediated cell killing is higher when the trispecific antibody binds to PD-L1 and EGFR on cells expressing both PD-L1 and EGFR compared to when the trispecific antibody binds to PD-L1 only, in the presence of a bivalent monospecific PD-L1 antibody. From this it can be concluded that the monospecific PD-L1 antibody ensures that the T cell-mediated target cell killing is mainly, or to a higher extent, induced by the antibody binding to PD-L1 and EGFR positive cells and not by the antibody binding to PD-L1 positive and EGFR negative cells. The results were similar for the two different bivalent monospecific antibodies tested.

A fourth experiment is a repeat of the third experiment but then including another trispecific antibody comprising a PD-L1 binding domain comprising a heavy chain variable region having SEQ ID NO: 38; a CD3 binding domain comprising a heavy chain variable region having SEQ ID NO: 22; and an EGFR binding domain comprising a heavy chain variable region having SEQ ID NO: 56; and only using the bivalent monospecific antibodies comprising heavy chains having an amino acid sequence as set forth in SEQ ID NO: 46. For the trispecific antibodies and control, an 8-fold 8-step dilution was used, starting at a concentration of 20.5 nM.

FIG. 9 shows that this trispecific antibody leads to similar results as the other two trispecific antibodies. It can thus be concluded that trispecific antibodies having different PD-L1 and/or CD3 binding domains achieve the same result.

### Aspects of the Present Invention

1. A composition comprising a multivalent antibody comprising a first variable domain that binds a first tumor antigen (TA1), a second variable domain that binds a second tumor antigen (TA2) and a third variable domain that binds an immune cell engaging antigen (IEA); and wherein the composition further comprises a second binding molecule that binds TA1 or TA2.

2. The composition of aspect 1, wherein the third variable domain that binds an immune cell engaging antigen (IEA) and the second variable domain that binds a second tumor antigen (TA2) are associated with an Fc region, and the first variable domain that binds a first tumor antigen (TA1) is linked to the third variable domain that binds an immune cell engaging antigen (IEA).

3. The composition of aspect 1, wherein the third variable domain that binds an immune cell engaging antigen (IEA) and the first variable domain that binds a first tumor antigen (TA1) are associated with an Fc region, and the second variable domain that binds a second tumor antigen (TA2) is linked to the third variable domain that binds an immune cell engaging antigen (IEA).

4. The composition of any one of aspects 1-3, wherein the first, second, and/or third variable domain comprises a common light chain variable region.

5. The composition of any one of aspects 1-4, wherein the variable domain that binds an immune cell engaging antigen binds to CD3, TCR-α chain, TCR-β chain, CD2, CD4, CD5, CD7, CD8, CD137, CD28, CD16, CD16A, CD64, OX40, CD27, CD40, ICOS, GITR, NKG2D, NKp46, NKp44, or NKp30; preferably to CD3, TCR-α chain, TCR-β chain, CD2, or CD5; more preferably to CD3.

6. The composition of any one of aspects 1-5, wherein the variable domain that binds a first tumor associated antigen (TA1) binds to PD-L1, PD-L2, HVEM , CD47, B7-H3, B7-H4, B7-H7, or Siglec-15; preferably PD-L1 or PD-L2; more preferably PD-L1.

7. The composition of any one of aspects 1-6, wherein the variable domain that binds a second tumor associated antigen (TA2) binds to CLEC12A or EGFR, preferably EGFR.

8. The composition of any one of aspects 1-7, wherein the second binding molecule binds to TA1.

9. The composition of any one of aspects 1-8, wherein the second binding molecule is a bivalent monospecific antibody

10. The composition of aspect 9, wherein the second binding molecule has reduced effector function.

11. A kit of parts comprising a multivalent antibody and a second binding molecule as defined in any one of aspects 1-10.

12. A pharmaceutical composition comprising a multivalent antibody and a second binding molecule as defined in any one of aspects 1-10.

13. A combination of a multivalent antibody and a second binding molecule as defined in any one of aspects 1-10, a composition as defined in any one of aspects 1-10, a kit of parts as defined in aspect 11, or a pharmaceutical composition as defined in aspect 12, for use in the treatment of a subject in need thereof, in particular a subject that has cancer.

14. A method of treatment of cancer comprising:
administering to a subject in need thereof a multivalent antibody as defined in any one of aspects 1-10 and additionally administering to the subject a second binding molecule as defined in any one of aspects 1-10; or
administering to a subject in need thereof a composition as defined in any one of aspects 1-10; or
administering to a subject in need thereof a pharmaceutical composition as defined in aspect 12.

15. Use of a composition comprising a multivalent antibody and a second binding molecule as defined in any one of aspects 1-10, or a kits of parts comprising a multivalent antibody and a second binding molecule as defined in any one of aspects 1-10, for the manufacture of a medicament for the treatment of an individual that has cancer.

16. The combination for use in the treatment as defined in aspect 13, the method of treatment as defined in aspect 14, or the use as defined in aspect 15, wherein the multivalent antibody and second binding molecule are administered simultaneously as a single composition or as two separate compositions.

17. The combination for use in the treatment as defined in aspect 13, the method of treatment as defined in aspect 14, or the use as defined in aspect 15, wherein the multivalent antibody is administered prior to the second binding molecule.

18. The combination for use in the treatment as defined in aspect 13, the method of treatment as defined in aspect 14, or the use as defined in aspect 15, wherein the second binding molecule is administered prior to the multivalent antibody.

19. A vector comprising a nucleic acid encoding the heavy chain variable region of the first, second, and third variable domain of a multivalent antibody as defined in any one of aspects 1-10, wherein the vector further comprises a different nucleic acid encoding the heavy chain variable region of a second binding molecule as defined in any one of aspects 1-10.

20. Host cell comprising a nucleic acid encoding the heavy chain variable region of the first, second, and third variable domain of a multivalent antibody as defined in any one of aspects 1-10, wherein the host cell further comprises a different nucleic acid encoding the heavy chain variable region of a second binding molecule as defined in any one of aspects 1-10.

### Sequences

SEQ ID NO: 1: Heavy chain variable region

EVQLVQSGAEVKKPGSSVKVSCKASGGTFRSFGISWVRQAPGQGLEWMGG
FIPVLGTANYAQKFQGRVTIIADKSTNTAYMELSSLRSEDTAVYYCARRG
NWNPFDPWGQGTLVTVSS

SEQ ID NO: 2: HCDR1 according to Kabat

SFGIS

SEQ ID NO: 3: HCDR2 according to Kabat

GFIPVLGTANYAQKFQG

SEQ ID NO: 4: HCDR3 according to Kabat

RGNWNPFDP

SEQ ID NO: 5: Heavy chain variable region

QVQLVQSGAEVKKPGSSVKVSCKASGDAFKSKTFTISWVRQAPGQGLEWL
GGIIPLFGTITYAQKFQGRVTITADKSTNTAFMELSSLRSEDTAMYYCTR
RGNWNPFDPWGQGTLVTVSS

SEQ ID NO: 6: HCDR1 according to Kabat

SKTFTIS

SEQ ID NO: 7: HCDR2 according to Kabat

GIIPLFGTITYAQKFQG

SEQ ID NO: 8: Heavy chain variable region

EVQLVQSGSELKKPGSSVKVSCKASGVTFNSRTFTISWVRQAPGQGLEWL
GSIIPIFGTITYAQKFQGRVTITADKSTSTAFMELTSLRSEDTAIYYCTR
RGNWNPFDPWGQGTLVTVSS

SEQ ID NO: 9: HCDR1 according to Kabat

SRTFTIS

SEQ ID NO: 10: HCDR2 according to Kabat

SIIPIFGTITYAQKFQG

SEQ ID NO: 11: Heavy chain variable region

QVQLVQSGGGLVQPGGSLRLSCATSGFKFSSYALSWVRQAPGKGLEWVSG
ISGSGRTTWYADSVKGRFTISRDNSKNTLYLQMNSLRAEDTAVYYCARDG
GYSYGPYWYFDLWGRGTLVTVSS

SEQ ID NO: 12: HCDR1 according to Kabat

SYALS

SEQ ID NO: 13: HCDR2 according to Kabat

GISGSGRTTWYADSVKG

SEQ ID NO: 14: HCDR3 according to Kabat

DGGYSYGPYWYFDL

SEQ ID NO: 15: Heavy chain variable region

EVQLVQSGAEVKKPGESLKISCKGSGYSFTRFWIGWVRQMPGKGLEWMGI
IYPGDSDTRYSPSFQGQVTISADKSTSTAYLQWSSLKASDTGMYYCVRHI
RYFDWSEDYHYYLDVWGKGTTVTVSS

SEQ ID NO: 16: HCDR1 according to Kabat

RFWIG

SEQ ID NO: 17: HCDR2 according to Kabat

IIYPGDSDTRYSPSFQG

SEQ ID NO: 18: HCDR3 according to Kabat

HIRYFDWSEDYHYYLDV

SEQ ID NO: 19: Heavy chain variable region

EVQLVESGAEVKKPGESLKISCKGSGYSFTRYWIGWVRQMPGKGLEWMGI
IYPGDSDTRYSPSFQGQVTISADKSISTAYLQWSSLKASDTAMYYCVRNI
RYFVWSEDYHYYMDVWGKGTTVTVSS

SEQ ID NO: 20: HCDR1 according to Kabat

RYWIG

SEQ ID NO: 21: HCDR3 according to Kabat

NIRYFVWSEDYHYYMDV

SEQ ID NO: 22: Heavy chain variable region

EVQLVESGGGLVQPGRSLRLSCATSGFNFDDYTMHWVRQAPGKGLEWVSD
ISWSSGSIGYADSVKGRFTISRDNAKNSLWLQMNSLRTEDTALYFCAKDH
RGYGDYEGGGFDYWGQGTLVTVSS

SEQ ID NO: 23: HCDR1 according to Kabat

DYTMH

SEQ ID NO: 24: HCDR2 according to Kabat

DISWSSGSIGYADSVKG

SEQ ID NO: 25: HCDR3 according to Kabat

DHRGYGDYEGGGFDY

SEQ ID NO: 26: Heavy chain variable region

EVQLVQSGAEVKKPGSSVKVSCKASGGIFSTYAISWVRQAPGQGLEWMGG
IIPIFDTPNYAQKFQGRVTITADKSTSTAYMDLSSLRSEDTAVYYCAKNV
RGYSAYDLDYWGQGTLVTVSS

SEQ ID NO: 27: HCDR1 according to Kabat

TYAIS

SEQ ID NO: 28: HCDR2 according to Kabat

GIIPIFDTPNYAQKFQG

SEQ ID NO: 29: HCDR3 according to Kabat

NVRGYSAYDLDY

SEQ ID NO: 30: Heavy chain variable region

QVQLVQSGSELKKPGASVKVSCKASGYTFTSYSMNWVRQAPGQGLEWMGW
INNTGNPTYAQGFTGRFVFSLDTSVSTAYLQISSLKAEDTAVYYCARDHD
FRTGRFDIWGQGTTVTVSS

SEQ ID NO: 31: HCDR1 according to Kabat

SYSMN

SEQ ID NO: 32: HCDR2 according to Kabat

WINTNTGNPTYAQGFTG

SEQ ID NO: 33: HCDR3 according to Kabat

DHDFRTGRAFDI

SEQ ID NO: 34: Heavy chain variable region

EVQLVESGGDVVQPGRSLRLSCAASGFTFSSYGMHWVRQAPGKGLEWVAV
ISYGSNKYYADSVKGRFTISRDNSKSTLFLQMNSLRAEDTAVYFCVRGLP
ITMVRGYSFDYWGQGTLVTVSS

SEQ ID NO: 35: HCDR1 according to Kabat

SYGMH

SEQ ID NO: 36: HCDR2 according to Kabat

VISYDGSNKYYADSVKG

SEQ ID NO: 37: HCDR3 according to Kabat

GLPITMVRGAYSFDY

SEQ ID NO: 38: Heavy chain variable region

EVQLVQSGAEVKKPGSSVKVSCKASGDTFNTYSITWVRQAPGQGLEWMGS
IVPIGTINNAQKFQGRVTITADKSANTAYMELSSLRSEDTAVYYCARDNT
MVRGVDYYMDVWGKGTMVTVSS

SEQ ID NO: 39: HCDR1 according to Kabat

TYSIT

SEQ ID NO: 40: HCDR2 according to Kabat

SIVPIFGTINNAQKFQG

SEQ ID NO: 41: HCDR3 according to Kabat

DNTMVRGVDYYYMDV

SEQ ID NO: 42: Heavy chain variable region

QVQLVQSGAEVKKPGSSVKVSCKASGDTFRSYGITWVRQAPGQGLEWMGG
IIPIFGTTNYAQKFQGRVTITADKSTSTVYMELSSLRSEDTAVYYCARRR
GYSNPHWLDPWGQGTLVTVSS

SEQ ID NO: 43: HCDR1 according to Kabat

SYGIT

SEQ ID NO: 44: HCDR2 according to Kabat

GIIPIFGTTNYAQKFQG

SEQ ID NO: 45: HCDR3 according to Kabat

RRGYSNPHWLDP

SEQ ID NO: 46: Heavy chain sequence

EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSAASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTCPPCPAPELGRGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGN

SEQ ID NO: 47: Heavy chain

QVQLVQSGAEVKKPGSSVRVSCKASGGTFNTYAINWVRQAPGQGLEWVGR
IIPIFGTANYAQKFQGRVTISADKSTTTAYMELSSLRSEDTAVFYCAKDE
TGYSSSNFQHWGRGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKRVEPKSCDKTHTCPPCPAPELGRGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPP
VLDSDGSFFLYSKLTVDKSRWQQGN

SEQ ID NO: 48: HCDR1 according to Kabat

TYAIN

SEQ ID NO: 49: HCDR2 according to Kabat

RIIPIFGTANYAQKFQG

SEQ ID NO: 50: HCDR3 according to Kabat

DETGYSSSNFQH

SEQ ID NO: 51: Heavy chain sequence

EVQLVESGGGLVQPGGSLRLSCAASGFTFSRYWMSWVRQAPGKGLEWVAN
IKQDGSEKYYVDSVKGRFTISRDNAKNSLYLQMNSLRAEDTAVYYCAREG
GWFGELAFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKRVEPKSCDKTHTCPPCPAPEFEGGPSVFLFPPK
PKDTLMISRTPEVTCWVDVSHEDPEVKFNWYVDGVEVHNAKTKPREEQYN
STYRVVSVLTVLHQDWLNGKEYKCKVSNKALPASIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK

SEQ ID NO: 52: Heavy chain variable region

QVQLVQSGAEVKKPGASVKVSCKASGYTFTSYGISWVRQAPGQGLEWMGW
ISAYNANTNYAQKLQGRVTMTTDTSTSTAYMELRSLRSDDTAVYYCAKDR
HWHWWLDAFDYWGQGTLVTVSS

SEQ ID NO: 53: HCDR1 according to Kabat

SYGIS

SEQ ID NO: 54: HCDR2 according to Kabat

WISAYNANTNYAQKLQG

SEQ ID NO: 55: HCDR3 according to Kabat

DRHWHWWLDAFDY

SEQ ID NO: 56: Heavy chain variable region

QVQLVQSGAEVKKPGASVKVSCKASGYTFTSYGISWVRQAPGQGLEWMGW
ISAYNANTNYAQKLQGRVTMTTDTSTSTAYMELRSLRSDDTAVYYCAKDL
YGHWWLDAFDYWGQGTLVTVSS

SEQ ID NO: 57: HCDR3 according to Kabat

DLYGHWWLDAFDY

SEQ ID NO: 58: Heavy chain variable region

QVQLVQSGAEVKKPGASVKVSCKASGYTFTSYGISWVRQAPGQGLEWMGW
ISAYNANTNYAQKLQGRVTMTTDTSTSTAYMELRSLRSDDTAVYYCAKGP
GSHWWLDAFDYWGQGTLVTVSS

SEQ ID NO: 59: HCDR3 according to Kabat

GPGSHWWLDAFDY

SEQ ID NO: 60: Heavy chain variable region

QVQLVQSGAEVKKPGASVKVSCKASGYTFTSYGISWVRQAPGQGLEWMGW
ISAYNANTNYAQKLQGRVTMTTDTSTSTAYMELRSLRSDDTAVYYCAKDR
GWHWWLDAFDYWGQGTLVTVSS

SEQ ID NO: 61: HCDR3 according to Kabat

DRGWHWWLDAFDY

SEQ ID NO: 62: Heavy chain variable region

QVQLVQSGAEVKKPGASVKVSCKASGYTFTSYGISWVRQAPGQGLEWMGW
ISAYNANTNYAQKLQGRVTMTTDTSTSTAYMELRSLRSDDTAVYYCAKDR
HWHWWLDGFDYWGQGTLVTVSS

SEQ ID NO: 63: HCDR3 according to Kabat

DRHWHWWLDGFDY

SEQ ID NO: 64: Heavy chain variable region

QVQLVQSGAEVKKPGASVKVSCKASGYTFTSYYMHWVRQAPGQGLEWMGI
INPSGGSTSYAQKFQGRVTMTRDTSTSTVYMELSSLRSEDTAVYYCAKGT
TGDWFDYWGQGTLVTVSS

SEQ ID NO: 65: HCDR1 according to Kabat

SYYMH

SEQ ID NO: 66: HCDR2 according to Kabat

IINPSGGSTSYAQKFQG

SEQ ID NO: 67: HCDR3 according to Kabat

GTTGDWFDY

SEQ ID NO: 68: Heavy chain variable region

EVQLVETGAEVKKPGASVKVSCKASDYIFTKYDINWVRQAPGQGLEWMGW
MSANTGNTGYAQKFQGRVTMTRDTSINTAYMELSSLTSGDTAVYFCARSS
LFKTET\PYYHFALDVWGQGTTVTVSS

SEQ ID NO: 69: Linker 1

ESKYGPP

SEQ ID NO: 70: Linker 2

EPKSCDKTHT

SEQ ID NO: 71: Linker 3

GGGGSGGGGS

SEQ ID NO: 72: Linker 4

ERKSSVESPPSP

SEQ ID NO: 73: Linker 5

ERKCSVESPPSP

SEQ ID NO: 74: Linker 6

ELKTPLGDTTHT

SEQ ID NO: 75: Linker 7

ESKYGPPSPSSP

SEQ ID NO: 76: Linker 8

ERKSSVEAPPVAG

SEQ ID NO: 77: Linker 9

ERKCSVEAPPVAG

SEQ ID NO: 78: Linker 10

ESKYGPPAPEFLGG

SEQ ID NO: 79: Linker 11

EPKSCDKTHTSPPSP

SEQ ID NO: 80: Linker 12

EPKSCDGGGGSGGGGS

SEQ ID NO: 81: Linker 13

GGGGSGGGGSAPPVAG

SEQ ID NO: 82: Linker 14

EPKSCDKTHTAPELLGG

SEQ ID NO: 83: Linker 15

ERKSSVESPPSPAPPVAG

SEQ ID NO: 84: Linker 16

ERKCSVESPPSPAPPVAG

SEQ ID NO: 85: Linker 17

ELKTPLGDTTHTAPEFLGG

SEQ ID NO: 86: Linker 18

ESKYGPPSPSSPAPEFLGG

SEQ ID NO: 87: Linker 19

EPKSCDKTHTSPPSPAPELLGG

SEQ ID NO: 88: Linker 20

ERKSSVEEAAAKEAAAKAPPVAG

SEQ ID NO: 89: Linker 21

ERKCSVEEAAAKEAAAKAPPVAG

SEQ ID NO: 90: Linker 22

ESKYGPPEAAAKEAAAKAPEFLGG

SEQ ID NO: 91: Linker 23

EPKSCDKTHTEAAAKEAAAKAPELLGG

SEQ ID NO: 92: Linker 24

ELKTPLGDTTHTEAAAKEAAAKAPEFLGG

SEQ ID NO: 93: Light chain variable region

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPTFGQ
GTKVEIK

SEQ ID NO: 94: LCDR1 according to IMGT

QSISSY

SEQ ID NO: 95: LCDR2 according to IMGT

AAS

SEQ ID NO: 96: LCDR3 according to IMGT

QQSYSTPPT

SEQ ID NO: 97: Light chain constant region

RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

SEQ ID NO: 98: Light chain sequence

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPTFGQ
GTKVEIKRTVAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

SEQ ID NO: 99: IGKV1-39/jk5 Light chain variable region

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPPITFG
QGTRLEIK

SEQ ID NO: 100: CH1 sequence

ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRV

SEQ ID NO: 101: Hinge

EPKSCDKTHTCPPCP

SEQ ID NO: 102: CH2 sequence

APELLGGPSVFLFPPKPKDTLMISRTPEVTCVVVDVSHEDPEVKFNWYVD
GVEHNAKTKPREEQYNSTYRVVSVLTVLHQDWLNGKEYKCKVSNKALPAP
IEKTIKAK

SEQ ID NO: 103: Modified CH3 sequence

GQPREPQVYTKPPSREEMTKNQVSLKCLVKGFYPSDIAVEWESNGQPENN
YKTTPPVLDSDGSFFLYSKLTVDKSRWQQGNVFSCSVMHEALHNHYTQKS
LSLSPGK

SEQ ID NO: 104: Modified CH3 sequence

GQPREPQVYTDPPSREEMTKNQVSLTCEVKGFYPSDIAVEWESNGQPENN
YKTTPPVLDSDGSFFLYSKLTVDKSRWQQGNVFSCSVMHEALHNHYTQKS
LSLSPGK

SEQ ID NO: 105: Light chain sequence

DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

SEQ ID NO: 106: Light chain sequence

EIVLTQSPGTLSLSPGERATLSCRASQRVSSSYLAWYQQKPGQAPRLLIY
DASSRATGIPDRFSGSGSGTDFTLTISRLEPEDFAVYYCQQYGSLPWTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC

SEQ ID NO: 107: IgVk1-39 V-region

DIQMTQSPSSLSASVGDRVTITCRASQSISSYLNWYQQKPGKAPKLLIYA
ASSLQSGVPSRFSGSGSGTDFTLTISSLQPEDFATYYCQQSYSTP

SEQ ID NO: 108: IgVk 3-20 V-region

EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSP

SEQ ID NO: 109: IgVk3-15 V-region

EIVMTQSPATLSVSPGERATLSCRASQSVSSNLAWYQQKPGQAPRLLIYG
ASTRATGIPARFSGSGSGTEFTLTISSLQSEDFAVYYCQQYNNWP

SEQ ID NO: 110: IgVL3-21 V-region

SYVLTQPPSVSVAPGETARITCGGDNIGRKSVYWYQQKSGQAPVLVIYYD
SDRPSGIPERFSGSNSGNTATLTISRVEAGDEADYYCQVWDGSSDH

1. A therapeutic composition comprising a multivalent antibody comprising a first variable domain that binds a first tumor antigen (TA1), a second variable domain that binds a second tumor antigen (TA2) and a third variable domain that binds an immune cell engaging antigen (IEA); and wherein the composition further comprises a second binding molecule that binds TA1 or TA2.

2. The therapeutic composition of claim 1, wherein the multivalent antibody comprises an Fc region.

3. The therapeutic composition of claim 1 or 2, wherein the third variable domain that binds an immune cell engaging antigen (IEA) and the second variable domain that binds a second tumor antigen (TA2) are associated with an Fc region, and the first variable domain that binds a first tumor antigen (TA1) is linked to the third variable domain that binds an immune cell engaging antigen (IEA).

4. The therapeutic composition of claim 1 or 2, wherein the third variable domain that binds an immune cell engaging antigen (IEA) and the first variable domain that binds a first tumor antigen (TA1) are associated with an Fc region, and the second variable domain that binds a second tumor antigen (TA2) is linked to the third variable domain that binds an immune cell engaging antigen (IEA).

5. The therapeutic composition of any one of claims 1-4, wherein the first, second, and/or third variable domain comprises a common light chain variable region.

6. The therapeutic composition of any one of claims 1-5, wherein the variable domain that binds an immune cell engaging antigen binds to CD3, TCR-a chain, TCR-β chain, CD2, CD4, CD5, CD7, CD8, CD137, CD28, CD16, CD16A, CD64, OX40, CD27, CD40, ICOS, GITR, NKG2D, NKp46, NKp44, or NKp30; preferably to CD3, TCR-a chain, TCR-β chain, CD2, or CD5; more preferably to CD3.

7. The therapeutic composition of any one of claims 1-6, wherein the variable domain that binds a first tumor associated antigen (TA1) binds to PD-L1, PD-L2, HVEM , CD47, B7-H3, B7-H4, B7-H7, or Siglec-15; preferably PD-L1 or PD-L2; more preferably PD-L1.

8. The therapeutic composition of any one of claims 1-7, wherein the variable domain that binds a second tumor associated antigen (TA2) binds to CLEC12A or EGFR, preferably EGFR.

9. The therapeutic composition of any one of claims 1-8, wherein the first tumor associated antigen (TA1) is expressed on non-tumor cells.

10. The therapeutic composition of any one of claims 1-9, wherein the second binding molecule binds to TA1.

11. The therapeutic composition of any one of claims 1-10, wherein the second binding molecule is a bivalent monospecific antibody.

12. The therapeutic composition of any one of claims 1-11, wherein the second binding molecule has a binding affinity to TA1 or TA2 that is comparable to, equal to, or lower than the binding affinity to TA1 or TA2 of the first variable domain or second variable domain of the multivalent antibody.

13. The therapeutic composition of any one of claims 1-12, wherein the second binding molecule has reduced effector function.

14. A kit of parts comprising a multivalent antibody and a second binding molecule as defined in any one of claims 1-13.

15. A kit of parts comprising a therapeutic composition of any one of claims 1-13 and instructions for administering the composition to a subject in need thereof.

16. The kit of parts of claim 14 or 15, wherein the kit comprises instructions for administering the multivalent antibody and second binding molecule simultaneously or consecutively to a subject in need thereof.

17. The kit of parts of any one of claims 14-16, wherein the kit comprises instructions to administer the second binding molecule prior to administering the multivalent antibody.

18. A pharmaceutical composition comprising a multivalent antibody and a second binding molecule as defined in any one of claims 1-13, and a pharmaceutically acceptable carrier, diluent, or excipient.

19. A combination of a multivalent antibody and a second binding molecule as defined in any one of claims 1-13, a composition comprising a multivalent antibody and a second binding molecule as defined in any one of claims 1-13, a therapeutic composition of any one of claims 1-13, a kit of parts of any one of claims 14-17, or a pharmaceutical composition of claim 18, for mitigating or reducing binding of the multivalent antibody to non-tumor cells, and/or for mitigating or reducing cell killing of non-tumor cells induced by the multivalent antibody.

20. A combination of a multivalent antibody and a second binding molecule as defined in any one of claims 1-13, a composition comprising a multivalent antibody and a second binding molecule as defined in any one of claims 1-13, a therapeutic composition of any one of claims 1-13, a kit of parts of any one of claims 14-17, or a pharmaceutical composition of claim 18, for use as a medicament.

21. A combination of a multivalent antibody and a second binding molecule as defined in any one of claims 1-13, a composition comprising a multivalent antibody and a second binding molecule as defined in any one of claims 1-13, a therapeutic composition of any one of claims 1-13, a kit of parts of any one of claims 14-17, or a pharmaceutical composition of claim 18, for use in the treatment of a subject in need thereof, in particular a subject that has cancer.

22. Use of a combination of a multivalent antibody and a second binding molecule as defined in any one of claims 1-13, a composition comprising a multivalent antibody and a second binding molecule as defined in any one of claims 1-13, a therapeutic composition of any one of claims 1-13, a kit of parts of any one of claims 14-17, or a pharmaceutical composition of claim 18, in the manufacture of a medicament for the treatment of cancer.

23. A method for mitigating or reducing binding of a multivalent antibody as defined in any one of claims 1-13 to non-tumor cells expressing TA1 or TA2, wherein the method comprises using a second binding molecule as defined in any one of claims 1-13 that binds to TA1 or TA2 in conjunction with the multivalent antibody.

24. The method of claim 23, wherein the non-tumor cells express TA1 and the second binding molecule binds to TA1.

25. The method of claim 23 or 24, wherein binding of the multivalent antibody to non-tumor cells expressing TA1 or TA2 is reduced as compared to binding of the multivalent antibody to non-tumor cells expressing TA1 or TA2 in a method not using the second binding molecule.

26. A method of treatment of cancer, wherein the method comprises:
administering to a subject in need thereof a multivalent antibody as defined in any one of claims 1-13 and additionally administering to the subject a second binding molecule as defined in any one of claims 1-13;
administering to a subject in need thereof a composition comprising a multivalent antibody and second binding molecule as defined in any one of claims 1-13; or
administering to a subject in need thereof a therapeutic composition of any one of claims 1-13; or
administering to a subject in need thereof a pharmaceutical composition of claim 18.

27. The combination, composition, therapeutic composition, or kit of parts for use of any one of claims 19-21, or the method of any one of claims 23-26, wherein the multivalent antibody and second binding molecule are administered simultaneously as a single composition or as two separate compositions.

28. The combination, composition, therapeutic composition, or kit of parts for use of any one of claims 19-21 or the method of any one of claims 23-26, wherein the multivalent antibody is administered prior to the second binding molecule.

29. The combination, composition, therapeutic composition, or kit of parts for use of any one of claims 19-21, or the method of any one of claims 23-26, wherein the second binding molecule is administered prior to the multivalent antibody.

30. A vector comprising a nucleic acid encoding the heavy chain variable region of the first, second, and third variable domain of a multivalent antibody as defined in any one of claims 1-13, wherein the vector further comprises a different nucleic acid encoding the heavy chain variable region of a second binding molecule as defined in any one of claims 1-13.

31. A host cell comprising a nucleic acid encoding the heavy chain variable region of the first, second, and third variable domain of a multivalent antibody as defined in any one of claims 1-13, wherein the host cell further comprises a different nucleic acid encoding the heavy chain variable region of a second binding molecule as defined in any one of claims 1-13.

32. The host cell of claim 31, wherein the host cell further comprises a nucleic acid encoding the light chain variable region of the first, second, and third variable domain of a multivalent antibody, and the light chain variable region of a second binding molecule, as defined in any one of claims 1-13.
